(19) United States
(12) Patent Application Publication
(10) Pub. No.: US 2019/0375764 A1
(43) Pub. Date: Dec. 12, 2019

(54) SALTS OF A HETEROCYCLIC COMPOUND AND CRYSTALLINE FORMS, PROCESSES FOR PREPARING, THERAPEUTIC USES, AND PHARMACEUTICAL COMPOSITIONS THEREOF

(71) Applicant: Sunovion Pharmaceuticals Inc., Marlborough, MA (US)

(72) Inventors: Nandkumar Nivritti BHOGLE, Shrewsbury, MA (US); Yuji FUJIWARA, Draper, UT (US); Kenjiro HIRA, Hyogo (JP); Tomoya KARASAWA, Osaka (JP); Kostas SARANTEAS, Littleton, MA (US); John R. SNOONIAN, Bolton, MA (US); Harold Scott WILKINSON, Westboro, MA (US); Haitao ZHANG, Shrewsbury, MA (US)

(21) Appl. No.: 16/433,361

(22) Filed: Jun. 6, 2019

Related U.S. Application Data
(60) Provisional application No. 62/681,960, filed on Jun. 7, 2018.

(51) Int. Cl. C07D 498/04; C07D 231/56

(52) U.S. Cl. CPC C07D 498/04; C07B 2200/13; C07D 231/56

(57) ABSTRACT

The present disclosure relates to salts of (R)-1-(3,4-dihydro-1H-[1,4]oxazino[4,3-b]indazol-1-yl)-N-methylmethanamine, crystalline forms thereof, and methods of preparation thereof, which are useful in the treatment of CNS disorders.

### CROSS REFERENCE TO RELATED APPLICATIONS

This application claims priority from U.S. provisional application 62/681,960, filed Jun. 7, 2018, the entire disclosure of which is hereby incorporated herein by reference.

### FIELD

This application relates to salts of (R)-1-(3,4-dihydro-1H-[1,4]oxazino[4,3-b]indazol-1-yl)-N-methylmethanamine (Compound 1), and crystalline forms, processes for preparing, therapeutic uses, and pharmaceutical compositions thereof.

### BACKGROUND

Central nervous system disorders affect a wide range of the population with differing severity. Neurological and psychiatric disorders include major depression, schizophrenia, bipolar disorder, obsessive compulsive disorder (OCD), panic disorder, and posttraumatic stress disorder (PTSD), among others. These disorders affect a person's thoughts, mood, behavior and social interactions and can significantly impair daily functioning. See, e.g., Diagnostic and Statistical Manual of Mental Disorders, 4th Ed., American Psychiatric Association (2000) (“DSM-IV-TR”); Diagnostic and Statistical Manual of Mental Disorders, 5th Ed., American Psychiatric Association (2013) (“DSM-5”).

Bipolar disorder is a serious psychiatric disorder that has a prevalence of approximately 2% of the population, and affects both genders alike. It is a relapsing-remitting condition characterized by cycling between elevated (i.e., manic) and depressed moods, which distinguishes it from other disorders such as major depressive disorder and schizophrenia. Bipolar I is defined by the occurrence of a full manic episode, although most individuals experience significant depression. Symptoms of mania include elevated or irritable mood, hyperactivity, grandiosity, decreased need for sleep, racing thoughts and in some cases, psychosis. The depressive episodes are characterized by anhedonia, sad mood, hopelessness, poor self-esteem, diminished concentration and lethargy. Bipolar II is defined as the occurrence of a major depressive episode and hypomanic (less severe mania) episode although patients spend considerably more time in the depressive state. Other related conditions include cyclothymic disorder.

Schizophrenia is a psychopathic disorder of unknown origin, which usually appears for the first time in early adulthood and is marked by characteristics such as psychotic symptoms, phasic progression and development, and/or deterioration in social behavior and professional capability. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotor disorders (e.g., catatonia). Other symptoms are also associated with this disorder.

Schizophrenia is classified into subgroups: the paranoid type, characterized by delusions and hallucinations and absence of thought disorder, disorganized behavior, and affective flattening; the disorganized type, also named “hebephrenic schizophrenia,” in which thought disorder and flat affect are present together; the catatonic type, in which prominent psychomotor disturbances are evident, and symptoms may include catatonic stupor and waxy flexibility; and the undifferentiated type, in which psychotic symptoms are present but the criteria for paranoid, disorganized, or catatonic types have not been met. The symptoms of schizophrenia normally manifest themselves in three broad categories: positive, negative and cognitive symptoms. Positive symptoms are those which represent an “excess” of normal experiences, such as hallucinations and delusions. Negative symptoms are those where the patient suffers from a lack of normal experiences, such as anhedonia and lack of social interaction. The cognitive symptoms relate to cognitive impairment in schizophrenics, such as lack of sustained attention and deficits in decision making.

Neurological and psychiatric disorders can exhibit a variety of symptoms, including cognitive impairment, depressive disorders, and anxiety disorders. Cognitive impairment includes a decline in cognitive functions or cognitive domains, e.g., working memory, attention and vigilance, verbal learning and memory, visual learning and memory, reasoning and problem solving (e.g., executive function, speed of processing and/or social cognition). In particular, cognitive impairment may indicate deficits in attention, disorganized thinking, slow thinking, difficulty in understanding, poor concentration, impairment of problem solving, poor memory, difficulties in expressing thoughts, and/or difficulties in integrating thoughts, feelings and behavior, or difficulties in extinction of irrelevant thoughts.

Depressive disorders include major depressive disorder and dysthymia, and are associated with depressed mood (sadness), poor concentration, insomnia, fatigue, appetite disturbances, excessive guilt and thoughts of suicide.

Anxiety disorders are disorders characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs.

Various drugs are currently being developed for the treatment of CNS disorders For example, the compound (R)-1-(3,4-dihydro-1H-[1,4]oxazino[4,3-b]indazol-1-yl)-N-methylmethanamine, which is reported in PCT Publication No: WO2016/130790, the entirety of which is incorporated herein by reference, is useful in the treatment of CNS disorders. There is a need for salts and new forms of (R)-1-(3,4-dihydro-1H-[1,4]oxazino[4,3-b]indazol-1-yl)-N-methylmethanamine for preparing pharmaceutically useful formulations and dosage forms with suitable properties related to, for example, facilitating the manufacture of safe, effective, and high quality drug products.

### SUMMARY

Provided herein are salts of (R)-1-(3,4-dihydro-1H-[1,4]oxazino[4,3-b]indazol-1-yl)-N-methylmethanamine and crystalline forms, processes for preparing, therapeutic uses, and pharmaceutical compositions.

In some embodiments, provided are salts, and crystalline forms thereof, of (R)-1-(3,4-dihydro-1H-[1,4]oxazino[4,3-b]indazol-1-yl)-N-methylmethanamine (Compound 1):

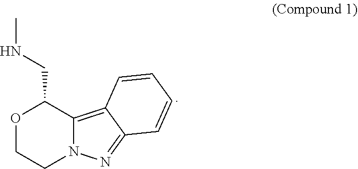 [SMILES: CNC[C@H]1OCCn2nc3ccccc3c21]

In some embodiments, provided are processes of preparing (R)-1-(3,4-dihydro-1H-[1,4]oxazino[4,3-b]indazol-1-yl)-N-methylmethanamine (Compound 1), or salts or crystalline forms thereof.

In some embodiments, provided are methods of using (R)-1-(3,4-dihydro-1H-[1,4]oxazino[4,3-b]indazol-1-yl)-N-methylmethanamine (Compound 1), or salts or crystalline forms thereof, in the treatment of CNS disorders.

In some embodiments, provided are pharmaceutical compositions comprising (R)-1-(3,4-dihydro-1H-[1,4]oxazino[4,3-b]indazol-1-yl)-N-methylmethanamine (Compound 1), or salts or crystalline forms thereof, as described herein, and one or more pharmaceutically acceptable excipients.

### BRIEF DESCRIPTION OF THE DRAWINGS

FIG. 1 shows an X-ray powder diffraction (XRPD) pattern of Compound 1 Tosylate, Form TA.

FIG. 2 shows a differential scanning calorimetry (DSC) thermogram of Compound 1 Tosylate, Form TA.

FIG. 3 shows a thermogravimetric analysis (TGA) thermogram of Compound 1 Tosylate, Form TA.

FIG. 4 shows a dynamic vapor sorption (DVS) isotherm of Compound 1 Tosylate, Form TA.

FIG. 5 shows an XRPD pattern of Compound 1 Besylate, Form BA.

FIG. 6 shows a DSC thermogram of Compound 1 Besylate, Form BA.

FIG. 7 shows a DVS isotherm of Compound 1 Besylate, Form BA.

FIG. 8 shows an XRPD pattern of Compound 1 Hydrochloride, Form HA.

FIG. 9 shows a DSC thermogram of Compound 1 Hydrochloride, Form HA.

FIG. 10 shows a TGA thermogram of Compound 1 Hydrochloride, Form HA.

FIG. 11 shows a DVS isotherm of Compound 1 Hydrochloride, Form HA.

FIG. 12 shows an XRPD pattern of Compound 1 Hydrochloride, Form HC.

FIG. 13 shows a DSC thermogram of Compound 1 Hydrochloride, Form HC.

FIG. 14 shows a TGA thermogram of Compound 1 Hydrochloride, Form HC.

FIG. 15 shows a DVS isotherm of Compound 1 Hydrochloride, Form HC.

FIG. 16 shows Raman spectra of Compound 1 Hydrochloride, Form HA, Form HB, Form HC, and Form HD.

FIG. 17 shows comparison XRPD spectra of Compound 1 Hydrochloride, Form HA, Form HC, and Form HD.

FIG. 18 shows a DSC thermogram of Compound 1 Hydrochloride of both Form HC and Form HD.

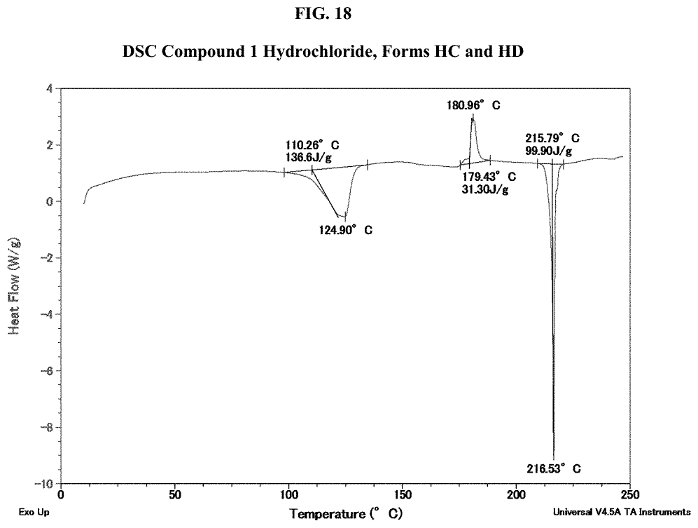

FIG. 19 shows a TGA thermogram of Compound 1 Hydrochloride of both Form HC and Form HD.

FIG. 20 shows an XRPD pattern of Compound 1 Fumarate, Form FA.

FIG. 21 shows a DSC thermogram of Compound 1 Fumarate, Form FA.

FIG. 22 shows a TGA thermogram of Compound 1 Fumarate, Form FA.

FIG. 23 shows a DVS isotherm of Compound 1 Fumarate, Form FA.

FIG. 24 shows an XRPD pattern of Compound 1 Fumarate, Form FB.

FIG. 25 shows a DSC thermogram of Compound 1 Fumarate, Form FB.

FIG. 26 shows a TGA thermogram of Compound 1 Fumarate, Form FB.

FIG. 27 shows a DVS isotherm of Compound 1 Fumarate, Form FB.

FIG. 28 shows an XRPD pattern of Compound 1 Phosphate, Form PA.

FIG. 29 shows a DSC thermogram of Compound 1 Phosphate, Form PA.

FIG. 30 shows a TGA thermogram of Compound 1 Phosphate, Form PA.

FIG. 31 shows the fluidic configuration of the Corning AFR reactor system.

FIG. 32 shows the fluidic configuration of the HX Exergy Tubular reactor system.

### DETAILED DESCRIPTION

The description herein sets forth details to provide an understanding of various embodiments of the invention, and is made with the understanding that the provided disclosures are an exemplification of the claimed subject matter without intending to limit the claims to specific embodiments. Accordingly, specific embodiments disclosed herein may be combined with other specific embodiments disclosed herein, including specific embodiments under various headings, which are provided for convenience and organization, but are not to be construed to limit the claims in any way.

All published documents cited herein are hereby incorporated by reference in their entirety.

### Definitions

Unless defined otherwise, all technical and scientific terms used herein have the same meaning as commonly understood by one of ordinary skill in the art.

As used herein, the singular forms “a”, “an” and “the” are intended to include the plural forms as well, unless the context clearly indicates otherwise.

As used herein, and unless otherwise specified, the term “about”, when used in connection with a numeric value or range of values which is provided to describe a particular solid form (e.g., a specific temperature or temperature range, such as describing a melting, dehydration, or glass transition; a mass change, such as a mass change as a function of temperature or humidity; a solvent or water content, in terms of, for example, mass or a percentage; or a peak position, such as in analysis by, for example, ¹³C NMR, DSC, TGA and XRPD), indicate that the value or range of values may deviate to an extent deemed reasonable to one of ordinary skill in the art while still describing the particular solid form. Specifically, the term “about”, when used in this context, indicates that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values while still describing the particular solid form. The term “about”, when used in reference to a degree 2-theta value refers to +/−0.3 degrees 2-theta or +/−0.2 degrees 2-theta.

As used herein, the phrase “alkali metal bicarbonate,” employed alone or in combination with other terms, refers to a base having formula M(HCO₃), wherein M refers to an alkali metal (e.g. lithium, sodium, or potassium). Example alkali metal bicarbonate include, but are not limited to, lithium bicarbonate, sodium bicarbonate, and potassium bicarbonate.

As used herein, the phrase “alkali metal alkoxide,” employed alone or in combination with other terms, refers to a base having formula M(O-alkyl), wherein M refers to an alkali metal (e.g. lithium, sodium, or potassium). Examples alkali metal alkoxide include, but are not limited to lithium alkoxide, sodium alkoxide, and potassium alkoxide.

As used herein, the terms “comprising” and “including” or grammatical variants thereof are to be taken as specifying the stated features, integers, steps or components but do not preclude the addition of one or more additional features, integers, steps, components or groups thereof. These terms encompass the term “consisting of”.

As used herein, the term “amorphous” or “amorphous form” is intended to mean that the substance, component, or product in question is not crystalline as determined, for instance, by XRPD or where the substance, component, or product in question, for example is not birefringent when viewed microscopically. For example, amorphous means essentially without regularly repeating arrangement of molecules or lacks the long range order of a crystal, i.e., amorphous form is non-crystalline. An amorphous form does not display a defined x-ray diffraction pattern with sharp maxima. In certain embodiments, a sample comprising an amorphous form of a substance can be substantially free of other amorphous forms and/or crystalline forms. For example, an amorphous substance can be identified by an XRPD spectrum having an absence of readily distinguishable reflections.

As used herein, the term “chemical purity” or “purity” refers to a measurement of purity compound. In some embodiments, the compound described herein can be isolated with a purity of at least about 80%, about 85%, about 90%, about 95%, about 96%, about 97%, about 98%, or about 99%. In some embodiments, the compound described herein can be isolated with an enantiomeric purity greater than about 90%. In some embodiments, the compound described herein can be isolated with an enantiomeric purity greater than about 95%. In some embodiments, the compound described herein can be isolated with an enantiomeric purity greater than about 99%. The measurement can be determined by methods well-known in the art, e.g., by elemental analysis, column chromatography, NMR spectroscopy, and the like.

As used herein, the terms “converting” with respect to changing an intermediate or starting reagent or material in a chemical reaction refers to subjecting the intermediate or starting reagent or material to the suitable reagents and conditions (e.g., temperature, time, solvent, etc.) to effect certain changes (e.g., breaking or formation of a chemical bond) to generate the desired product.

As used herein, the term “crystalline” or “crystalline form” refers to a crystalline solid form of a chemical compound, including, but not limited to, a single-component or multiple-component crystal form, e.g., including solvates, hydrates, clathrates, and a co-crystal. For example, crystalline means having a regularly repeating and/or ordered arrangement of molecules, and possessing a distinguishable crystal lattice. The term “crystalline form” is meant to refer to a certain lattice configuration of a crystalline substance. Different crystalline forms of the same substance typically have different crystalline lattices (e.g., unit cells), typically have different physical properties attributed to their different crystalline lattices, and in some instances, have different water or solvent content. The different crystalline lattices can be identified by solid state characterization methods such as by X-ray powder diffraction (XRPD). Other characterization methods such as differential scanning calorimetry (DSC), thermogravimetric analysis (TGA), dynamic vapor sorption (DVS), and the like further help identify the crystalline form as well as help determine stability and solvent/water content.

As used herein, the term “% crystallinity” or “crystalline purity,” means percentage of a crystalline form in a preparation or sample, which may contain other forms such as an amorphous form of the same compound, or at least one other crystalline form of the compound, or mixtures thereof. In some embodiments, the crystalline forms can be isolated with a purity of at least about 80%, about 85%, about 90%, about 95%, about 96%, about 97%, about 98%, or about 99%. In some embodiments, the crystalline forms can be isolated with a purity greater than about 90%. In some embodiments, the crystalline forms can be isolated with a purity greater than about 95%. In some embodiments, the crystalline forms can be isolated with a purity greater than about 99%.

As used herein, “delaying” development of a disorder mean to defer, hinder, slow, stabilize, and/or postpone development of the disorder. Delay can be of varying lengths of time, depending on the history of the disease and/or the individual being treated.

As used herein, the term “disorder” or specifically identified disorders disclosed herein, (e.g. CNS disorders) refer to the disorder as defined in the Diagnostic and Statistical Manual of Mental Disorders, Fifth Edition (DSM-5).

As used herein, the term “enantiomeric purity” refers to a measurement of purity for a chiral compound. In some embodiments, the compound described herein can be isolated with an enantiomeric purity of at least about 80%, about 85%, about 90%, about 95%, about 96%, about 97%, about 98%, or about 99%. In some embodiments, the compound described herein can be isolated with an enantiomeric purity greater than about 99%. In some embodiments, the compound described herein can be isolated with an enantiomeric purity greater than about 90%. In some embodiments, the compound described herein can be isolated with an enantiomeric purity greater than about 95%. The measurement can be determined by methods well-known in the art, e.g., by specific optical rotation, chiral column chromatography, NMR spectroscopy, and the like.

The term “hydrate,” as used herein, is meant to refer to a solid form (e.g., crystalline form) of Compound 1 and its salts that includes water. The water in a hydrate can be present in a stoichiometric amount with respect to the amount of salt in the solid, or can be present in varying amounts, such as can be found in connection with channel hydrates.

As used herein, the term “organic solvent” refers to carbon-based solvents (i.e., they contain carbon in their structure) that are employed to dissolve or disperse one or more compounds described herein. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

Suitable solvents can include halogenated solvents such as carbon tetrachloride, bromodichloromethane, dibromochloromethane, bromoform, chloroform, bromochloromethane, dibromomethane, butyl chloride, dichloromethane (methylene chloride), tetrachloroethylene, trichloroethylene, 1,1,1-trichloroethane, 1,1,2-trichloroethane, 1,1-dichloroethane, 2-chloropropane, I,I,I-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

Suitable ether solvents include dimethoxymethane, tetrahydrofuran, 1,3-dioxane, 1,4-dioxane, furan, tetrahydrofuran (THF), diethyl ether, ethylene glycol dimethyl ether, ethylene glycol diethyl ether, diethylene glycol dimethyl ether (diglyme), diethylene glycol diethyl ether, triethylene glycol dimethyl ether, anisole, tent-butyl methyl ether, mixtures thereof and the like.

Suitable protic solvents can include, by way of example and without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, iso-butyl alcohol, tent-butyl alcohol, 2-ethoxyethanol, diethylene glycol, 1-, 2-, or 3-pentanol, neo-pentyl alcohol, tent-pentyl alcohol, diethylene glycol monomethyl ether, diethylene glycol monoethyl ether, cyclohexanol, benzyl alcohol, phenol, or glycerol.

Suitable aprotic solvents can include, by way of example and without limitation, N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide.

Suitable hydrocarbon solvents include benzene, cyclohexane, pentane, hexane, toluene, cycloheptane, methylcyclohexane, heptane, ethylbenzene, m-, o-, or p-xylene, octane, indane, nonane, or naphthalene.

As used herein, the term “peak” or “characteristic peak” refers to a reflection having a relative height/intensity of at least about 3% of the maximum peak height/intensity.

As used herein, “pharmaceutically acceptable” or “physiologically acceptable” refer to compounds (e.g., solid forms), compositions, dosage forms and other materials, which are useful in preparing a pharmaceutical composition that is suitable for veterinary or human pharmaceutical use.

The term “pharmaceutically acceptable excipient” refers to a non-toxic binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccant, plasticizer, vehicle, disintegrant, or lubricant that does not destroy the pharmacological activity of the compound with which it is formulated. Pharmaceutically acceptable excipients that can be used in the compositions of this invention include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the term “potency” refers to a measure of compound activity expressed in terms of the amount required to produce an effect of given intensity. For example, if a reagent is 80% potent, then the reagent will provide at most an 80% yield, calculated on the basis of the reagent. Potency can be determined by, e.g., ¹H NMR or titration.

As used herein, “prevention” or “preventing” refers to a regimen that protects against the onset of the disorder such that the clinical symptoms of the disorder do not develop. Accordingly, “prevention” relates to administration of a therapy, including administration of a compound disclosed herein, to a subject before signs of the diseases are detectable in the subject (for example, administration of a compound disclosed herein to a subject in the absence of a detectable syndrome of the disorder). The subject may be at risk of developing the disorder. As used herein, an “at risk” subject is one who is at risk of developing a disorder to be treated. This may be shown, for example, by one or more risk factors, which are measurable parameters that correlate with development of a disorder and are known in the art.

Preparation of compounds can involve the protection and deprotection of various chemical groups. The need for protection and deprotection, and the selection of appropriate protecting groups (PG) can be readily determined by one skilled in the art. The chemistry of protecting groups can be found, for example, in Wuts and Greene, Greene's Protective Groups in Organic Synthesis, 4^{th} Ed., John Wiley & Sons: New York, 2006, which is incorporated herein by reference in its entirety. Preparation of compounds can also include leaving group (LG), which is a molecular fragment that leaves in bond cleavage. Leaving groups can be anions or neutral fragment and is able to stabilize the additional electron density that results from bond cleavage. Typical leaving groups are halides such as Cl, Br, and I, and sulfonate esters such as tosylate (TsO), triflate (TfO), mesylate (MsO), and the like.

As used herein, the term “reacting” is used as known in the art and generally refers to the bringing together of chemical reagents in such a manner so as to allow their interaction at the molecular level to achieve a chemical or physical transformation. In some embodiments, the reacting involves at least two reagents, wherein one or more molar equivalents of second reagent are used with respect to the first reagent. In some embodiments, the reacting step of a synthetic process may involve one or more substances in addition to the reagents such as solvent and/or a catalyst. The reacting steps of the processes described herein can be conducted for a time and under conditions suitable for preparing the identified product.

As used herein, the term “residence time” refers to the amount of time matter spends in a flow reactor system. In some embodiments, the residence time refers to the total amount of time from when Compound 1a enters the flow reactor to when it exits the reactor as incorporated into Compound 4a. In some embodiments, the residence time refers to the total amount of time from when Compound 3a enters the flow reactor to when it exits the reactor as incorporated into Compound 4a.

As used herein, the term “salt” refers to a substance that results from the combination of a compound and an acid or a base. For example, the free base Compound 1 can be combined with the desired acid in a solvent or in a melt to generate a salt of Compound 1. In some embodiments, acid addition salt of Compound 1 can be converted to a different acid addition salt by anion exchange. Salts of the invention which are prepared in a solvent system can be isolated by precipitation from the solvent. Precipitation and/or crystallization can be induced, for example, by evaporation, reduction of temperature, addition of anti-solvent, or combinations thereof In some embodiments, the term “compound” is used to refer to a salt of Compound 1. For example, Compound 1 Tosylate may be referred to as a salt or a compound.

As used herein, the term “solid form” refers to a compound provided herein in either an amorphous state or a crystalline state (e.g., crystalline form), whereby a compound provided herein in a crystalline state may optionally include solvent or water within the crystalline lattice, for example, to form a solvated or hydrated crystalline form. In some embodiments, the compound provided herein is in a crystalline state as described herein.

A “solvate” as used herein is formed by the interaction of a solvent and a compound.

As used herein, the term “subject,” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The “subject” may have independently been diagnosed with a disorder as defined herein, may currently be experiencing symptoms associated with disorders or may have experienced symptoms in the past, may be at risk of developing a disorder, or may be reporting one or more of the symptoms of a disorder, even though a diagnosis may not have been made. In some embodiments, the subject is a human who may have independently been diagnosed with a disorder as defined herein, may currently be experiencing symptoms associated with disorders or may have experienced symptoms in the past, may be at risk of developing a disorder, or may be reporting one or more of the symptoms of a disorder, even though a diagnosis may not have been made.

As used herein, the term “substantially” when referring to a characteristic figure of a crystal form, such as an XRPD pattern, a DSC thermogram, a TGA thermogram, or the like, means that a subject figure can be non-identical to the reference depicted herein, but it falls within the limits of experimental error and thus can be deemed as derived from the same crystal form as disclosed herein, as judged by a person of ordinary skill in the art. For example, the term “substantially” as used in the context of XRPD herein is meant to encompass variations disclosed herein (e.g., instrument variation, measurement variation, etc.).

As used herein, the term “substantially amorphous” means a majority of the weight of a sample or preparation (e.g., of a salt of Compound 1) is amorphous and the remainder of the sample is a crystalline form of the same compound. In some embodiments, a substantially amorphous sample has less than about 5% crystallinity (e.g., about 95% of the non-crystalline form of the same compound), preferably less than about 4% crystallinity (e.g., about 96% of the non-crystalline form of the same compound), more preferably less than about 3% crystallinity (e.g., about 97% of the non-crystalline form of the same compound), even more preferably less than about 2% crystallinity (e.g., about 98% of the non-crystalline form of the same compound), still more preferably less than about 1% crystallinity (e.g., about 99% of the non-crystalline form of the same compound), and most preferably about 0% crystallinity (e.g., about 100% of the non-crystalline form of the same compound). In some embodiments, the term “fully amorphous” means less than about 99% or about 0% crystallinity.

As used herein, the term “substantially crystalline,” means a majority of the weight of a sample or preparation (e.g., of a salt of Compound 1) is crystalline and the remainder of the sample is a non-crystalline form (e.g., amorphous form) of the same compound. In some embodiments, a substantially crystalline sample has at least about 95% crystallinity (e.g., about 5% of the non-crystalline form of the same compound), preferably at least about 96% crystallinity (e.g., about 4% of the non-crystalline form of the same compound), more preferably at least about 97% crystallinity (e.g., about 3% of the non-crystalline form of the same compound), even more preferably at least about 98% crystallinity (e.g., about 2% of the non-crystalline form of the same compound), still more preferably at least about 99% crystallinity (e.g., about 1% of the non-crystalline form of the same compound), and most preferably about 100% crystallinity (e.g., about 0% of the non-crystalline form of the same compound). In some embodiments, the term “fully crystalline” means at least about 99% or about 100% crystallinity.

The term “substantially isolated” is meant that the compound is at least partially or substantially separated from the environment in which it was formed or detected. Partial separation can include, e.g., a composition enriched in the compound, salts, hydrates, solvates, or solid forms provided herein. Substantial separation can include compositions containing at least about 50%, at least about 60%, at least about 70%, at least about 80%, at least about 90%, at least about 95%, at least about 97%, or at least about 99% by weight of the compound, salts, hydrates, solvates, or solid forms provided herein.

As used herein, the term “therapeutically effective amount” or “effective amount” refers to an amount that is effective to elicit the desired biological or medical response, including the amount of a compound that, when administered to a subject for treating a disorder, is sufficient to effect such treatment of the disorder. The effective amount will vary depending on the compound, the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, the terms “treatment,” “treat,” and “treating” refer to an approach for obtaining beneficial or desired results including, but not limited to, therapeutic benefit. Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder. In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life). In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “treatment-resistant depression,” which is also known as “treatment-refractory depression,” refers to major depressive disorder (MDD) situations where the subject shows inadequate responses to treatment with at least two antidepressants (e.g., standard antidepressant treatments that are commercially available). Inadequate response can be no response. Inadequate response can also be when the subject does not show full remission of symptoms, or when the physician or clinician does not deem the subject's response to be adequate. Treatment-resistant depression symptoms can range from mild to severe. Factors that can contribute to inadequate response include, but not limited to, early discontinuation of treatment, insufficient dosage of medication, patient noncompliance, misdiagnosis, and concurrent psychiatric disorders.

The following abbreviations may be used herein: aq. (aqueous); atm. (atmosphere(s)); Boc (t-butoxycarbonyl); br (broad); Cbz (carboxybenzyl); calc. (calculated); d (doublet); dd (doublet of doublets); DCM (dichloromethane); DIPEA (N,N-diisopropylethylamine); DMF (N,N-dimethylformamide); Et (ethyl); EtOAc (ethyl acetate); g (gram(s)); h (hour(s)); HCl (hydrochloric acid); Hz (hertz); J (coupling constant); m (multiplet); M (molar); MgSO₄ (magnesium sulfate); MS (Mass spectrometry); Me (methyl); MeCN (acetonitrile); MeOH (methanol); mg (milligram(s)); min. (minutes(s)); mL (milliliter(s)); mmol (millimole(s)); MsO (mesylate); NaHCO₃ (sodium bicarbonate); NaOH (sodium hydroxide); nM (nanomolar); NMR (nuclear magnetic resonance spectroscopy); N-methylpyrrolidone (NMP); TMEDA (tetramethylethylenediamine); TfO (trifluoromethanesulfonate; triflate); TsO (tosylate); Pd (palladium); Ph (phenyl); s (singlet); t (triplet or tertiary); TBS (tert-butyldimethylsilyl); tert (tertiary); Ts (p-toluenesulfonyl); tt (triplet of triplets); t-Bu (tert-butyl); TFA (trifluoroacetic acid); THF (tetrahydrofuran); μg (microgram(s)); μL (microliter(s)); μM (micromolar); wt % (weight percent).

### Salts and Crystalline Forms Thereof

Provided herein are salts of (R)-1-(3,4-dihydro-1H-[1,4]oxazino[4,3-b]indazol-1-yl)-N-methylmethanamine (Compound 1), and crystalline forms thereof. (R)-1-(3,4-dihydro-1H-[1,4]oxazino[4,3-b]indazol-1-yl)-N-methylmethanamine (Compound 1) has the structure:

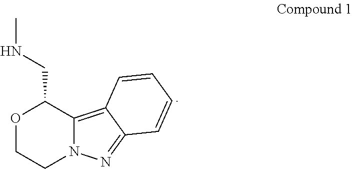 [SMILES: CNC[C@H]1OCCn2nc3ccccc3c21]

Compound 1 can also be represented as follows:

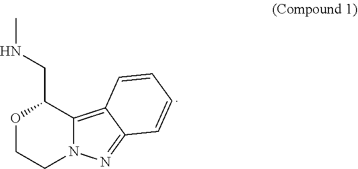 [SMILES: CNC[C@H]1OCCn2nc3ccccc3c21]

Compound 1 is described in PCT Publication No: WO2016/130790, the entirety of which is incorporated herein by reference.

Compound 1 ((R)-1-(3,4-dihydro-1H-[1,4]oxazino[4,3-b]indazol-1-yl)-N-methylmethanamine) is named or identified using other commonly recognized nomenclature systems. For example, the compound may be named or identified with common names, systematic names, or non-systematic names. The nomenclature systems that are commonly recognized in the art of chemistry include, but are not limited to, Chemical Abstract Service (CAS) and International Union of Pure and Applied Chemistry (IUPAC). The IUPAC name provided by ChemDraw Professional 16.0 has been used herein for Compound 1. Compound 1 has a CAS Registry Number of 1984744-86-6.

Compound 1 may be prepared as a salt. In some embodiments, Compound 1 may be prepared as a pharmaceutically acceptable salt. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, tosylates, xylenesulfonates, naphthalene-1-sulfonates, naphthalene-2-sulfonates, phenylacetates, phenylpropionates, phenylbutyrates, citrates, lactates, gamma-hydroxybutyrates, glycolates, tartrates, and mandelates. Lists of other suitable pharmaceutically acceptable salts are found in Remington: The Science and Practice of Pharmacy, 21st Edition, Lippincott Williams and Wilkins, Philadelphia, Pa., 2006.

In some embodiments, the salt is a toluenesulfonic acid salt of Compound 1. In some embodiments, the salt is a p-toluenesulfonic acid (4-methylbenzenesulfonic acid, 4-toluenesulfonic acid, PTSA, pTsOH) salt of Compound 1. The toluenesulfonic acid salt form of Compound 1 is referred to herein as “Compound 1 Tosylate.” An alternative name for the salt is (R)-1-(3,4-dihydro-1H-[1,4]oxazino[4,3-b]indazol-1-yl)-N-methylmethanamine tosylate.

In some embodiments, the salt is a benzenesulfonic acid salt of Compound 1. In some embodiments, benzenesulfonic acid salt form of Compound 1 is referred to herein as “Compound 1 Besylate.” Alternative names for the salt are (R)-1-(3,4-dihydro-1H-[1,4]oxazino[4,3-b]indazol-1-yl)-N-methylmethanamine benzenesulfonate and (R)-1-(3,4-dihydro-1H-[1,4]oxazino[4,3-b]indazol-1-yl)-N-methylmethanamine besylate.

In some embodiments, the salt is a hydrochloric acid salt of Compound 1. The hydrochloric acid salt form of Compound 1 is referred to herein as “Compound 1 Hydrochloride.” An alternative name for the salt is (R)-1-(3,4-dihydro-1H-[1,4]oxazino[4,3-b]indazol-1-yl)-N-methylmethanamine hydrochloride.

In some embodiments, the salt is a fumaric (trans-butenedioic) acid salt of Compound 1. The fumaric acid salt form of Compound 1 is referred to herein as “Compound 1 Fumarate.” An alternative name for the salt is (R)-1-(3,4-dihydro-1H-[1,4]oxazino[4,3-b]indazol-1-yl)-N-methylmethanamine fumarate.

In some embodiments, the salt is a phosphoric acid salt of Compound 1. The phosphoric acid salt form of Compound 1 is referred to herein as “Compound 1 Phosphate.” An alternative name for the salt is (R)-1-(3,4-dihydro-1H-[1,4]oxazino[4,3-b]indazol-1-yl)-N-methylmethanamine phosphate.

The salts described herein can have about half, about 1, about 2, about 3 equivalents, etc. of acid to Compound 1. In some embodiments, the salts described herein comprises about half equivalent of acid to Compound 1. In some embodiments, the salts described herein comprise about 1 equivalent of acid to Compound 1. In some embodiments, the salts described herein comprise about 2 equivalents of acid to Compound 1. In some embodiments, the salts described herein comprise about 3 equivalents of acid to Compound 1. A person skilled in the art would recognize that there is an equilibrium between the acid and Compound 1 in which the protons may reside, which depends on the conditions (e.g., solvents, temperature, etc.) and the strength of the acids. For example, in some conditions, the acid becomes a counter-anion by losing one or more protons to Compound 1, and Compound 1 becomes a counter-cation. In some conditions, the protons of the acids may form a weak interaction with the basic sites of Compound 1 and thus, the protons are shared between the acid and Compound 1.

The salts described herein can have less than about 1, about 1, about 2, about 3, about 4, about 5, or greater than about 6 equivalents of solvent or hydrate to the salt. In some embodiments, the salts described have less than about 1 equivalent of solvent or hydrate to the salt. In some embodiments, the salts described have less than about 1 equivalent of hydrate to the salt. In some embodiments, the salts described have about 2 equivalents of solvent or hydrate to the salt. In some embodiments, the salts described have about 2 equivalent of hydrate to the salt. In some embodiments, the salts described have about 3 equivalents of solvent or hydrate to the salt. In some embodiments, the salts described have about 3 equivalents of hydrate to the salt.

In some embodiments, the salts described herein are anhydrous.

Salts of Compound 1 can be isolated as one or more crystalline forms. Different crystalline forms of the same substance may have different bulk properties relating to, for example, hygroscopicity, solubility, stability, and the like. Crystalline forms with high melting points may have good thermodynamic stability, which may be advantageous in prolonging shelf-life drug formulations containing the crystalline form. Crystalline forms with lower melting points may be less thermodynamically stable, but may be advantageous in having increased water solubility, which may translate to increased drug bioavailability. Crystalline forms that are weakly hygroscopic may be desirable for stability to heat or humidity and may be resistant to degradation during long storage. The crystalline forms described herein have many advantages, for example they have desirable properties. Moreover, the crystalline forms disclosed herein may be useful for improving the performance characteristics of a pharmaceutical product such as dissolution profile, shelf-life and bioavailability.

Different crystalline forms of a particular substance, such as Compound 1 as described herein, can include both anhydrous forms of that substance and solvated/hydrated forms of that substance, where each of the anhydrous forms and solvated/hydrated forms are distinguished from each other by different XRPD patterns, or other solid state characterization methods, thereby signifying different crystalline lattices. In some instances, a single crystalline form (e.g., identified by a unique XRPD pattern) can have variable water or solvent content, where the lattice remains substantially unchanged (as does the XRPD pattern) despite the compositional variation with respect to water and/or solvent.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, filters used, the sample mounting procedure, and the particular instrument employed. In some instances, new peaks can be observed or existing peaks may disappear, depending on the type of the machine or the settings (for example, whether a Ni filter is used or not). Moreover, instrument variation and other factors can affect the 2-theta (2θ) values. Thus, peak assignments, such as those reported herein, can vary by plus or minus (±) about 0.2° (2-theta) or about 0.3° (2-theta).

In the same way, temperature readings in connection with DSC, TGA, or other thermal experiments can vary about ±3° C. depending on the instrument, particular settings, sample preparation, etc. Accordingly, a crystalline form reported herein having a DSC thermogram “substantially” as shown in any of the Figures is understood to accommodate such variation.

Crystalline forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates such as, e.g., on polymers, recrystallization in the presence of additives, such as, e.g., co-crystal counter-molecules, desolvation, dehydration, rapid evaporation, rapid cooling, slow cooling, vapor diffusion, sublimation, exposure to moisture, grinding and solvent-drop grinding.

Compound 1 and its salts can be prepared in batches referred to as batches, samples, or preparations. The batches, samples, or preparations can include Compound 1 and its salts in any of the crystalline or non-crystalline forms described herein, including hydrated and non-hydrated forms, and mixtures thereof.

Compounds provided herein (e.g., salts of Compound 1) can also include all isotopes of atoms occurring in the intermediates or final compounds. Isotopes include those atoms having the same atomic number but different mass numbers. For example, isotopes of hydrogen include tritium and deuterium. One or more constituent atoms of the compounds provided herein can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7 or 8 deuterium atoms. Synthetic methods for including isotopes into organic compounds are known in the art. Examples of isotopes that can be incorporated into the compounds disclosed herein include isotopes of hydrogen, carbon, nitrogen, oxygen, phosphorus, fluorine, chlorine, and iodine (e.g., ²H, ³H, ¹¹C, ¹³C, ¹⁴C, ¹³N, ¹⁵N, ¹⁵O, ¹⁸O, ³¹P, ³²P, ³⁵S, ¹⁸F, ³⁶Cl, ¹²³I, and ¹²⁵I).

In some embodiments, Compound 1 or its salts and crystalline forms thereof are substantially isolated.

Compound 1 can be observed and/or isolated as various salt forms and polymorphs thereof, including, e.g., tosylate salt (e.g., Form TA), besylate salt (e.g., Form BA), hydrochloride salt (e.g., Form HA, Form HB, Form HC, and Form HD), fumarate salt (e.g., Form FA and Form FB), and phosphate (e.g., Form PA).

### Compound 1 Tosylate

Provided herein is a p-toluenesulfonic acid salt of Compound 1: (R)-1-(3,4-dihydro-1H-[1,4]oxazino[4,3-b]indazol-1-yl)-N-methylmethanamine tosylate (Compound 1 Tosylate), which is described herein, including the Examples. In some embodiments, the salt is anhydrous. The salt can be crystalline. In some embodiments, provided is anhydrous (R)-1-(3,4-dihydro-1H-[1,4]oxazino[4,3-b]indazol-1-yl)-N-methylmethanamine tosylate (anhydrous Compound 1 Tosylate). In some embodiments, provided is crystalline (R)-1-(3,4-dihydro-1H-[1,4]oxazino[4,3-b]indazol-1-yl)-N-methylmethanamine tosylate (crystalline Compound 1 Tosylate).

In some embodiments, provided is crystalline (R)-1-(3,4-dihydro-1H-[1,4]oxazino[4,3-b]indazol-1-yl)-N-methylmethanamine tosylate Form TA (Compound 1 Tosylate Form TA).

In some embodiments, Compound 1 Tosylate Form TA has at least one characteristic XRPD peak selected from about 5.7, about 11.3, and about 16.4 degrees 2-theta. In some embodiments, Compound 1 Tosylate Form TA has at least one characteristic XRPD peak selected from about 5.7, about 11.3, about 16.4, about 16.9, and about 22.6 degrees 2-theta.

In some embodiments, Compound 1 Tosylate Form TA has at least two characteristic XRPD peaks selected from about 5.7, about 11.3, about 16.4, about 16.9, about 18.3, about 22.6, and about 28.4 degrees 2-theta.

In some embodiments, Compound 1 Tosylate Form TA has at least three characteristic XRPD peaks selected from about 5.7, about 11.3, about 16.4, about 16.9, about 18.3, about 22.6, and about 28.4 degrees 2-theta.

In some embodiments, Compound 1 Tosylate Form TA has an XRPD pattern comprising degree 2θ-reflections at 5.7°, 11.3°, and 16.4° 2θ, each of which is ±0.2° 2θ. In some embodiments, Compound 1 Tosylate Form TA has an XRPD pattern comprising degree 2θ-reflections at 5.7°, 11.3°, 16.4°, and 16.9° 2θ, each of which is ±0.2° 2θ. In some embodiments, Compound 1 Tosylate Form TA has an XRPD pattern comprising degree 2θ-reflections at 5.7°, 11.3°, 16.4°, 16.9°, and 22.6° 2θ, each of which is ±0.2° 2θ. In some embodiments, Compound 1 Tosylate Form TA has an XRPD pattern comprising degree 2θ-reflections at 5.7°, 11.3°, 16.4°, 16.9°, 22.6° and 28.4° 2θ, each of which is ±0.2° 2θ. In some embodiments, Compound 1 Tosylate Form TA has an XRPD pattern comprising degree 2θ-reflections at 5.7°, 11.3°, 16.4°, 16.9°, 18.3°, 22.6°, and 28.4° 2θ, each of which is ±0.2° 2θ. In some embodiments, Compound 1 Tosylate Form TA has an XRPD pattern comprising degree 2θ-reflections at 5.7°, 11.3°, 16.4°, 16.9°, 18.3°, 22.6°, 28.4°, and 34.2° 2θ, each of which is ±0.2° 2θ.

In some embodiments, Compound 1 Tosylate Form TA has an XRPD pattern comprising degree 2θ-reflections at 5.7°, 11.3°, and 16.4° 2θ, each of which is ±0.2° 2θ, and at least one degree 2θ-reflection selected from 16.9°, 18.3°, 22.6°, 28.4°, and 34.2° 2θ, each of which is ±0.2° 2θ. In some embodiments, Compound 1 Tosylate Form TA has an XRPD pattern comprising degree 2θ-reflections at 5.7°, 11.3°, and 16.4° 2θ, each of which is ±0.2° 2θ, and at least two degree 2θ-reflections selected from 16.9°, 18.3°, 22.6°, 28.4°, and 34.2° 2θ, each of which is ±0.2° 2θ. In some embodiments, Compound 1 Tosylate Form TA has an XRPD pattern comprising degree 2θ-reflections at 5.7°, 11.3°, and 16.4° 2θ, each of which is ±0.2° 2θ, and at least three degree 2θ-reflections selected from 16.9°, 18.3°, 22.6°, 28.4°, and 34.2° 2θ, each of which is ±0.2° 2θ. In some embodiments, Compound 1 Tosylate Form TA has an XRPD pattern comprising degree 2θ-reflections at 5.7°, 11.3°, and 16.4° 2θ, each of which is ±0.2° 2θ, and at least four degree 2θ-reflections selected from 16.9°, 18.3°, 22.6°, 28.4°, and 34.2° 2θ, each of which is ±0.2° 2θ.

In some embodiments, Compound 1 Tosylate Form TA has an XRPD pattern comprising degree 2θ-reflections at least at one degree 2θ-reflection identified in Table 4. In some embodiments, Compound 1 Tosylate Form TA has an XRPD pattern comprising degree 2θ-reflections at least at two degree 2θ-reflections identified in Table 4. In some embodiments, Compound 1 Tosylate Form TA has an XRPD pattern comprising degree 2θ-reflections at least at three degree 2θ-reflections identified in Table 4. In some embodiments, Compound 1 Tosylate Form TA has an XRPD pattern comprising degree 2θ-reflections at least at four degree 2θ-reflections identified in Table 4. In some embodiments, Compound 1 Tosylate Form TA has an XRPD pattern comprising degree 2θ-reflections at least at five degree 2θ-reflections identified in Table 4. In some embodiments, Compound 1 Tosylate Form TA has an XRPD pattern comprising degree 2θ-reflections at least at six degree 2θ-reflections identified in Table 4. In some embodiments, Compound 1 Tosylate Form TA has an XRPD pattern comprising degree 2θ-reflections at least at seven degree 2θ-reflections identified in Table 4. In some embodiments, Compound 1 Tosylate Form TA has an XRPD pattern comprising degree 2θ-reflections at least at eight degree 2θ-reflections identified in Table 4.

In some embodiments, Compound 1 Tosylate Form TA has an XRPD pattern comprising degree 2θ-reflections at least at one peak identified in Table 4. In some embodiments, Compound 1 Tosylate Form TA has an XRPD pattern comprising degree 2θ-reflections at least at two peaks identified in Table 4. In some embodiments, Compound 1 Tosylate Form TA has an XRPD pattern comprising degree 2θ-reflections at least at three peaks identified in Table 4. In some embodiments, Compound 1 Tosylate Form TA has an XRPD pattern comprising degree 2θ-reflections at least at four peaks identified in Table 4. In some embodiments, Compound 1 Tosylate Form TA has an XRPD pattern comprising peaks at least at five degree 2θ-reflections identified in Table 4. In some embodiments, Compound 1 Tosylate Form TA has an XRPD pattern comprising peaks at least at six degree 2θ-reflections identified in Table 4. In some embodiments, Compound 1 Tosylate Form TA has an XRPD pattern comprising degree 2θ-reflections at least at seven peaks identified in Table 4. In some embodiments, Compound 1 Tosylate Form TA has an XRPD pattern comprising degree 2θ-reflections at least at eight peaks identified in Table 4.

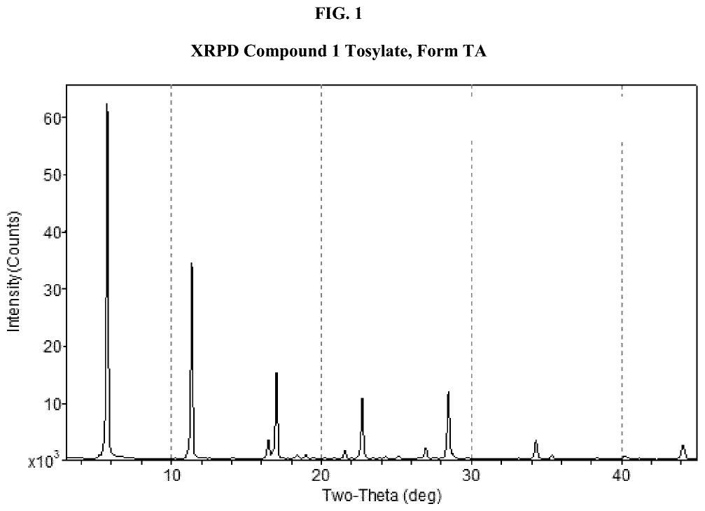

In some embodiments, Compound 1 Tosylate Form TA has an XRPD pattern with characteristic peaks as substantially shown in FIG. 1.

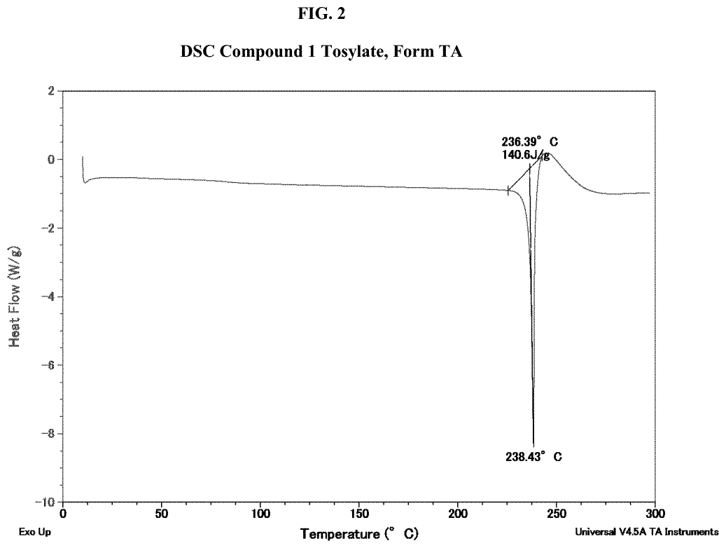

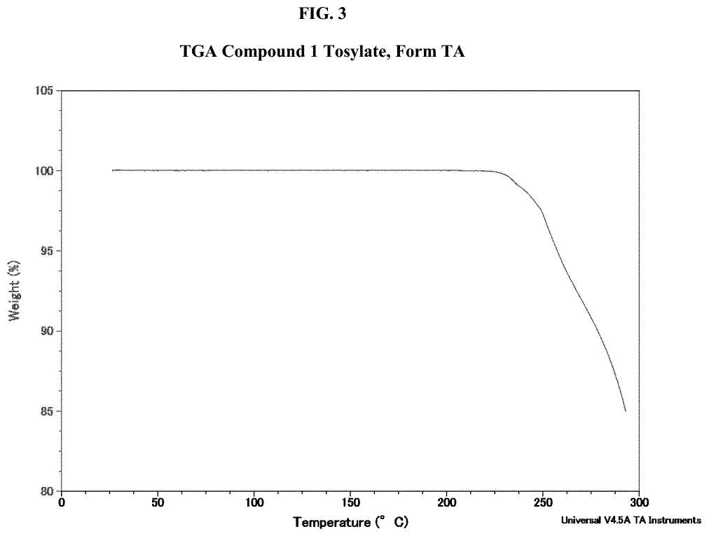

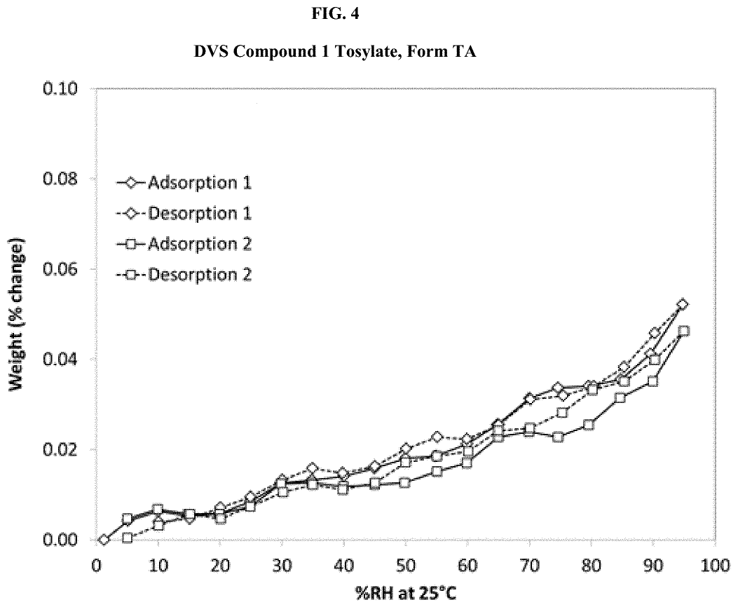

In some embodiments, Compound 1 Tosylate Form TA has an endotherm peak at a temperature of about 238° C. In some embodiments, Compound 1 Tosylate Form TA has a DSC thermogram substantially as depicted in FIG. 2. In some embodiments, Compound 1 Tosylate Form TA has a DVS isotherm substantially as depicted in FIG. 4. In some embodiments, Compound 1 Tosylate Form TA has a TGA thermogram substantially as depicted in FIG. 3.

In some embodiments, Compound 1 Tosylate Form TA has at least one characteristic XRPD peak selected from about 5.7, about 11.3, and about 16.4 degrees 2-theta; and an endotherm peak at a temperature of about 238° C. In some embodiments, Compound 1 Tosylate Form TA has a DSC thermogram substantially as depicted in FIG. 2. In some embodiments, Compound 1 Tosylate Form TA has at least one characteristic XRPD peak selected from about 5.7, about 11.3, and about 16.4 degrees 2-theta; and a DVS isotherm substantially as depicted in FIG. 4. In some embodiments, Compound 1 Tosylate Form TA has at least one characteristic XRPD peak selected from about 5.7, about 11.3, and about 16.4 degrees 2-theta; and a TGA thermogram substantially as depicted in FIG. 3.

In some embodiments, Compound 1 Tosylate Form TA can be isolated with a crystalline purity of at least about 80%, about 85%, about 90%, about 95%, about 96%, about 97%, about 98%, or about 99%. In some embodiments, Compound 1 Tosylate Form TA can be isolated with a crystalline purity greater than about 99%. In some embodiments, Compound 1 Tosylate Form TA can be isolated with a crystalline purity greater than about 99.9%.

Provided herein is also a process for preparing crystalline Compound 1 Tosylate, comprising precipitating Compound 1 Tosylate from a solution comprising Compound 1, toluenesulfonic acid, and S1, wherein S1 is a solvent. In some embodiments, S1 comprises chloroform, methanol, acetone, water, toluene, acetonitrile, or a mixture thereof.

In some embodiments, provided is Compound 1 Tosylate Form TA prepared by isolating Compound 1 Tosylate Form TA from a mixture of Compound 1, toluenesulfonic acid, and S1, wherein S1 is a solvent. In some embodiments, provided is Compound 1 Tosylate Form TA prepared by isolating Compound 1 Tosylate Form TA from a mixture of Compound 1, toluenesulfonic acid, and S1, wherein S1 comprises chloroform, methanol, acetone, water, toluene, acetonitrile, or a mixture thereof.

In some embodiments, the process to prepare crystalline Compound 1 Tosylate, comprises:
i. adding an alkali metal bicarbonate (e.g., sodium bicarbonate) in a first solvent (e.g., chloroform) to a solution of Compound 1 in water;
ii. separating the organic layer from the aqueous layer produced in (i);
iii. concentrating the organic layer of (ii) to produce a first oil;
iv. adding toluenesulfonic acid (e.g., toluenesulfonic acid monohydrate) and a second solvent (e.g., methanol) to the first oil;
v. concentrating the solution of (iv) to produce a second oil;
vi. suspending the second oil with acetone to produce crystalline precipitate of Compound 1 Tosylate; and
vii. isolating the crystalline precipitate of (vi).

### Compound 1 Besylate

Provided herein is a benzenesulfonic acid salt of Compound 1: (R)-1-(3,4-dihydro-1H-[1,4]oxazino[4,3-b]indazol-1-yl)-N-methylmethanamine benzenesulfonate, which is described herein, including the Examples. In some embodiments, the salt is anhydrous. The salt can be crystalline. In some embodiments, provided is anhydrous (R)-1-(3,4-dihydro-1H-[1,4]oxazino[4,3-b]indazol-1-yl)-N-methylmethanamine benzenesulfonate (anhydrous Compound 1 Besylate). In some embodiments, provided is crystalline (R)-1-(3,4-dihydro-1H-[1,4]oxazino[4,3-b]indazol-1-yl)-N-methylmethanamine benzenesulfonate (crystalline Compound 1 Besylate).

In some embodiments, provided is crystalline (R)-1-(3,4-dihydro-1H-[1,4]oxazino[4,3-b]indazol-1-yl)-N-methylmethanamine benzenesulfonate Form BA (Compound 1 Besylate Form BA).

In some embodiments, Compound 1 Besylate Form BA has at least one characteristic XRPD peak selected from about 5.9, about 11.8, about 17.1, and about 17.3 degrees 2-theta. In some embodiments, Compound 1 Besylate Form BA has at least one characteristic XRPD peak selected from about 5.9, about 11.8, about 17.1, about 17.3, about 18.2, about 19.2, and about 19.4 degrees 2-theta.

In some embodiments, Compound 1 Besylate Form BA has at least two characteristic XRPD peaks selected from about 5.9, about 11.8, about 17.1, about 17.3, about 18.2, about 19.2, about 19.4, about 22.8, about 23.8, about 24.4, and about 25.7 degrees 2-theta.

In some embodiments, Compound 1 Besylate Form BA has at least three characteristic XRPD peaks selected from about 5.9, about 11.8, about 17.1, about 17.3, about 18.2, about 19.2, about 19.4, about 22.8, about 23.8, about 24.4, and about 25.7 degrees 2-theta.

In some embodiments, Compound 1 Besylate Form BA has an XRPD pattern comprising degree 2θ-reflections at 5.9°, 11.8°, 17.1°, and 17.3° 2θ, each of which is ±0.2° 2θ. In some embodiments, Compound 1 Besylate Form BA has an XRPD pattern comprising degree 2θ-reflections at 5.9°, 11.8°, 17.1°, 17.3°, 18.2°, 19.2°, and 19.4° 2θ, each of which is ±0.2° 2θ. In some embodiments, Compound 1 Besylate Form BA has an XRPD pattern comprising degree 2θ-reflections at 5.9°, 11.8°, 17.1°, 17.3°, 18.2°, 19.2°, 19.4°, 22.8°, 23.8°, 24.4°, and 25.7°, each of which is ±0.2° 2θ. In some embodiments, Compound 1 Besylate Form BA has an XRPD pattern comprising degree 2θ-reflections at 5.9°, 11.8°, 17.1°, 17.3°, 18.2°, 19.2°, 19.4°, 22.8°, 23.8°, 24.4°, and 25.7°, each of which is ±0.2° 2θ.

In some embodiments, Compound 1 Besylate Form BA has an XRPD pattern comprising degree 2θ-reflections at 5.9°, 11.8°, 17.1°, and 17.3° 2θ, each of which is ±0.2° 2θ, and at least one degree 2θ-reflections selected from 18.2°, 19.2°, 19.4°, 22.8°, 23.8°, 24.4°, and 25.7°, each of which is ±0.2° 2θ. In some embodiments, Compound 1 Besylate Form BA has an XRPD pattern comprising degree 2θ-reflections at 5.9°, 11.8°, 17.1°, and 17.3° 2θ, each of which is ±0.2° 2θ, and at least two degree 2θ-reflections selected from 18.2°, 19.2°, 19.4°, 22.8°, 23.8°, 24.4°, and 25.7°, each of which is ±0.2° 2θ. In some embodiments, Compound 1 Besylate Form BA has an XRPD pattern comprising degree 2θ-reflections at 5.9°, 11.8°, 17.1°, and 17.3° 2θ, each of which is ±0.2° 2θ, and at least three degree 2θ-reflections selected from 18.2°, 19.2°, 19.4°, 22.8°, 23.8°, 24.4°, and 25.7°, each of which is ±0.2° 2θ. In some embodiments, Compound 1 Besylate Form BA has an XRPD pattern comprising degree 2θ-reflections at 5.9°, 11.8°, 17.1°, and 17.3° 2θ, each of which is ±0.2° 2θ, and at least four degree 2θ-reflections selected from 18.2°, 19.2°, 19.4°, 22.8°, 23.8°, 24.4°, and 25.7°, each of which is ±0.2° 2θ.

In some embodiments, Compound 1 Besylate Form BA has an XRPD pattern comprising degree 2θ-reflections at least at one degree 2θ-reflection identified in Table 5. In some embodiments, Compound 1 Besylate Form BA has an XRPD pattern comprising degree 2θ-reflections at least at two degree 2θ-reflections identified in Table 5. In some embodiments, Compound 1 Besylate Form BA has an XRPD pattern comprising degree 2θ-reflections at least at three degree 2θ-reflections identified in Table 5. In some embodiments, Compound 1 Besylate Form BA has an XRPD pattern comprising degree 2θ-reflections at least at four degree 2θ-reflections identified in Table 5. In some embodiments, Compound 1 Besylate Form BA has an XRPD pattern comprising degree 2θ-reflections at least at five degree 2θ-reflections identified in Table 5. In some embodiments, Compound 1 Besylate Form BA has an XRPD pattern comprising degree 2θ-reflections at least at six degree 2θ-reflections identified in Table 5. In some embodiments, Compound 1 Besylate Form BA has an XRPD pattern comprising degree 2θ-reflections at least at seven degree 2θ-reflections identified in Table 5. In some embodiments, Compound 1 Besylate Form BA has an XRPD pattern comprising degree 2θ-reflections at least at eight degree 2θ-reflections identified in Table 5.

In some embodiments, Compound 1 Besylate Form BA has an XRPD pattern comprising degree 2θ-reflections at least at one peak identified in Table 5. In some embodiments, Compound 1 Besylate Form BA has an XRPD pattern comprising degree 2θ-reflections at least at two peaks identified in Table 5. In some embodiments, Compound 1 Besylate Form BA has an XRPD pattern comprising degree 2θ-reflections at least at three peaks identified in Table 5. In some embodiments, Compound 1 Besylate Form BA has an XRPD pattern comprising degree 2θ-reflections at least at four peaks identified in Table 5. In some embodiments, Compound 1 Besylate Form BA has an XRPD pattern comprising degree 2θ-reflections at least at five peaks identified in Table 5. In some embodiments, Compound 1 Besylate Form BA has an XRPD pattern comprising degree 2θ-reflections at least at six peaks identified in Table 5. In some embodiments, Compound 1 Besylate Form BA has an XRPD pattern comprising degree 2θ-reflections at least at seven peaks identified in Table 5. In some embodiments, Compound 1 Besylate Form BA has an XRPD pattern comprising degree 2θ-reflections at least at eight peaks identified in Table 5.

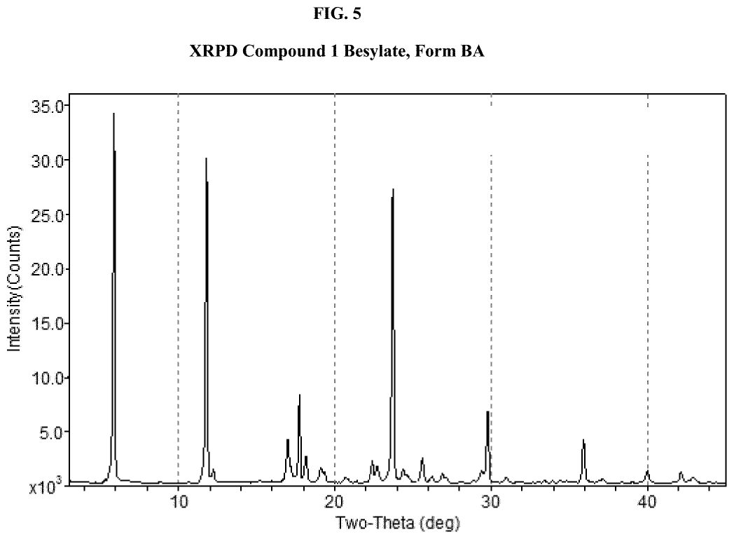

In some embodiments, Compound 1 Besylate Form BA has an XRPD pattern with characteristic peaks as substantially shown in FIG. 5.

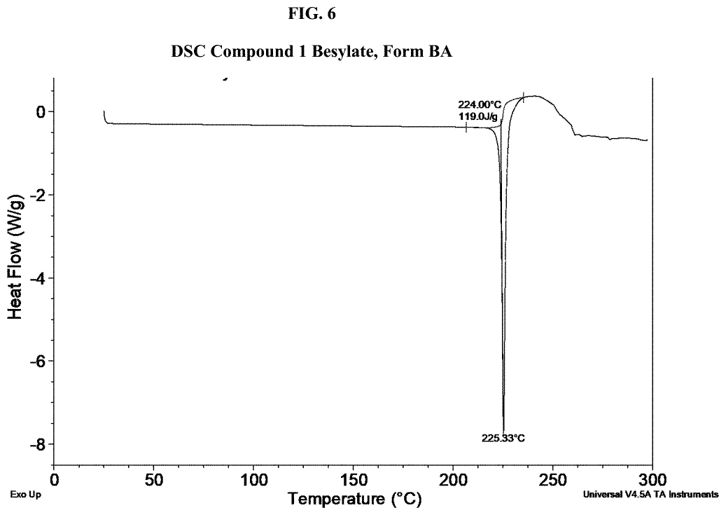

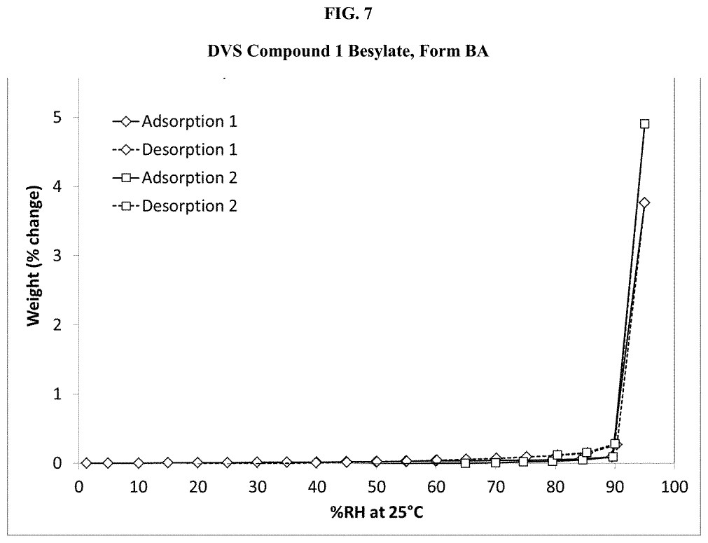

In some embodiments, Form BA has an endotherm peak at a temperature of about 225° C. In some embodiments, Form BA has a DSC thermogram substantially as depicted in FIG. 6. In some embodiments, Form BA has a DVS isotherm substantially as depicted in FIG. 7.

In some embodiments, Compound 1 Besylate Form BA has at least one characteristic XRPD peak selected from about 5.9, about 11.8, about 17.1, and about 17.3 degrees 2-theta; and an endotherm peak at a temperature of about 225° C. In some embodiments, Compound 1 Besylate Form BA has at least one characteristic XRPD peak selected from about 5.9, about 11.8, about 17.1, and about 17.3 degrees 2-theta; and a DSC thermogram substantially as depicted in FIG. 6. In some embodiments, Compound 1 Besylate Form BA has at least one characteristic XRPD peak selected from about 5.9, about 11.8, about 17.1, and about 17.3 degrees 2-theta; and a DVS isotherm substantially as depicted in FIG. 7.

In some embodiments, Compound 1 Besylate Form BA can be isolated with a purity of at least about 80%, about 85%, about 90%, about 95%, about 96%, about 97%, about 98%, or about 99%. In some embodiments, Compound 1 Besylate Form BA can be isolated with a purity greater than about 99%. In some embodiments, Compound 1 Besylate Form BA can be isolated with a purity greater than about 99.9%.

Provided herein is also a process for preparing crystalline Compound 1 Besylate comprising precipitating Compound 1 Besylate from a solution comprising Compound 1, benzensulfonic acid, and S2, wherein S2 is a solvent. In some embodiments, S2 comprises acetone.

In some embodiments, provided is Compound 1 Besylate Form BA prepared by isolating Compound 1 Besylate Form BA from a mixture of Compound 1, benzenesulfonic acid, and S2, wherein S2 is a solvent. In some embodiments, provided is Compound 1 Besylate Form BA prepared by isolating Compound 1 Besylate Form BA from a mixture of Compound 1, benzenesulfonic acid, and S2, wherein S2 comprises acetone.

In some embodiments, the process to prepare crystalline Compound 1 Besylate, comprises:
i. combining benzenesulfonic acid (e.g., benzenesulfonic acid monohydrate) and Compound 1;
ii. adding a solvent (e.g., acetone) to the combination of benzenesulfonic acid and Compound 1 to provide a mixture;
iii. shaking the mixture of (ii) in an incubator at about 40° C. to about 80° C. (e.g., about 60° C.) for about 1 to about 4 hours (e.g., about 2 hours);
iv. storing the mixture of (iii) at room temperature for about 5 to 20 hours to produce crystalline precipitate of Compound 1 Besylate; and
v. isolating the crystalline precipitate of (iv).

### Compound 1 Hydrochloride

Provided herein is a hydrochloric acid salt of Compound 1: (R)-1-(3,4-dihydro-1H-[1,4]oxazino[4,3-b]indazol-1-yl)-N-methylmethanamine hydrochloride, which is described herein, including the Examples. In some embodiments, the salt is a hydrate. The salt can be crystalline. In some embodiments, provided is crystalline (R)-1-(3,4-dihydro-1H-[1,4]oxazino[4,3-b]indazol-1-yl)-N-methylmethanamine hydrochloride (crystalline Compound 1 Hydrochloride).

In some embodiments, provided is crystalline (R)-1-(3,4-dihydro-1H-[1,4]oxazino[4,3-b]indazol-1-yl)-N-methylmethanamine hydrochloride Form HA (Compound 1 Hydrochloride Form HA). In some embodiments, Compound 1 Hydrochloride Form HA is a di-hydrochloride. In some embodiments, provided is a (R)-1-(3,4-dihydro-1H-[1,4]oxazino[4,3-b]indazol-1-yl)-N-methylmethanamine hydrochloride hydrate (Compound 1 Hydrochloride hydrate). For example, Compound 1 Hydrochloride Form HA is a mono-hydrate

In some embodiments, Compound 1 Hydrochloride Form HA has at least one characteristic XRPD peak selected from about 7.8, about 10.2, and about 13.4 degrees 2-theta. In some embodiments, Compound 1 Hydrochloride Form HA has at least one characteristic XRPD peak selected from about 7.8, about 10.2, about 13.4, about 15.6, about 15.9, and about 17.3 degrees 2-theta.

In some embodiments, Compound 1 Hydrochloride Form HA has at least two characteristic XRPD peaks selected from about 7.8, about 10.2, about 13.4, about 15.6, about 15.9, about 17.3, about 21.1, about 21.8, about 22.7, about 23.7, about 27.3, about 27.6, and about 29.7 degrees 2-theta.

In some embodiments, Compound 1 Hydrochloride Form HA has at least three characteristic XRPD peaks selected from about 7.8, about 10.2, about 13.4, about 15.6, about 15.9, about 17.3, about 21.1, about 21.8, about 22.7, about 23.7, about 27.3, about 27.6, and about 29.7 degrees 2-theta.

In some embodiments, Compound 1 Hydrochloride Form HA has an XRPD pattern comprising degree 2θ-reflections at 7.8°, 10.2°, and 13.4° 2θ, each of which is ±0.2° 2θ. In some embodiments, Compound 1 Hydrochloride Form HA has an XRPD pattern comprising degree 2θ-reflections at 7.8°, 10.2°, 13.4°, 15.6°, 15.9°, and 17.3° 2θ, each of which is ±0.2° 2θ. In some embodiments, Compound 1 Hydrochloride Form HA has an XRPD pattern comprising degree 2θ-reflections at 7.8°, 10.2°, 13.4°, 15.6°, 15.9°, 17.3°, 21.1°, 21.8°, 22.7°, 23.7°, 27.3°, 27.6°, and 29.7° 2θ, each of which is ±0.2° 2θ.

In some embodiments, Compound 1 Hydrochloride Form HA has an XRPD pattern comprising degree 2θ-reflections at 7.8°, 10.2°, and 13.4° 2θ, each of which is ±0.2° 2θ, and at least one degree 2θ-reflection selected from 15.6°, 15.9°, 17.3°, 21.1°, 21.8°, 22.7°, 23.7°, 27.3°, 27.6°, and 29.7° 2θ, each of which is ±0.2° 2θ. In some embodiments, Compound 1 Hydrochloride Form HA has an XRPD pattern comprising degree 2θ-reflections at 7.8°, 10.2°, and 13.4° 2θ, each of which is ±0.2° 2θ, and at least two degree 2θ-reflections selected from 15.6°, 15.9°, 17.3°, 21.1°, 21.8°, 22.7°, 23.7°, 27.3°, 27.6°, and 29.7° 2θ, each of which is ±0.2° 2θ. In some embodiments, Compound 1 Hydrochloride Form HA has an XRPD pattern comprising degree 2θ-reflections at 7.8°, 10.2°, and 13.4° 2θ, each of which is ±0.2° 2θ, and at least three degree 2θ-reflections selected from 15.6°, 15.9°, 17.3°, 21.1°, 21.8°, 22.7°, 23.7°, 27.3°, 27.6°, and 29.7° 2θ, each of which is ±0.2° 2θ. In some embodiments, Compound 1 Hydrochloride Form HA has an XRPD pattern comprising degree 2θ-reflections at 7.8°, 10.2°, and 13.4° 2θ, each of which is ±0.2° 2θ, and at least four degree 2θ-reflections selected from 15.6°, 15.9°, 17.3°, 21.1°, 21.8°, 22.7°, 23.7°, 27.3°, 27.6°, and 29.7° 2θ, each of which is ±0.2° 2θ.

In some embodiments, Compound 1 Hydrochloride Form HA has an XRPD pattern comprising degree 2θ-reflections at least at one degree 2θ-reflection identified in Table 3C. In some embodiments, Compound 1 Hydrochloride Form HA has an XRPD pattern comprising degree 2θ-reflections at least at two degree 2θ-reflections identified in Table 3C. In some embodiments, Compound 1 Hydrochloride Form HA has an XRPD pattern comprising degree 2θ-reflections at least at three degree 2θ-reflections identified in Table 3C. In some embodiments, Compound 1 Hydrochloride Form HA has an XRPD pattern comprising degree 2θ-reflections at least at four degree 2θ-reflections identified in Table 3C. In some embodiments, Compound 1 Hydrochloride Form HA has an XRPD pattern comprising degree 2θ-reflections at least at five degree 2θ-reflections identified in Table 3C. In some embodiments, Compound 1 Hydrochloride Form HA has an XRPD pattern comprising degree 2θ-reflections at least at six degree 2θ-reflections identified in Table 3C. In some embodiments, Compound 1 Hydrochloride Form HA has an XRPD pattern comprising degree 2θ-reflections at least at seven degree 2θ-reflections identified in Table 3C. In some embodiments, Compound 1 Hydrochloride Form HA has an XRPD pattern comprising degree 2θ-reflections at least at eight degree 2θ-reflections identified in Table 3C.

In some embodiments, Compound 1 Hydrochloride Form HA has an XRPD pattern comprising degree 2θ-reflections at least at one peak identified in Table 3C. In some embodiments, Compound 1 Hydrochloride Form HA has an XRPD pattern comprising degree 2θ-reflections at least at three peaks identified in Table 3C. In some embodiments, Compound 1 Hydrochloride Form HA has an XRPD pattern comprising degree 2θ-reflections at least at four peaks identified in Table 3C. In some embodiments, Compound 1 Hydrochloride Form HA has an XRPD pattern comprising degree 2θ-reflections at least at five peaks identified in Table 3C. In some embodiments, Compound 1 Hydrochloride Form HA has an XRPD pattern comprising degree 2θ-reflections at least at six peaks identified in Table 3C. In some embodiments, Compound 1 Hydrochloride Form HA has an XRPD pattern comprising degree 2θ-reflections at least at seven peaks identified in Table 3C. In some embodiments, Compound 1 Hydrochloride Form HA has an XRPD pattern comprising degree 2θ-reflections at least at eight peaks identified in Table 3C.

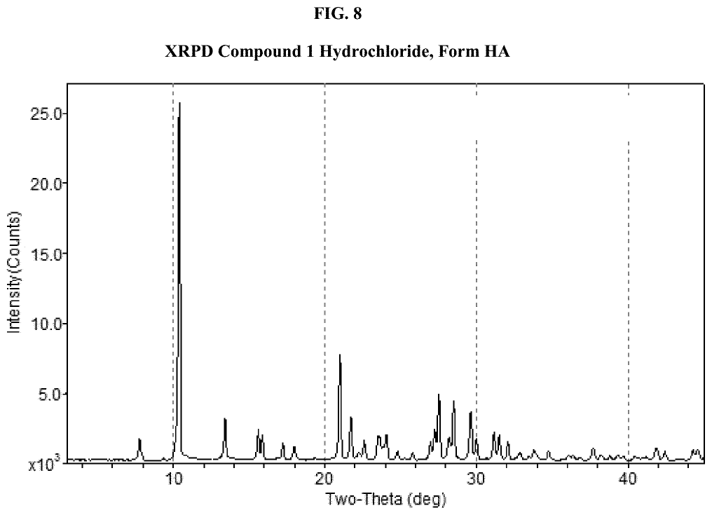

In some embodiments, Compound 1 Hydrochloride Form HA has an XRPD pattern with characteristic peaks as substantially shown in FIG. 8.

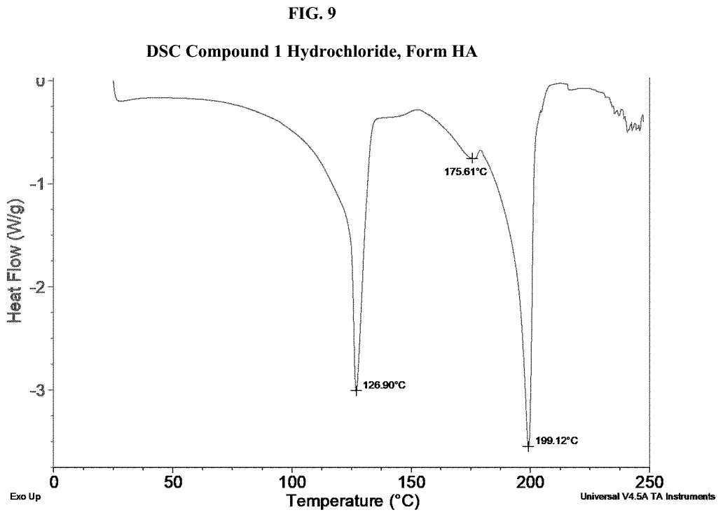

In some embodiments, Compound 1 Hydrochloride Form HA has an endotherm peak at temperature of about 127° C. In some embodiments, Compound 1 Hydrochloride Form HA has an endotherm peak at temperature of about 199° C. In some embodiments, Form HA has endotherm peaks at temperatures of about 127° C. and about 199° C. In some embodiments, Compound 1 Hydrochloride Form HA has a DSC thermogram substantially as depicted in FIG. 9.

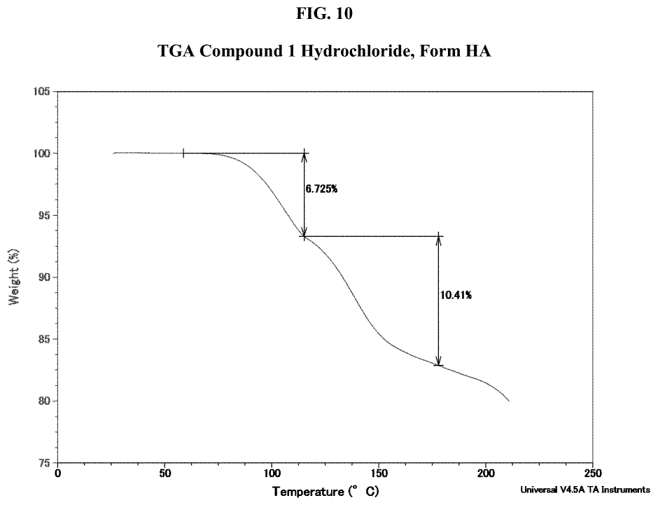

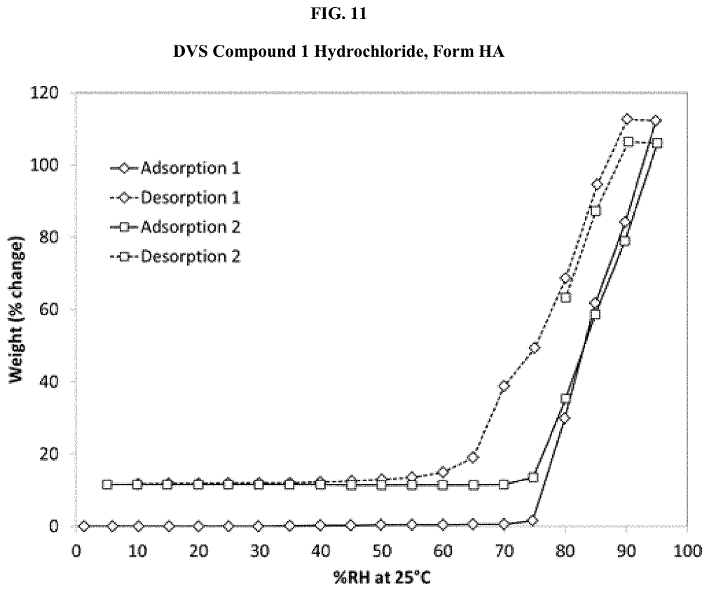

In some embodiments, Compound 1 Hydrochloride Form HA has a DVS isotherm substantially as depicted in FIG. 11. In some embodiments, Compound 1 Hydrochloride Form HA has a TGA thermogram substantially as depicted in FIG. 10.

In some embodiments, Compound 1 Hydrochloride Form HA has at least one characteristic XRPD peak selected from about 7.8, about 10.2, and about 13.4 degrees 2-theta; and an endotherm peak at temperature of about 127° C. In some embodiments, Compound 1 Hydrochloride Form HA has at least one characteristic XRPD peak selected from about 7.8, about 10.2, and about 13.4 degrees 2-theta; and an endotherm peak at temperature of about 199° C. In some embodiments, Compound 1 Hydrochloride Form HA has at least one characteristic XRPD peak selected from about 7.8, about 10.2, and about 13.4 degrees 2-theta; endotherm peaks at temperatures of about 127° C. and about 199° C. In some embodiments, Compound 1 Hydrochloride Form HA has at least one characteristic XRPD peak selected from about 7.8, about 10.2, and about 13.4 degrees 2-theta; and a DSC thermogram substantially as depicted in FIG. 9.

In some embodiments, Compound 1 Hydrochloride Form HA has at least one characteristic XRPD peak selected from about 7.8, about 10.2, and about 13.4 degrees 2-theta; and a DVS isotherm substantially as depicted in FIG. 11. In some embodiments, Compound 1 Hydrochloride Form HA has at least one characteristic XRPD peak selected from about 7.8, about 10.2, and about 13.4 degrees 2-theta; and a TGA thermogram substantially as depicted in FIG. 10.

In some embodiments, Form HA can be isolated with a purity of at least about 80%, about 85%, about 90%, about 95%, about 96%, about 97%, about 98%, or about 99%. In some embodiments, Compound 1 Hydrochloride Form HA can be isolated with a purity greater than about 99%. In some embodiments, Compound 1 Hydrochloride Form HA can be isolated with a purity greater than about 99.9%.

In some embodiments, provided is crystalline (R)-1-(3,4-dihydro-1H-[1,4]oxazino[4,3-b]indazol-1-yl)-N-methylmethanamine hydrochloride Form HB (Compound 1 Hydrochloride Form HB).

In some embodiments, provided is crystalline (R)-1-(3,4-dihydro-1H-[1,4]oxazino[4,3-b]indazol-1-yl)-N-methylmethanamine hydrochloride Form HC (Compound 1 Hydrochloride Form HC). In some embodiments, Compound 1 Hydrochloride Form HC is a mono-hydrochloride. In some embodiments, Compound 1 Hydrochloride Form HC is a mono-hydrate.

In some embodiments, Compound 1 Hydrochloride Form HC has at least one characteristic XRPD peak selected from about 8.4, about 10.4, and about 11.0 degrees 2-theta. In some embodiments, Compound 1 Hydrochloride Form HC has at least one characteristic XRPD peak selected from about 8.4, about 10.4, about 11.0, about 14.0, about 16.4, and about 16.9 degrees 2-theta.

In some embodiments, Form Compound 1 Hydrochloride HC has at least two characteristic XRPD peaks selected from about 8.4, about 10.4, about 11.0, about 14.0, about 16.4, about 16.9, about 18.3, about 22.1, about 26.4, about 27.1, and about 29.1 degrees 2-theta.

In some embodiments, Compound 1 Hydrochloride Form HC has at least three characteristic XRPD peaks selected from about 8.4, about 10.4, about 11.0, about 14.0, about 16.4, about 16.9, about 18.3, about 22.1, about 26.4, about 27.1, and about 29.1 degrees 2-theta.

In some embodiments, Compound 1 Hydrochloride Form HC has an XRPD pattern comprising degree 2θ-reflections at 8.4°, 10.4°, and 11.0° 2θ, each of which is ±0.2° 2θ. In some embodiments, Compound 1 Hydrochloride Form HC has an XRPD pattern comprising degree 2θ-reflections at 8.4°, 10.4°, 11.0°, 14.0°, 16.4°, and 16.9° 2θ, each of which is ±0.2° 2θ. In some embodiments, Compound 1 Hydrochloride Form HC has an XRPD pattern comprising degree 2θ-reflections at 8.4°, 10.4°, 11.0°, 14.0°, 16.4°, 16.9°, 18.3°, 22.1°, 26.4°, 27.1°, and 29.1° 2θ, each of which is ±0.2° 2θ.

In some embodiments, Compound 1 Hydrochloride Form HC has an XRPD pattern comprising degree 2θ-reflections at 8.4°, 10.4°, and 11.0° 2θ, each of which is ±0.2° 2θ, and at least one degree 2θ-reflection selected from 14.0°, 16.4°, 16.9°, 18.3°, 22.1°, 26.4°, 27.1°, and 29.1° 2θ, each of which is ±0.2° 2θ. In some embodiments, Compound 1 Hydrochloride Form HC has an XRPD pattern comprising degree 2θ-reflections at 8.4°, 10.4°, and 11.0° 2θ, each of which is ±0.2° 2θ, and at least two degree 2θ-reflections selected from 14.0°, 16.4°, 16.9°, 18.3°, 22.1°, 26.4°, 27.1°, and 29.1° 2θ, each of which is ±0.2° 2θ. In some embodiments, Compound 1 Hydrochloride Form HC has an XRPD pattern comprising degree 2θ-reflections at 8.4°, 10.4°, and 11.0° 2θ, each of which is ±0.2° 2θ, and at least three degree 2θ-reflections selected from 14.0°, 16.4°, 16.9°, 18.3°, 22.1°, 26.4°, 27.1°, and 29.1° 2θ, each of which is ±0.2° 2θ. In some embodiments, Compound 1 Hydrochloride Form HC has an XRPD pattern comprising degree 2θ-reflections at 8.4°, 10.4°, and 11.0° 2θ, each of which is ±0.2° 2θ, and at least four degree 2θ-reflections selected from 14.0°, 16.4°, 16.9°, 18.3°, 22.1°, 26.4°, 27.1°, and 29.1° 2θ, each of which is ±0.2° 2θ.

In some embodiments, Compound 1 Hydrochloride Form HC has an XRPD pattern comprising degree 2θ-reflections at least at one degree 2θ-reflection identified in Table 3D. In some embodiments, Compound 1 Hydrochloride Form HC has an XRPD pattern comprising degree 2θ-reflections at least at two degree 2θ-reflections identified in Table 3D. In some embodiments, Compound 1 Hydrochloride Form HC has an XRPD pattern comprising degree 2θ-reflections at least at three degree 2θ-reflections identified in Table 3D. In some embodiments, Compound 1 Hydrochloride Form HC has an XRPD pattern comprising degree 2θ-reflections at least at four degree 2θ-reflections identified in Table 3D. In some embodiments, Compound 1 Hydrochloride Form HC has an XRPD pattern comprising degree 2θ-reflections at least at five degree 2θ-reflections identified in Table 3D. In some embodiments, Compound 1 Hydrochloride Form HC has an XRPD pattern comprising degree 2θ-reflections at least at six degree 2θ-reflections identified in Table 3D. In some embodiments, Compound 1 Hydrochloride Form HC has an XRPD pattern comprising degree 2θ-reflections at least at seven degree 2θ-reflections identified in Table 3D. In some embodiments, Compound 1 Hydrochloride Form HC has an XRPD pattern comprising degree 2θ-reflections at least at eight degree 2θ-reflections identified in Table 3D.

In some embodiments, Compound 1 Hydrochloride Form HC has an XRPD pattern comprising degree 2θ-reflections at least at one peak identified in Table 3D. In some embodiments, Compound 1 Hydrochloride Form HC has an XRPD pattern comprising degree 2θ-reflections at least at two peaks identified in Table 3D. In some embodiments, Compound 1 Hydrochloride Form HC has an XRPD pattern comprising degree 2θ-reflections at least at three peaks identified in Table 3D. In some embodiments, Compound 1 Hydrochloride Form HC has an XRPD pattern comprising degree 2θ-reflections at least at four peaks identified in Table 3D. In some embodiments, Compound 1 Hydrochloride Form HC has an XRPD pattern comprising degree 2θ-reflections at least at five peaks identified in Table 3D. In some embodiments, Compound 1 Hydrochloride Form HC has an XRPD pattern comprising degree 2θ-reflections at least at six peaks identified in Table 3D. In some embodiments, Compound 1 Hydrochloride Form HC has an XRPD pattern comprising degree 2θ-reflections at least at seven peaks identified in Table 3D. In some embodiments, Compound 1 Hydrochloride Form HC has an XRPD pattern comprising degree 2θ-reflections at least at eight peaks identified in Table 3D.

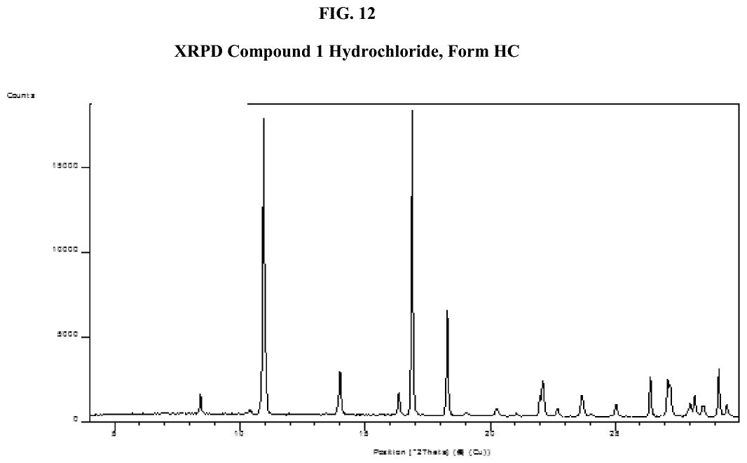

In some embodiments, Compound 1 Hydrochloride Form HC has an XRPD pattern with characteristic peaks as substantially shown in FIG. 12.

In some embodiments, Compound 1 Hydrochloride Form HC has an endotherm peak at temperature of about 131° C. In some embodiments, Compound 1 Hydrochloride Form HC has an endotherm peak at temperature of about 216° C. In some embodiments, Compound 1 Hydrochloride Form HC an exotherm peak at temperature of about 182° C. In some embodiments, Compound 1 Hydrochloride Form HC has endotherm peaks at temperatures of about 131° C. and about 216° C., and an exotherm peak at temperature of about 182° C.

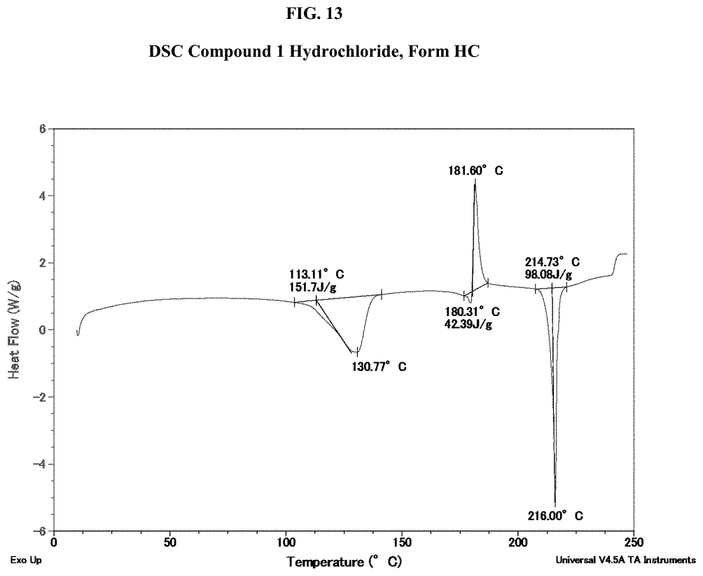

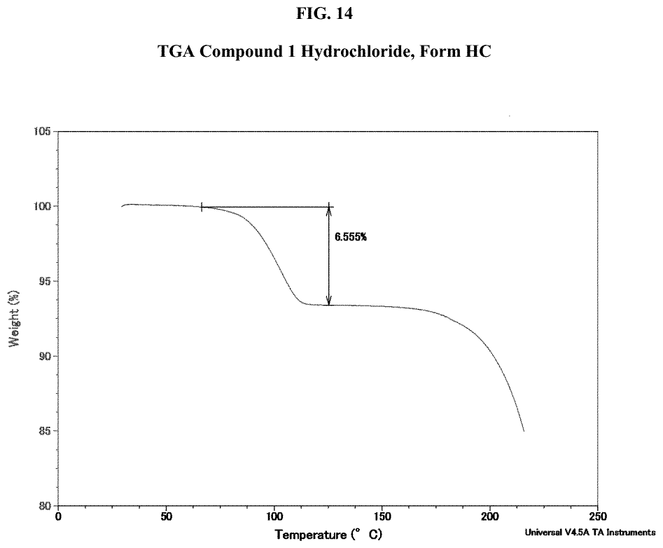

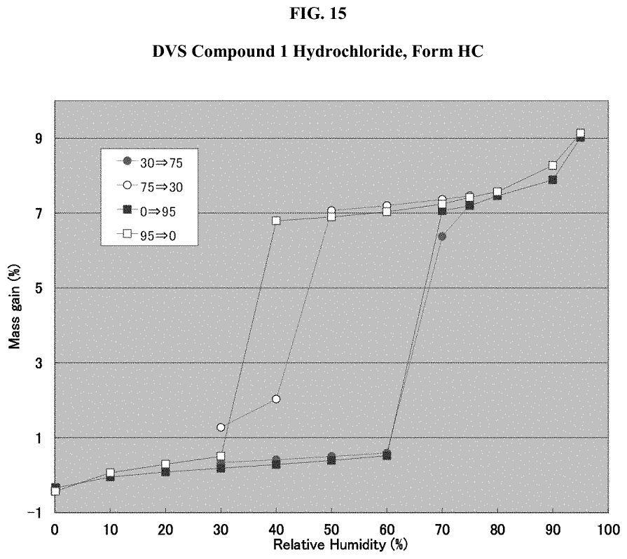

In some embodiments, Compound 1 Hydrochloride Form HC has a DSC thermogram substantially as depicted in FIG. 13. In some embodiments, Compound 1 Hydrochloride Form HC has a DVS isotherm substantially as depicted in FIG. 15. In some embodiments, Compound 1 Hydrochloride Form HC has a TGA thermogram substantially as depicted in FIG. 14.

In some embodiments, Compound 1 Hydrochloride Form HC has at least one characteristic XRPD peak selected from about 8.4, about 10.4, and about 11.0 degrees 2-theta; and an endotherm peak at temperature of about 131° C. In some embodiments, Compound 1 Hydrochloride Form HC has at least one characteristic XRPD peak selected from about 8.4, about 10.4, and about 11.0 degrees 2-theta; and an endotherm peak at temperature of about 216° C. In some embodiments, Compound 1 Hydrochloride Form HC has at least one characteristic XRPD peak selected from about 8.4, about 10.4, and about 11.0 degrees 2-theta; and an exotherm peak at temperature of about 182° C. In some embodiments, Compound 1 Hydrochloride Form HC has at least one characteristic XRPD peak selected from about 8.4, about 10.4, and about 11.0 degrees 2-theta; and endotherm peaks at temperatures of about 131° C. and about 216° C.; and an exotherm peak at temperature of about 182° C.

In some embodiments, Compound 1 Hydrochloride Form HC has at least one characteristic XRPD peak selected from about 8.4, about 10.4, and about 11.0 degrees 2-theta; and a DSC thermogram substantially as depicted in FIG. 13. In some embodiments, Compound 1 Hydrochloride Form HC has at least one characteristic XRPD peak selected from about 8.4, about 10.4, and about 11.0 degrees 2-theta; and a DVS isotherm substantially as depicted in FIG. 15. In some embodiments, Compound 1 Hydrochloride Form HC has at least one characteristic XRPD peak selected from about 8.4, about 10.4, and about 11.0 degrees 2-theta; and a TGA thermogram substantially as depicted in FIG. 14.

In some embodiments, Compound 1 Hydrochloride Form HC can be isolated with a purity of at least about 80%, about 85%, about 90%, about 95%, about 96%, about 97%, about 98%, or about 99%. In some embodiments, Compound 1 Hydrochloride Form HC can be isolated with a purity greater than about 99%. In some embodiments, Compound 1 Hydrochloride Form HC can be isolated with a purity greater than about 99.9%.

In some embodiments, provided is crystalline (R)-1-(3,4-dihydro-1H-[1,4]oxazino[4,3-b]indazol-1-yl)-N-methylmethanamine hydrochloride Form HD (Compound 1 Hydrochloride Form HD). In some embodiments, Compound 1 Hydrochloride Form HD is a mono-hydrochloride. In some embodiments, Compound 1 Hydrochloride Form HD is a di-hydrate.

Provided herein is also a process for preparing crystalline Compound 1 Hydrochloride, comprising precipitating Compound 1 Hydrochloride from a solution comprising Compound 1, hydrochloride acid, and S4, wherein S4 is a solvent. In some embodiments, S4 comprises water, methanol, ethanol, isopropanol, chloroform, acetonitrile, 1,2-dimethoxyethane, tetrahydrofuran, 1,4-dioxane, tbutyl methyl ether, diisopropyl ether, cyclopentyl methyl ether, ethyl acetate, isopropyl acetate, isobutyl acetate, acetone, methyl ethyl ketone, methyl isobutyl ketone, heptane, chlorobenzene, toluene, xylene, or a mixture thereof. For example, the mixture can be methanol and diisopropyl ether; methanol and water; ethanol and water; isopropanol and water; acetonitrile and water; and acetone and water.

In some embodiments, provided is Compound 1 Hydrochloride Form HA prepared by isolating Compound 1 Hydrochloride Form HA from a mixture of Compound 1, hydrochloric acid, and S4, wherein S4 is a solvent. In some embodiments, S4 comprises water, methanol, ethanol, isopropanol, chloroform, acetonitrile, 1,2-dimethoxyethane, tetrahydrofuran, 1,4-dioxane, tbutyl methyl ether, diisopropyl ether, cyclopentyl methyl ether, ethyl acetate, isopropyl acetate, isobutyl acetate, acetone, methyl ethyl ketone, methyl isobutyl ketone, heptane, chlorobenzene, toluene, xylene, or a mixture thereof or a mixture thereof. For example, the mixture can be methanol and diisopropyl ether; methanol and water; ethanol and water; acetonitrile and water; and acetone and water.

In some embodiments, provided is Compound 1 Hydrochloride Form HB prepared by isolating Compound 1 Hydrochloride Form HB from a mixture of Compound 1, hydrochloric acid, and S4, wherein S4 is a solvent. In some embodiments, S4 comprises isopropanol, acetonitrile, acetone, or a mixture thereof. In some embodiments, S4 is a mixture of methanol and diisopropyl ether.

In some embodiments, provided is Compound 1 Hydrochloride Form HC prepared by isolating Compound 1 Hydrochloride Form HC from a mixture of Compound 1, hydrochloric acid, and S4, wherein S4 is a solvent. In some embodiments, S4 comprises isopropanol. In some embodiments, S4 is a mixture of ethanol and water, or isopropanol and water.

In some embodiments, Compound 1 Hydrochloride Form HC can be prepared from Compound 1 Hydrochloride Form HA. For example, Compound 1 Hydrochloride Form HC can be precipitated from a solution comprising Compound Hydrochloride Form HA, ethanol, and water (e.g., 10:1 of ethanol to water).

In some embodiments, provided is Compound 1 Hydrochloride Form HD prepared by isolating Compound 1 Hydrochloride Form HD from a mixture of Compound 1, hydrochloric acid, and S4, wherein S4 is a solvent. In some embodiments, S4 is a mixture of isopropanol and water.

### Compound 1 Fumarate

Provided herein is a fumaric acid salt of Compound 1: (R)-1-(3,4-dihydro-1H-[1,4]oxazino[4,3-b]indazol-1-yl)-N-methylmethanamine fumarate, which is described herein, including the Examples. In some embodiments, provided is anhydrous (R)-1-(3,4-dihydro-1H-[1,4]oxazino[4,3-b]indazol-1-yl)-N-methylmethanamine fumarate (anhydrous Compound 1 Fumarate). In some embodiments, provided is crystalline (R)-1-(3,4-dihydro-1H-[1,4]oxazino[4,3-b]indazol-1-yl)-N-methylmethanamine fumarate (crystalline Compound 1 Fumarate). In some embodiments, provided is a hydrate of (R)-1-(3,4-dihydro-1H-[1,4]oxazino[4,3-b]indazol-1-yl)-N-methylmethanamine fumarate (Compound 1 Fumarate Hydrate).

In some embodiments, provided is crystalline (R)-1-(3,4-dihydro-1H-[1,4]oxazino[4,3-b]indazol-1-yl)-N-methylmethanamine fumarate Form FA (Compound 1 Fumarate Form FA). In some embodiments, provided is Compound 1 Fumarate Form FA di-hydrate.

In some embodiments, Compound 1 Fumarate Form FA has at least one characteristic XRPD peak selected from about 9.9, about 11.4, and about 12.0 degrees 2-theta. In some embodiments, Compound 1 Fumarate Form FA has at least one characteristic XRPD peak selected from about 9.9, about 11.4, about 12.0, about 14.8, about 15.5, about 15.8, about 16.3, and about 17.2 degrees 2-theta.

In some embodiments, Compound 1 Fumarate Form FA has at least two characteristic XRPD peaks selected from about 9.9, about 11.4, about 12.0, about 14.8, about 15.5, about 15.8, about 16.3, about 17.2, about 18.7, about 19.3, about 19.8, about 21.7, about 24.0, about 25.7, and about 27.7 degrees 2-theta.

In some embodiments, Compound 1 Fumarate Form FA has at least three characteristic XRPD peaks selected from about 9.9, about 11.4, about 12.0, about 14.8, about 15.5, about 15.8, about 16.3, about 17.2, about 18.7, about 19.3, about 19.8, about 21.7, about 24.0, about 25.7, and about 27.7 degrees 2-theta.

In some embodiments, Compound 1 Fumarate Form FA has an XRPD pattern comprising degree 2θ-reflections at 9.9°, 11.4°, and 12.0° 2θ, each of which is ±0.2° 2θ. In some embodiments, Compound 1 Fumarate Form FA has an XRPD pattern comprising degree 2θ-reflections at 9.9°, 11.4°, 12.0°, 14.8°, 15.5°, 15.8°, 16.3°, and 17.2° 2θ, each of which is ±0.2° 2θ. In some embodiments, Compound 1 Fumarate Form FA has an XRPD pattern comprising degree 2θ-reflections at 9.9°, 11.4°, 12.0°, 14.8°, 15.5°, 15.8°, 16.3°, 17.2°, 18.7°, 19.3°, 19.8°, 21.7°, 24.0°, 25.7°, and 27.7° 2θ, each of which is ±0.2° 2θ.

In some embodiments, Compound 1 Fumarate Form FA has an XRPD pattern comprising degree 2θ-reflections at 9.9°, 11.4°, and 12.0° 2θ, each of which is ±0.2° 2θ, and at least one degree 2θ-reflection selected from 14.8°, 15.5°, 15.8°, 16.3°, 17.2°, 18.7°, 19.3°, 19.8°, 21.7°, 24.0°, 25.7°, and 27.7° 2θ, each of which is ±0.2° 2θ. In some embodiments, Compound 1 Fumarate Form FA has an XRPD pattern comprising degree 2θ-reflections at 9.9°, 11.4°, and 12.0° 2θ, each of which is ±0.2° 2θ, and at least two degree 2θ-reflections selected from 14.8°, 15.5°, 15.8°, 16.3°, 17.2°, 18.7°, 19.3°, 19.8°, 21.7°, 24.0°, 25.7°, and 27.7° 2θ, each of which is ±0.2° 2θ. In some embodiments, Compound 1 Fumarate Form FA has an XRPD pattern comprising degree 2θ-reflections at 9.9°, 11.4°, and 12.0° 2θ, each of which is ±0.2° 2θ, and at least three degree 2θ-reflections selected from 14.8°, 15.5°, 15.8°, 16.3°, 17.2°, 18.7°, 19.3°, 19.8°, 21.7°, 24.0°, 25.7°, and 27.7° 2θ, each of which is ±0.2° 2θ. In some embodiments, Compound 1 Fumarate Form FA has an XRPD pattern comprising degree 2θ-reflections at 9.9°, 11.4°, and 12.0° 2θ, each of which is ±0.2° 2θ, and at least four degree 2θ-reflections selected from 14.8°, 15.5°, 15.8°, 16.3°, 17.2°, 18.7°, 19.3°, 19.8°, 21.7°, 24.0°, 25.7°, and 27.7° 2θ, each of which is ±0.2° 2θ.

In some embodiments, Compound 1 Fumarate Form FA has an XRPD pattern comprising degree 2θ-reflections at least at one degree 2θ-reflection identified in Table 6A. In some embodiments, Compound 1 Fumarate Form FA has an XRPD pattern comprising degree 2θ-reflections at least at two degree 2θ-reflections identified in Table 6A. In some embodiments, Compound 1 Fumarate Form FA has an XRPD pattern comprising degree 2θ-reflections at least at three degree 2θ-reflections identified in Table 6A. In some embodiments, Compound 1 Fumarate Form FA has an XRPD pattern comprising degree 2θ-reflections at least at four degree 2θ-reflections identified in Table 6A. In some embodiments, Compound 1 Fumarate Form FA has an XRPD pattern comprising degree 2θ-reflections at least at five degree 2θ-reflections identified in Table 6A. In some embodiments, Compound 1 Fumarate Form FA has an XRPD pattern comprising degree 2θ-reflections at least at six degree 2θ-reflections identified in Table 6A. In some embodiments, Compound 1 Fumarate Form FA has an XRPD pattern comprising degree 2θ-reflections at least at seven degree 2θ-reflections identified in Table 6A. In some embodiments, Compound 1 Fumarate Form FA has an XRPD pattern comprising degree 2θ-reflections at least at eight degree 2θ-reflections identified in Table 6A.

In some embodiments, Compound 1 Fumarate Form FA has an XRPD pattern comprising degree 2θ-reflections at least at one peak identified in Table 6A. In some embodiments, Compound 1 Fumarate Form FA has an XRPD pattern comprising degree 2θ-reflections at least at two peaks identified in Table 6A. In some embodiments, Compound 1 Fumarate Form FA has an XRPD pattern comprising degree 2θ-reflections at least at three peaks identified in Table 6A. In some embodiments, Compound 1 Fumarate Form FA has an XRPD pattern comprising degree 2θ-reflections at least at four peaks identified in Table 6A. In some embodiments, Compound 1 Fumarate Form FA has an XRPD pattern comprising degree 2θ-reflections at least at five peaks identified in Table 6A. In some embodiments, Compound 1 Fumarate Form FA has an XRPD pattern comprising degree 2θ-reflections at least at six peaks identified in Table 6A. In some embodiments, Compound 1 Fumarate Form FA has an XRPD pattern comprising degree 2θ-reflections at least at seven peaks identified in Table 6A. In some embodiments, Compound 1 Fumarate Form FA has an XRPD pattern comprising degree 2θ-reflections at least at eight peaks identified in Table 6A.

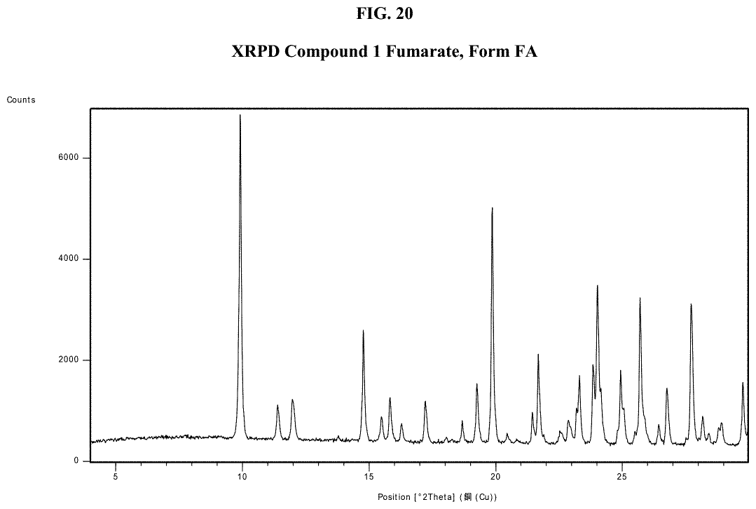

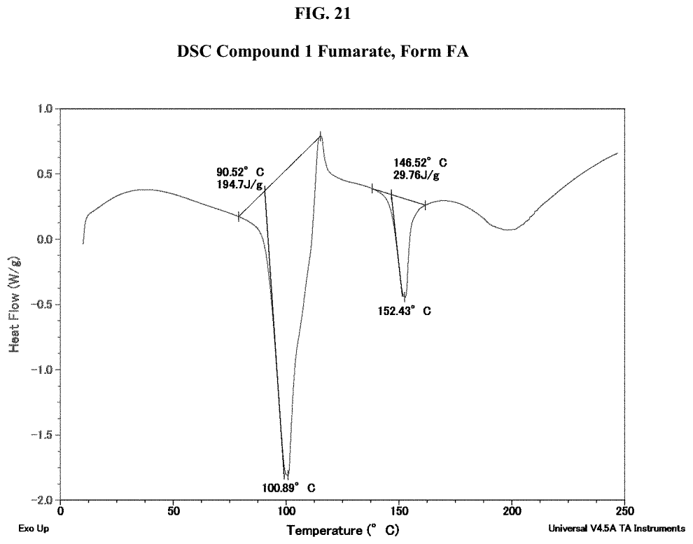

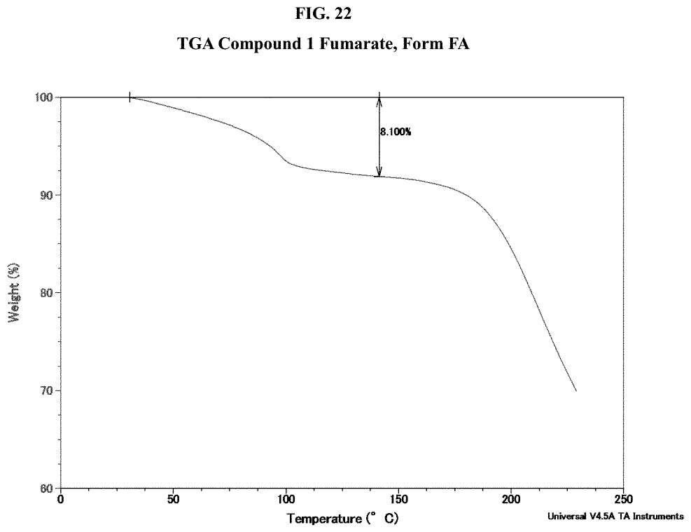

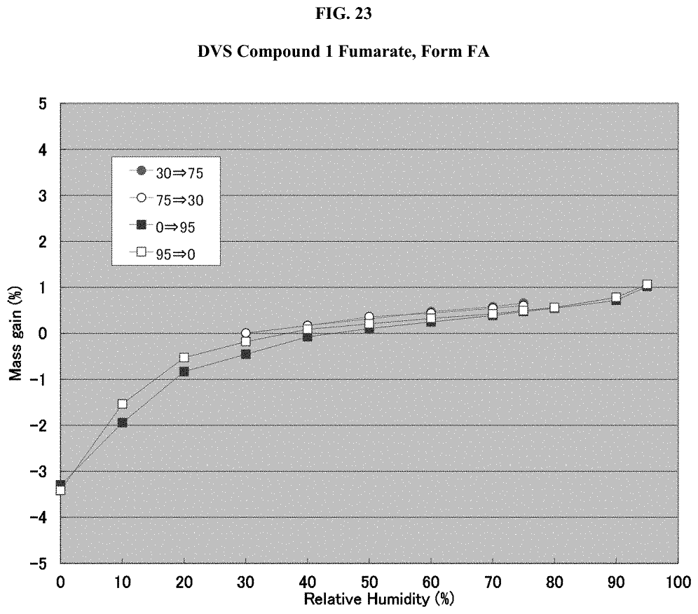

In some embodiments, Compound 1 Fumarate Form FA has an XRPD pattern with characteristic peaks as substantially shown in FIG. 20. In some embodiments, Compound 1 Fumarate Form FA has endotherm peaks at temperatures of about 101° C. and about 152° C. In some embodiments, Compound 1 Fumarate Form FA has a DSC thermogram substantially as depicted in FIG. 21. In some embodiments, Compound 1 Fumarate Form BA has a DVS isotherm substantially as depicted in FIG. 23. In some embodiments, Compound 1 Fumarate Form FA has a TGA thermogram substantially as depicted in FIG. 22.

In some embodiments, Compound 1 Fumarate Form FA has at least one characteristic XRPD peak selected from about 9.9, about 11.4, and about 12.0 degrees 2-theta; and endotherm peaks at temperatures of about 101° C. and about 152° C. In some embodiments, Compound 1 Fumarate Form FA has at least one characteristic XRPD peak selected from about 9.9, about 11.4, and about 12.0 degrees 2-theta; and a DSC thermogram substantially as depicted in FIG. 21. In some embodiments, Compound 1 Fumarate Form FA has at least one characteristic XRPD peak selected from about 9.9, about 11.4, and about 12.0 degrees 2-theta; and a DVS isotherm substantially as depicted in FIG. 23. In some embodiments, Compound 1 Fumarate Form FA has at least one characteristic XRPD peak selected from about 9.9, about 11.4, and about 12.0 degrees 2-theta; and a TGA thermogram substantially as depicted in FIG. 22.

In some embodiments, Compound 1 Fumarate Form FA can be isolated with a purity of at least about 80%, about 85%, about 90%, about 95%, about 96%, about 97%, about 98%, or about 99%. In some embodiments, Compound 1 Fumarate Form FA can be isolated with a purity greater than about 99%. In some embodiments, Compound 1 Fumarate Form FA can be isolated with a purity greater than about 99.9%.

In some embodiments, provided is crystalline (R)-1-(3,4-dihydro-1H-[1,4]oxazino[4,3-b]indazol-1-yl)-N-methylmethanamine fumarate Form FB (Compound 1 Fumarate Form FB). In some embodiments, provided is Compound 1 Fumarate Form FB mono-hydrate.

In some embodiments, Compound 1 Fumarate Form FB has at least one characteristic XRPD peak selected from about 8.8, about 9.1, about 14.6, and about 14.8 degrees 2-theta. In some embodiments, Compound 1 Fumarate Form FB has at least one characteristic XRPD peak selected from about 8.8, about 9.1, about 11.9, about 12.4, about 14.6, about 14.8, about 16.0, about 16.6, and about 17.7 degrees 2-theta.

In some embodiments, Compound 1 Fumarate Form FB has at least two characteristic XRPD peaks selected from about 8.8, about 9.1, about 11.9, about 12.4, about 14.6, about 14.8, about 16.0, about 16.6, about 17.7, about 19.4, about 20.3, about 20.8, about 21.7, about 23.7, about 23.9, and about 24.6 degrees 2-theta.

In some embodiments, Compound 1 Fumarate Form FB has at least three characteristic XRPD peaks selected from about 8.8, about 9.1, about 11.9, about 12.4, about 14.6, about 14.8, about 16.0, about 16.6, about 17.7, about 19.4, about 20.3, about 20.8, about 21.7, about 23.7, about 23.9, and about 24.6 degrees 2-theta.

In some embodiments, Compound 1 Fumarate Form FB has an XRPD pattern comprising degree 2θ-reflections at 8.8°, 9.1°, 14.6°, and 14.8° 2θ, each of which is ±0.2° 2θ. In some embodiments, Compound 1 Fumarate Form FB has an XRPD pattern comprising degree 2θ-reflections at 8.8°, 9.1°, 11.9°, 12.4°, 14.6°, 14.8°, 16.0°, 16.6°, and 17.7° 2θ, each of which is ±0.2° 2θ. In some embodiments, Compound 1 Fumarate Form FB has an XRPD pattern comprising degree 2θ-reflections at 8.8°, 9.1°, 11.9°, 12.4°, 14.6°, 14.8°, 16.0°, 16.6°, 17.7°, 19.4°, 20.3°, 20.8°, 21.7°, 23.7°, 23.9°, and 24.6° 2θ, each of which is ±0.2° 2θ.

In some embodiments, Compound 1 Fumarate Form FB has an XRPD pattern comprising degree 2θ-reflections at 8.8°, 9.1°, 14.6°, and 14.8° 2θ, each of which is ±0.2° 2θ, and at least one degree 2θ-reflection selected from 11.9°, 12.4°, 16.0°, 16.6°, 17.7°, 19.4°, 20.3°, 20.8°, 21.7°, 23.7°, 23.9°, and 24.6° 2θ, each of which is ±0.2° 2θ. In some embodiments, Compound 1 Fumarate Form FB has an XRPD pattern comprising degree 2θ-reflections at 8.8°, 9.1°, 14.6°, and 14.8° 2θ, each of which is ±0.2° 2θ, and at least two degree 2θ-reflections selected from 11.9°, 12.4°, 16.0°, 16.6°, 17.7°, 19.4°, 20.3°, 20.8°, 21.7°, 23.7°, 23.9°, and 24.6° 2θ, each of which is ±0.2° 2θ. In some embodiments, Compound 1 Fumarate Form FB has an XRPD pattern comprising degree 2θ-reflections at 8.8°, 9.1°, 14.6°, and 14.8° 2θ, each of which is ±0.2° 2θ, and at least three degree 2θ-reflections selected from 11.9°, 12.4°, 16.0°, 16.6°, 17.7°, 19.4°, 20.3°, 20.8°, 21.7°, 23.7°, 23.9°, and 24.6° 2θ, each of which is ±0.2° 2θ. In some embodiments, Compound 1 Fumarate Form FB has an XRPD pattern comprising degree 2θ-reflections at 8.8°, 9.1°, 14.6°, and 14.8° 2θ, each of which is ±0.2° 2θ, and at least four degree 2θ-reflections selected from 11.9°, 12.4°, 16.0°, 16.6°, 17.7°, 19.4°, 20.3°, 20.8°, 21.7°, 23.7°, 23.9°, and 24.6° 2θ, each of which is ±0.2° 2θ.

In some embodiments, Compound 1 Fumarate Form FB has an XRPD pattern comprising degree 2θ-reflections at least at one degree 2θ-reflection identified in Table 6B. In some embodiments, Compound 1 Fumarate Form FB has an XRPD pattern comprising degree 2θ-reflections at least at two degree 2θ-reflections identified in Table 6B. In some embodiments, Compound 1 Fumarate Form FB has an XRPD pattern comprising degree 2θ-reflections at least at three degree 2θ-reflections identified in Table 6B. In some embodiments, Compound 1 Fumarate Form FB has an XRPD pattern comprising degree 2θ-reflections at least at four degree 2θ-reflections identified in Table 6B. In some embodiments, Compound 1 Fumarate Form FB has an XRPD pattern comprising degree 2θ-reflections at least at five degree 2θ-reflections identified in Table 6B. In some embodiments, Compound 1 Fumarate Form FB has an XRPD pattern comprising degree 2θ-reflections at least at six degree 2θ-reflections identified in Table 6B. In some embodiments, Compound 1 Fumarate Form FB has an XRPD pattern comprising degree 2θ-reflections at least at seven degree 2θ-reflections identified in Table 6B. In some embodiments, Compound 1 Fumarate Form FB has an XRPD pattern comprising degree 2θ-reflections at least at eight degree 2θ-reflections identified in Table 6B.

In some embodiments, Compound 1 Fumarate Form FB has an XRPD pattern comprising degree 2θ-reflections at least at one peak identified in Table 6B. In some embodiments, Compound 1 Fumarate Form FB has an XRPD pattern comprising degree 2θ-reflections at least at two peaks identified in Table 6B. In some embodiments, Compound 1 Fumarate Form FB has an XRPD pattern comprising degree 2θ-reflections at least at three peaks identified in Table 6B. In some embodiments, Compound 1 Fumarate Form FB has an XRPD pattern comprising degree 2θ-reflections at least at four peaks identified in Table 6B. In some embodiments, Compound 1 Fumarate Form FB has an XRPD pattern comprising degree 2θ-reflections at least at five peaks identified in Table 6B. In some embodiments, Compound 1 Fumarate Form FB has an XRPD pattern comprising degree 2θ-reflections at least at six peaks identified in Table 6B. In some embodiments, Compound 1 Fumarate Form FB has an XRPD pattern comprising degree 2θ-reflections at least at seven peaks identified in Table 6B. In some embodiments, Compound 1 Fumarate Form FB has an XRPD pattern comprising degree 2θ-reflections at least at eight peaks identified in Table 6B.

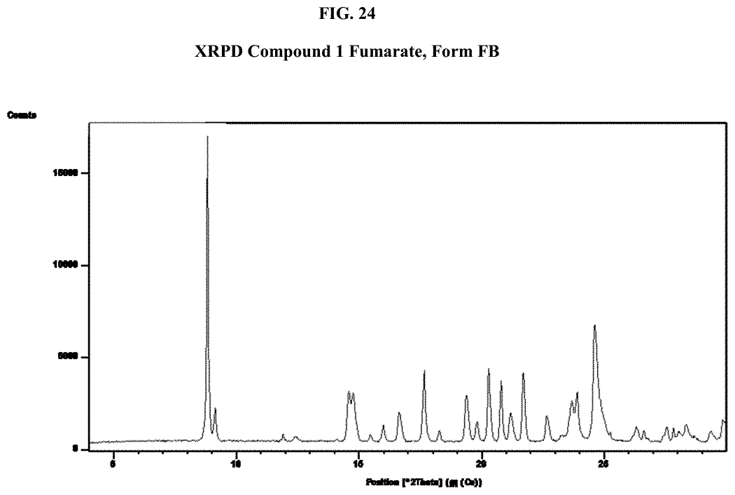

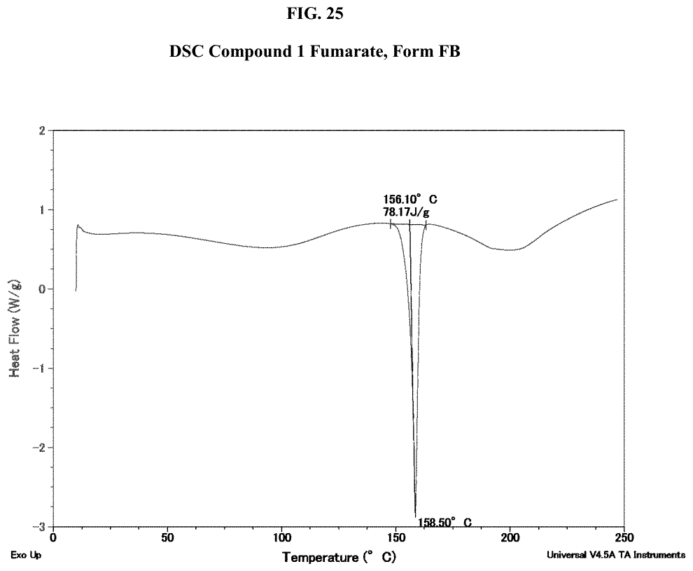

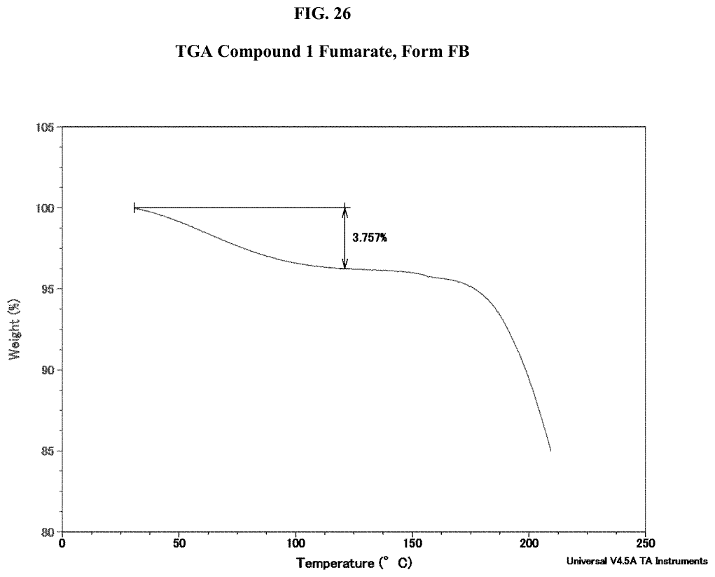

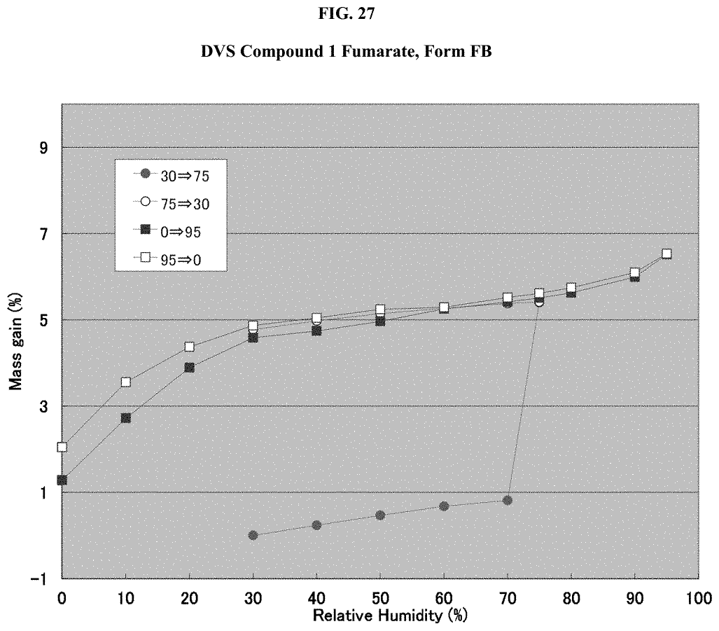

In some embodiments, Compound 1 Fumarate Form FB has an XRPD pattern with characteristic peaks as substantially shown in FIG. 24. In some embodiments, Compound 1 Fumarate Form FB has an endotherm peak at a temperature of about 159° C. In some embodiments, Compound 1 Fumarate Form FB has a DSC thermogram substantially as depicted in FIG. 25. In some embodiments, Compound 1 Fumarate Form FB has a DVS isotherm substantially as depicted in FIG. 27. In some embodiments, Compound 1 Fumarate Form FB has a TGA thermogram substantially as depicted in FIG. 26.

In some embodiments, Compound 1 Fumarate Form FB has at least one characteristic XRPD peak selected from about 8.8, about 9.1, about 14.6, and about 14.8 degrees 2-theta; and an endotherm peak at a temperature of about 159° C. In some embodiments, Compound 1 Fumarate Form FB has at least one characteristic XRPD peak selected from about 8.8, about 9.1, about 14.6, and about 14.8 degrees 2-theta; and a DSC thermogram substantially as depicted in FIG. 25. In some embodiments, Compound 1 Fumarate Form FB has at least one characteristic XRPD peak selected from about 8.8, about 9.1, about 14.6, and about 14.8 degrees 2-theta; and a DVS isotherm substantially as depicted in FIG. 27. In some embodiments, Compound 1 Fumarate Form FB has at least one characteristic XRPD peak selected from about 8.8, about 9.1, about 14.6, and about 14.8 degrees 2-theta; and a TGA thermogram substantially as depicted in FIG. 26.

In some embodiments, Compound 1 Fumarate Form FB can be isolated with a purity of at least about 80%, about 85%, about 90%, about 95%, about 96%, about 97%, about 98%, or about 99%. In some embodiments, Compound 1 Fumarate Form FB can be isolated with a purity greater than about 99%. In some embodiments, Compound 1 Fumarate Form FB can be isolated with a purity greater than about 99.9%.

Provided herein is a process for preparing crystalline Compound 1 Fumarate, comprising precipitating Compound 1 Fumarate from a solution comprising Compound 1, fumaric acid, and S3, wherein S3 is a solvent. In some embodiments, S3 comprises tetrahydrofuran, water, or a mixture thereof.

In some embodiments, provided is Compound 1 Fumarate Form FA prepared by isolating Compound 1 Fumarate Form FA from a mixture of Compound 1, fumaric acid, and S3, wherein S3 is a solvent. In some embodiments, provided is Compound 1 Fumarate Form FA prepared by isolating Compound 1 Fumarate Form FA from a mixture of Compound 1, fumaric acid, and S3, wherein S3 comprises tetrahydrofuran, water, or a mixture thereof.

In some embodiments, provided is Compound 1 Fumarate Form FB prepared by isolating Compound 1 Fumarate Form FB from a mixture of Compound 1, fumaric acid, and S3, wherein S3 is a solvent. In some embodiments, provided is Compound 1 Fumarate Form FB prepared by isolating Compound 1 Fumarate Form FB from a mixture of Compound 1, fumaric acid, and S3, wherein S3 comprises tetrahydrofuran.

In some embodiments, the process to prepare crystalline Compound 1 Fumarate, comprises:
(i)combining fumaric acid and Compound 1;
(ii) adding a solvent (e.g., tetrahydrofuran, water, or a mixture thereof) to the combination of fumaric acid and Compound 1 to provide a mixture;
(iii) shaking the mixture of (ii) in an incubator at about 40° C. to about 80° C. (e.g., about 60° C.) for about 1 to about 4 hours (e.g., about 2 hours);
(iv) storing the mixture of (iii) at room temperature for about 5 to 20 hours to produce crystalline precipitate of Compound 1 Fumarate; and
(v) isolating the crystalline precipitate of (iv).

### Compound 1 Phosphate

Provided herein is a fumaric acid salt of Compound 1: (R)-1-(3,4-dihydro-1H-[1,4]oxazino[4,3-b]indazol-1-yl)-N-methylmethanamine phosphate, which is described herein, including the Examples. In some embodiments, provided is crystalline (R)-1-(3,4-dihydro-1H-[1,4]oxazino[4,3-b]indazol-1-yl)-N-methylmethanamine phosphate (crystalline Compound 1 Phosphate). In some embodiments, provided is crystalline (R)-1-(3,4-dihydro-1H-[1,4]oxazino[4,3-b]indazol-1-yl)-N-methylmethanamine phosphate Form PA (Compound 1 Phosphate Form PA).

In some embodiments, Compound 1 Phosphate Form PA has at least one characteristic XRPD peak selected from about 4.7, about 6.6, about 7.1, and about 8.4 degrees 2-theta. In some embodiments, Compound 1 Phosphate Form PA has at least one characteristic XRPD peak selected from about 4.7, about 6.6, about 7.1, about 8.4, about 9.0, and about 9.4 degrees 2-theta. In some embodiments, Compound 1 Phosphate Form PA has at least one characteristic XRPD peak selected from about 4.7, about 6.6, about 7.1, about 8.4, about 9.0, about 9.4, about 10.0, about 10.3, about 10.6, and about 11.1 degrees 2-theta.

In some embodiments, Compound 1 Phosphate Form PA has at least two characteristic XRPD peaks selected from about 4.7, about 6.6, about 7.1, about 8.4, about 9.0, about 9.4, about 10.0, about 10.3, about 10.6, about 11.1, about 13.2, about 13.6, about 14.2, about 14.5, about 14.6, about 15.4, and about 16.1 degrees 2-theta.

In some embodiments, Compound 1 Phosphate Form PA has at least three characteristic XRPD peaks selected from about 4.7, about 6.6, about 7.1, about 8.4, about 9.0, about 9.4, about 10.0, about 10.3, about 10.6, about 11.1, about 13.2, about 13.6, about 14.2, about 14.5, about 14.6, about 15.4, about 16.1, about 18.3, about 18.8, about 20.3, about 23.6, and about 25.1 degrees 2-theta.

In some embodiments, Compound 1 Phosphate Form PA has an XRPD pattern comprising degree 2θ-reflections at 4.7°, 6.6°, 7.1°, and 8.4° 2θ, each of which is ±0.2° 2θ. In some embodiments, Compound 1 Phosphate Form PA has an XRPD pattern comprising degree 2θ-reflections at 4.7°, 6.6°, 7.1°, 8.4°, 9.0°, and 9.4° 2θ, each of which is ±0.2° 2θ. In some embodiments, Compound 1 Phosphate Form PA has an XRPD pattern comprising degree 2θ-reflections at 4.7°, 6.6°, 7.1°, 8.4°, 9.0°, 9.4°, 10.0°, 10.3°, 10.6°, and 11.1° 2θ, each of which is ±0.2° 2θ. In some embodiments, Compound 1 Phosphate Form PA has an XRPD pattern comprising degree 2θ-reflections at 4.7°, 6.6°, 7.1°, 8.4°, 9.0°, 9.4°, 10.0°, 10.3°, 10.6°, 11.1°, 13.2°, 13.6°, 14.2°, 14.5°, 14.6°, 15.4°, and 16.1° 2θ, each of which is ±0.2° 2θ. In some embodiments, Compound 1 Phosphate Form PA has an XRPD pattern comprising degree 2θ-reflections at 4.7°, 6.6°, 7.1°, 8.4°, 9.0°, 9.4°, 10.0°, 10.3°, 10.6°, 11.1°, 13.2°, 13.6°, 14.2°, 14.5°, 14.6°, 15.4°, 16.1°, 18.3°, 18.8°, 20.3°, 23.6°, and 25.1° 2θ, each of which is ±0.2° 2θ.

In some embodiments, Compound 1 Phosphate Form PA has an XRPD pattern comprising degree 2θ-reflections at 4.7°, 6.6°, 7.1°, and 8.4° 2θ, each of which is ±0.2° 2θ, and at least one degree 2θ-reflection selected from 9.0°, 9.4°, 10.0°, 10.3°, 10.6°, 11.1°, 13.2°, 13.6°, 14.2°, 14.5°, 14.6°, 15.4°, 16.1°, 18.3°, 18.8°, 20.3°, 23.6°, and 25.1° 2θ, each of which is ±0.2° 2θ. In some embodiments, Compound 1 Phosphate Form PA has an XRPD pattern comprising degree 2θ-reflections at 4.7°, 6.6°, 7.1°, and 8.4° 2θ, each of which is ±0.2° 2θ, and at least two degree 2θ-reflections selected from 9.0°, 9.4°, 10.0°, 10.3°, 10.6°, 11.1°, 13.2°, 13.6°, 14.2°, 14.5°, 14.6°, 15.4°, 16.1°, 18.3°, 18.8°, 20.3°, 23.6°, and 25.1° 2θ, each of which is ±0.2° 2θ. In some embodiments, Compound 1 Phosphate Form PA has an XRPD pattern comprising degree 2θ-reflections at 4.7°, 6.6°, 7.1°, and 8.4° 2θ, each of which is ±0.2° 2θ, and at least three degree 2θ-reflections selected from 9.0°, 9.4°, 10.0°, 10.3°, 10.6°, 11.1°, 13.2°, 13.6°, 14.2°, 14.5°, 14.6°, 15.4°, 16.1°, 18.3°, 18.8°, 20.3°, 23.6°, and 25.1° 2θ, each of which is ±0.2° 2θ. In some embodiments, Compound 1 Phosphate Form PA has an XRPD pattern comprising degree 2θ-reflections at 4.7°, 6.6°, 7.1°, and 8.4° 2θ, each of which is ±0.2° 2θ, and at least four degree 2θ-reflections selected from 9.0°, 9.4°, 10.0°, 10.3°, 10.6°, 11.1°, 13.2°, 13.6°, 14.2°, 14.5°, 14.6°, 15.4°, 16.1°, 18.3°, 18.8°, 20.3°, 23.6°, and 25.1° 2θ, each of which is ±0.2° 2θ.

In some embodiments, Compound 1 Phosphate Form PA has an XRPD pattern comprising degree 2θ-reflections at least at one degree 2θ-reflection identified in Table 7. In some embodiments, Compound 1 Phosphate Form PA has an XRPD pattern comprising degree 2θ-reflections at least at two degree 2θ-reflections identified in Table 7. In some embodiments, Compound 1 Phosphate Form PA has an XRPD pattern comprising degree 2θ-reflections at least at three degree 2θ-reflections identified in Table 7. In some embodiments, Compound 1 Phosphate Form PA has an XRPD pattern comprising degree 2θ-reflections at least at four degree 2θ-reflections identified in Table 7. In some embodiments, Compound 1 Phosphate Form PA has an XRPD pattern comprising degree 2θ-reflections at least at five degree 2θ-reflections identified in Table 7. In some embodiments, Compound 1 Phosphate Form PA has an XRPD pattern comprising degree 2θ-reflections at least at six degree 2θ-reflections identified in Table 7. In some embodiments, Compound 1 Phosphate Form PA has an XRPD pattern comprising degree 2θ-reflections at least at seven degree 2θ-reflections identified in Table 7. In some embodiments, Compound 1 Phosphate Form PA has an XRPD pattern comprising degree 2θ-reflections at least at eight degree 2θ-reflections identified in Table 7.

In some embodiments, Compound 1 Phosphate Form PA has an XRPD pattern comprising degree 2θ-reflections at least at one peak identified in Table 7. In some embodiments, Compound 1 Phosphate Form PA has an XRPD pattern comprising degree 2θ-reflections at least at two peaks identified in Table 7. In some embodiments, Compound 1 Phosphate Form PA has an XRPD pattern comprising degree 2θ-reflections at least at three peaks identified in Table 7. In some embodiments, Compound 1 Phosphate Form PA has an XRPD pattern comprising degree 2θ-reflections at least at four peaks identified in Table 7. In some embodiments, Compound 1 Phosphate Form PA has an XRPD pattern comprising degree 2θ-reflections at least at five peaks identified in Table 7. In some embodiments, Compound 1 Phosphate Form PA has an XRPD pattern comprising degree 2θ-reflections at least at six peaks identified in Table 7. In some embodiments, Compound 1 Phosphate Form PA has an XRPD pattern comprising degree 2θ-reflections at least at seven peaks identified in Table 7. In some embodiments, Compound 1 Phosphate Form PA has an XRPD pattern comprising degree 2θ-reflections at least at eight peaks identified in Table 7.

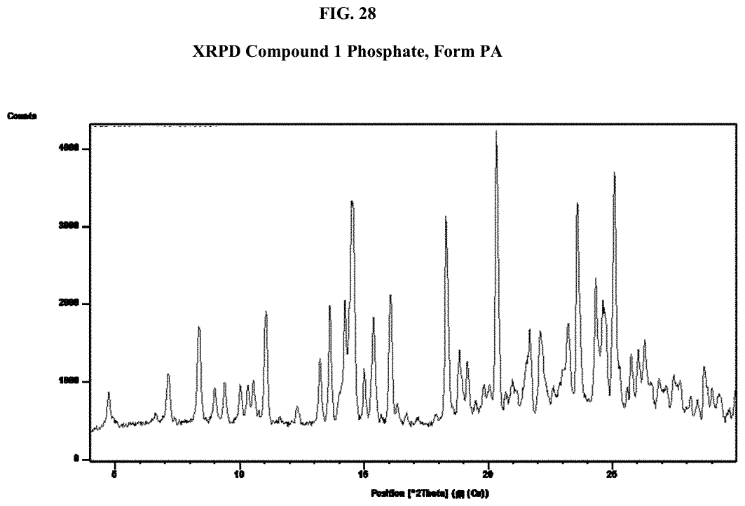

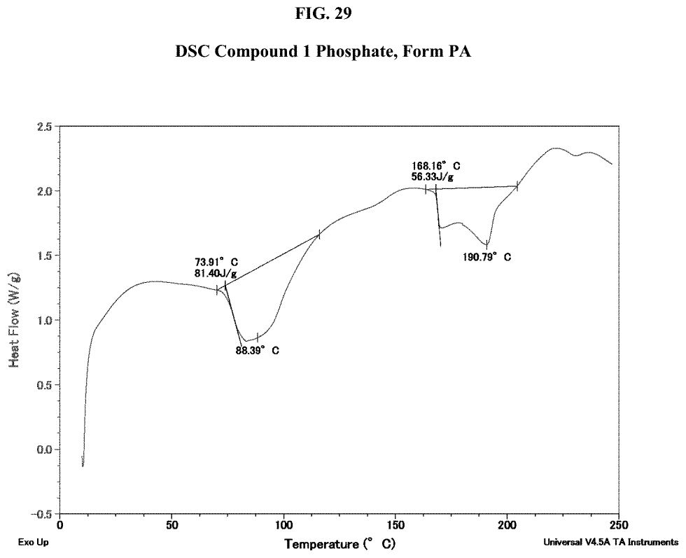

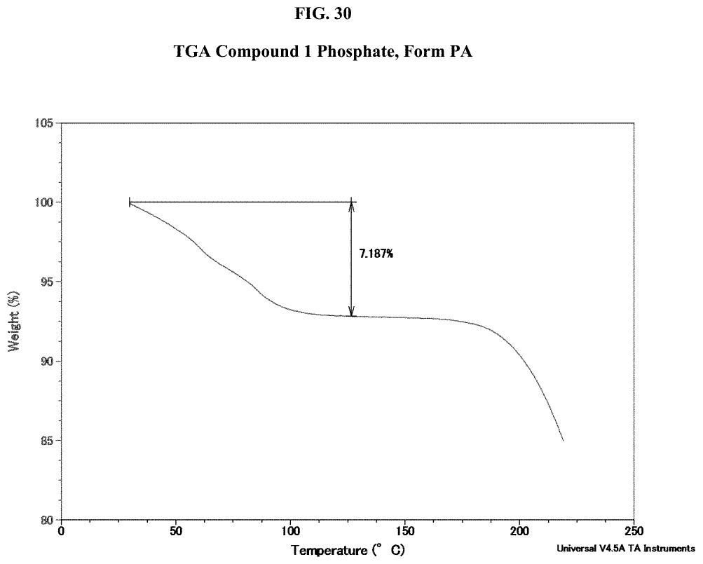

In some embodiments, Compound 1 Phosphate Form PA has an XRPD pattern with characteristic peaks as substantially shown in FIG. 28. In some embodiments, Form PA has endotherm peaks at temperatures of about 88° C. and about 191° C. In some embodiments, Compound 1 Phosphate Form PA has a DSC thermogram substantially as depicted in FIG. 29. In some embodiments, Compound 1 Phosphate Form PA has a TGA thermogram substantially as depicted in FIG. 30.

In some embodiments, Compound 1 Phosphate Form PA has at least one characteristic XRPD peak selected from about 4.7, about 6.6, about 7.1, and about 8.4 degrees 2-theta; and endotherm peaks at temperatures of about 88° C. and about 191° C. In some embodiments, Compound 1 Phosphate Form PA has at least one characteristic XRPD peak selected from about 4.7, about 6.6, about 7.1, and about 8.4 degrees 2-theta; and a DSC thermogram substantially as depicted in FIG. 29. In some embodiments, Compound 1 Phosphate Form PA has at least one characteristic XRPD peak selected from about 4.7, about 6.6, about 7.1, and about 8.4 degrees 2-theta; and a TGA thermogram substantially as depicted in FIG. 30.

In some embodiments, Compound 1 Phosphate Form PA can be isolated with a crystalline purity of at least about 80%, about 85%, about 90%, about 95%, about 96%, about 97%, about 98%, or about 99%. In some embodiments, Compound 1 Phosphate Form PA can be isolated with a crystalline purity greater than about 99%. In some embodiments, Compound 1 Phosphate Form PA can be isolated with a crystalline purity greater than about 99.9%.

Provided herein is also a process for preparing crystalline Compound 1 Phosphate, comprising precipitating Compound 1 Phosphate from a solution comprising Compound 1, phosphoric acid, and S5, wherein S5 is a solvent.

In some embodiments, provided is Compound 1 Phosphate Form PA prepared by isolating Compound 1 Phosphate Form PA from a mixture of Compound 1, phosphoric acid, and S5, wherein S5 is a solvent.

### Method of Use

The compounds and compositions, according to the method of the present invention, can be administered using any amount and any route of administration effective for treating a neurological or psychiatric disorder.

In some embodiments, the neurological or psychiatric disorder is selected from a psychosis, including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthetics, amphetamine and other psychostimulants and cocaine) psychosis, psychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both positive, negative, and cognitive symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Down syndrome, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, Parkinsonism-ALS dementia complex and basal ganglia calcification), medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias {including drug e.g. L-DOPA induced dyskinesia tremor (such as rest tremor, postural tremor, intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics), and dystonia (including generalized dystonia such as idiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)}; urinary incontinence; neuronal damage including ocular damage, retinopathy or macular degeneration of the eye, tinnitus, hearing impairment and loss, and brain edema; emesis; and sleep disorders including insomnia and narcolepsy.

In some embodiments, the neurological or psychiatric disorder is Alzheimer's Disease, Parkinson's Disease, depression, cognitive impairment, stroke, schizophrenia, Down Syndrome, or Fetal Alcohol Syndrome. In some embodiments, the neurological or psychiatric disorder is depression, bipolar disorder, pain, schizophrenia, obsessive compulsive disorder, addiction, social disorder, attention deficit hyperactivity disorder, an anxiety disorder, autism, cognitive impairments, or suicidality. In some embodiments, the neurological or psychiatric disorder is suicidality (e.g., acute suicidality, suicidal thoughts, suicidal behavior, etc.). In some embodiments, the neurological or psychiatric disorder is pain. In some embodiments, the neurological or psychiatric disorder is Alzheimer's Disease. In some embodiments, the neurological or psychiatric disorder is Parkinson's Disease.

In some embodiments, the neurological or psychiatric disorder is depression. For example, the depression is treatment-resistant depression (TRD), treatment-refractory depression, major depressive disorder (MDD), unipolar depression, bipolar depression or depression associated with another disease or disorder. In some embodiments, the depression is treatment-resistant depression. In some embodiments, the depression is MDD. In some embodiments, the depression is depression associated with another disease/disorder.

In some embodiments, the neurological or psychiatric disorder is a Depressive Disorder as defined by the Diagnostic and Statistical Manual of Mental Disorders, Fifth Edition. In some embodiments, the neurological or psychiatric disorder is disruptive mood dysregulation disorder, major depressive disorder, persistent depressive disorder (dysthymia), premenstrual dysphoric disorder, substance/medication-induced depressive disorder, depressive disorder due to another medical condition, other specified depressive disorder, or unspecified depressive disorder.

In some embodiments, the neurological or psychiatric disorder is cognitive impairment. In some embodiments, the cognitive impairment is cognitive dysfunction associated with depression, for example, major depressive disorder. In some embodiments, the neurological or psychiatric disorder is stroke. In some embodiments, the neurological or psychiatric disorder is schizophrenia. In some embodiments, the neurological or psychiatric disorder is Down syndrome. In some embodiments, the neurological or psychiatric disorder is fetal alcohol syndrome.

In some embodiments, the neurological or psychiatric disorder involves a deficit in cognition (cognitive domains as defined by the DSM-5 are: complex attention, executive function, learning and memory, language, perceptual-motor, social cognition). In some embodiments, the neurological or psychiatric disorder is associated with a deficit in dopamine signaling. In some embodiments, the neurological or psychiatric disorder is associated with basal ganglia dysfunction. In some embodiments, the neurological or psychiatric disorder is associated with dysregulated locomotor activity. In some embodiments, the neurological or psychiatric disorder is associated with impairment of prefrontal cortex functioning.

In some embodiments, the present invention provides a method of treating one or more symptoms of a neurological and/or psychiatric disorder provided herein. Such disorders include mood disorders, including bipolar I disorder, bipolar II disorder, bipolar depression, mania, cyclothymic disorder, substance/medication-induced bipolar and related disorders, bipolar and related disorder due to another medical condition, other specified bipolar and related disorder, and unspecified bipolar and related disorders; psychotic disorders, including schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), schizoaffective disorder, aggression, delirium, Parkinson's psychosis, excitative psychosis, Tourette's syndrome, and organic or NOS psychosis; depressive disorders, including disruptive mood dysregulation disorder, major depressive disorder (MDD) (including major depressive episode), dysthymia, persistent depressive disorder (dysthymia), treatment resistant depression, premenstrual dysphoric disorder, substance/medication-induced depressive disorder, depressive disorder due to another medical condition, other specified depressive disorder, and unspecified depressive disorder; anxiety disorders, including separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, other specified anxiety disorder, and unspecified anxiety disorder; stressor-related disorders, including reactive attachment disorder, disinhibited social engagement disorder, posttraumatic stress disorder (PTSD), acute stress disorder, and adjustment disorders; and other disorders including substance abuse or dependency (e.g., nicotine, alcohol, cocaine), addiction, eating disorders, behavior disorder, seizure, vertigo, epilepsy, agitation, aggression, neurodegenerative disease, Alzheimer's disease, Parkinson's disease, dyskinesias, Huntington's disease, dementia, premenstrual dysphoria; and attention deficit disorder (ADD) and neurodevelopmental disorders, including attention deficit hyperactivity disorder (ADHD)), autism, autism spectrum disorder, obsessive-compulsive disorder, pain (e.g., neuropathic pain, sensitization accompanying neuropathic pain, and inflammatory pain), fibromyalgia, migraine, cognitive impairment, movement disorder, restless leg syndrome (LS), multiple sclerosis, Parkinson's disease, Huntington's disease, dyskinesias multiple sclerosis, sleep disorder, sleep apnea, narcolepsy, excessive daytime sleepiness, jet lag, drowsy side effect of medications, insomnia, sexual dysfunction, hypertension, emesis, Lesche-Nyhane disease, Wilson's disease, and Huntington's chorea. In some embodiments, the neurological and/or psychiatric disorders include agitation and aggression. In some embodiments, the agitation and aggression are associated with Alzheimer's Disease, Parkinson's Disease, and/or autism. In some embodiments, the neurological and/or psychiatric disorders are obsessive-compulsive disorder and related disorders (e.g., body dysmorphic disorder, hoarding disorder, trichotillomania, excoriation disorder). In some embodiments, the neurological and/or psychiatric disorders are disruptive, impulse-control, and conduct disorders including oppositional defiant disorder, intermittent explosive disorder, conduct disorder, antisocial personality disorder, pyromania, kleptomania, other specified disruptive, impulse-control, and conduct disorder, unspecified disruptive, impulse-control, and conduct disorder.

In some embodiments, the present invention provides a method of treating one or more symptoms including depression (e.g., major depressive disorder or dysthymia); bipolar disorder, seasonal affective disorder; cognitive deficit; sleep related disorder (e.g., sleep apnea, insomnia, narcolepsy, cataplexy) including those sleep disorders which are produced by psychiatric conditions; chronic fatigue syndrome; anxieties (e.g., general anxiety disorder, social anxiety disorder, panic disorder); obsessive compulsive disorder; post-menopausal vasomotor symptoms (e.g., hot flashes, night sweats); neurodegenerative disease (e.g., Parkinson's disease, Alzheimer's disease and amyotrophic lateral sclerosis); manic disorder; dysthymic disorder; and obesity.

In some embodiments, a depressive disorder is associated with acute suicidality or suicide ideation. The United States Food and Drug Administration has adopted a “black box” label warning indicating that antidepressants may increase the risk of suicidal thinking and behavior in some children, adolescents and young adults (up to age 24) with a depressive disorder such as MDD. In some embodiments, a provided compound does not increase the risk of suicidal thinking and/or behavior in children, adolescents and/or young adults with a depressive disorder, e.g., with MDD. In some embodiments, the present invention provides a method of treating one or more symptoms of a depressive disorder (e.g., MDD) in children, adolescents and/or young adults without increasing the risk of suicidal thinking and/or behavior.

In some embodiments, the present invention provides a method of treating one or more symptoms including senile dementia, Alzheimer's type dementia, cognition, memory loss, amnesia/amnestic syndrome, disturbances of consciousness, coma, lowering of attention, speech disorder, Lennox syndrome, and hyperkinetic syndrome.

In some embodiments, the present invention provides a method of treating one or more symptoms of neuropathic pain, including post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use).

In some embodiments, the present invention provides a method of treating one or more symptoms including obesity; migraine or migraine headache; and sexual dysfunction, in men or women, including without limitation sexual dysfunction caused by psychological and/or physiological factors, erectile dysfunction, premature ejaculation, vaginal dryness, lack of sexual excitement, inability to obtain orgasm, and psycho-sexual dysfunction, including without limitation, inhibited sexual desire, inhibited sexual excitement, inhibited female orgasm, inhibited male orgasm, functional dyspareunia, functional vaginismus, and atypical psychosexual dysfunction.

In some embodiments, the present invention provides a method of suppressing rapid eye movement (REM) during both sleep and daytime equivalent.

In some embodiments, the present invention provides a method of suppressing or eliminating pathological or excessive REM during the night or daytime equivalent.

In some embodiments, the present invention provides a method of treating one or more symptoms including cataplexy (sudden involuntary transient bouts of muscle weakness or paralysis while awake); nighttime sleep disturbance/sleep fragmentation associated with narcolepsy or other conditions; sleep paralysis associated with narcolepsy or other conditions; hypnagogic and hypnapompic hallucinations associated with narcolepsy or other conditions; and excessive daytime sleepiness associated with narcolepsy, sleep apnea or shift work disorder and other medical conditions such as cancer, chronic fatigue syndrome and fibromyalgia.

In some embodiments, the present invention provides a medicament comprising a salt or crystalline form as described herein, and a pharmaceutically acceptable excipient.

In some embodiments, the present invention provides the use of a salt or crystalline form as described herein, in the manufacture of a medicament for the treatment of a neurological and/or psychiatric disorder.

In some embodiments, Compound 1, or a pharmaceutically acceptable salt, hydrate or solvate thereof, is admixed with a pharmaceutically acceptable excipient. In some embodiments, the salt of Compound 1 is Compound 1 Tosylate, Compound 1 Besylate, Compound 1 Hydrochloride, Compound 1 Fumarate, or Compound 1 Phosphate, or a solvate or hydrate thereof. The salt can be crystalline (e.g., a form described herein).

In some embodiments, the depression comprises treatment-resistant depression, major depressive disorder, unipolar depression, bipolar depression, acute suicidality, suicidal thoughts, suicidal behavior, or depression associated with another disease or disorder. For example, the depression is treatment-resistant depression. In some embodiments, the subject is a human.

In some embodiments, at least one of the doses is between about 0.01 mg/kg body weight and about 30 mg/kg body weight of the subject. In other embodiments, least one of the doses is between 1 mg/kg body weight and 25 mg/kg body weight of the subject. For example, at least one of the doses is between 3 mg/kg body weight and 10 mg/kg body weight of the subject.

### Pharmaceutical Compositions

Provided herein is also a pharmaceutical composition comprising Compound 1, or salts or crystalline forms thereof, and a pharmaceutically acceptable excipient. In some embodiments, the amount of compound in the compositions is such that is effective to treat, prevent, and/or manage various neurological and/or psychiatric disorders and/or symptoms in a patient. In some embodiments, the composition is formulated for administration to a patient in need of such composition. In some embodiments, the composition is formulated for oral administration to a patient.

Compositions of the present invention can be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, compositions are administered orally, intraperitoneally or intravenously. Sterile injectable forms of the compositions of this invention can be aqueous or oleaginous suspension. These suspensions can be formulated according to techniques known in the art using suitable dispersing or wetting agents and suspending agents. The sterile injectable preparation may also be a sterile injectable solution or suspension in a nontoxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium.

For this purpose, any bland fixed oil can be employed including synthetic mono-or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention can be orally administered in any orally acceptable dosage form including capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention can be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions can be formulated in a suitable ointment containing the active component suspended or dissolved in one or more carriers. Carriers for topical administration of compounds of this invention include mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions can be formulated as micronized suspensions in isotonic, pH adjusted sterile saline, or as solutions in isotonic, pH adjusted sterile saline, either with or without a preservative such as benzylalkonium chloride. Alternatively, for ophthalmic uses, the pharmaceutically acceptable compositions can be formulated in an ointment such as petrolatum.

Pharmaceutically acceptable compositions of this invention may also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

Pharmaceutically acceptable compositions of this invention can be formulated for oral administration. Such formulations can be administered with or without food. In some embodiments, pharmaceutically acceptable compositions of this invention are administered without food. In other embodiments, pharmaceutically acceptable compositions of this invention are administered with food.

The amount of compounds of the present invention that can be combined with the carrier materials to produce a composition in a single dosage form will vary depending upon a variety of factors, including the host treated and the particular mode of administration. Provided compositions should be formulated so that a dosage of between 0.01-100 mg/kg body weight/day of a compound of the present invention can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

The exact amount required will vary from subject to subject, depending on the species, age, and general condition of the subject, the severity of the infection, the particular agent, its mode of administration, and the like. The compounds of the invention can be formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The pharmaceutically acceptable compositions of this invention can be administered to humans and other animals orally, rectally, parenterally, intracisternally, intravaginally, intraperitoneal\hat{ }, topically (as by powders, ointments, or drops), bucally, as an oral or nasal spray, or the like, depending on the severity of the infection being treated. In some embodiments, the compounds of the invention can be administered orally or parenterally at dosage levels of about 0.01 mg/kg to about 50 mg/kg, e.g., from about 1 mg/kg to about 25 mg/kg, of subject body weight per day, one or more times a day, to obtain the desired therapeutic effect.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. [0141] Injectable preparations, for example, sterile injectable aqueous or oleaginous suspensions can be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This can be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration can be suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as can be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Combination Therapies

In some embodiments, the present invention provides a method of treating a neurological and/or psychiatric disorder described herein, comprising administering a compound described herein (e.g., a salt of Compound 1 (e.g., a pharmaceutically acceptable salt of Compound 1) or a crystalline form of Compound 1, or a salt thereof) in conjunction with one or more pharmaceutical agents. Suitable pharmaceutical agents that can be used in combination with the compounds of the present invention include anti-Parkinson's drugs, anti-Alzheimer's drugs, anti-depressants, anti-psychotics, anti-ischemics, CNS depressants, anti-cholinergics, and nootropics. In some embodiments, suitable pharmaceutical agents are anxiolytics.

Suitable anti-Parkinson's drugs include dopamine replacement therapy (e.g. L-DOPA, carbidopa, COMT inhibitors such as entacapone), dopamine agonists (e.g. D1 agonists, D2 agonists, mixed D1/D2 agonists; bromocriptine, pergolide, cabergoline, ropinirole, pramipexole, or apomorphine in combination with domperidone), histamine H2 antagonists, and monoamine oxidase inhibitors such as selegiline and tranylcypromine.

In some embodiments, compounds of the invention can be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone, MAO A/B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist can be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer's drugs include beta-secretase inhibitors, gamma-secretase inhibitors, HMG-CoA reductase inhibitors, NSAID's including ibuprofen, vitamin E, and anti-amyloid antibodies. In some embodiments, an anti-Alzheimer's drug is memantine.

Suitable anti-depressants and anti-anxiety agents include norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, -adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists.

Specific suitable anti-depressant and anti-anxiety agents include amitriptyline, clomipramine, doxepin, imipramine and trimipramine; amoxapine, desipramine, citalopram, escitalopram, maprotiline, nortriptyline and protriptyline; fluoxetine, fluvoxamine, paroxetine and sertraline; isocarboxazid, phenelzine, tranylcypromine and selegiline; moclobemide: venlafaxine; desvenlafaxine, duloxetine; aprepitant; bupropion, vilazodone, mirtazapine, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof. In some embodiments, suitable antidepressant and anti-anxiety agents are tianeptine, or pharmaceutically acceptable salts thereof.

Suitable anti-psychotic and mood stabilizer agents include D2 antagonists, 5HT2A antagonists, atypical antipsychotics, lithium, and anticonvulsants.

Specific suitable anti-psychotic and mood stabilizer agents include chlorpromazine, fluphenazine, haloperidol, amisulpride, chlorpromazine, perphenazine, thioridazine, trifluoperazine, aripiprazole, asenapine, clozapine, olanzapine, paliperidone, quetiapine, risperidone, ziprasidone, lurasidone, flupentixol, levomepromazine, pericyazine, perphenazine, pimozide, prochlorperazine, zuclopenthixol, olanzapine and fluoxetine, lithium, carbamazepine, lamotrigine, valproic acid and pharmaceutically acceptable salts thereof.

In some embodiments, compounds of the invention can be used in combination with other therapies. Suitable therapies include psychotherapy, cognitive behavioral therapy, electroconvulsive therapy, transcranial magnetic stimulation, vagus nerve stimulation, and deep-brain stimulation.

In some embodiments, a combination of 2 or more therapeutic agents can be administered together with the compounds of the invention. In some embodiments, a combination of 3 or more therapeutic agents can be administered with the compounds of the invention.

Other examples of agents the compounds of this invention may also be combined with include: vitamins and nutritional supplements, antiemetics (e.g. 5-HT3 receptor antagonists, dopamine antagonists, N K1 receptor antagonists, histamine receptor antagonists, cannabinoids, benzodiazepines, or anticholinergics), agents for treating Multiple Sclerosis (MS) such as beta interferon (e.g., Avonex® and Rebif®), Copaxone®, and mitoxantrone; treatments for asthma such as albuterol and Singulair®; anti-inflammatory agents such as corticosteroids, TN F blockers, IL-1 RA, azathioprine, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspoglutide), gastric inhibitory peptide analogs, DPP-4 inhibitors (vildagliptin, sitagliptin, saxagliptin, linagliptin, alogliptin), amylin analogs (pramlintide), and insulin and insulin analogs.

In some embodiments, a compound of the present invention, or a pharmaceutically acceptable salt thereof, is administered in combination with an antisense agent, a monoclonal or polyclonal antibody, or an siRNA therapeutic.

Those additional agents can be administered separately from an inventive compound-containing composition, as part of a multiple dosage regimen. Alternatively, those agents can be part of a single dosage form, mixed together with a compound of this invention in a single composition. If administered as part of a multiple dosage regime, the two active agents can be submitted simultaneously, sequentially or within a period of time from one another, normally within five hours from one another.

As used herein, the term “combination,” “combined,” and related terms refers to the simultaneous or sequential administration of therapeutic agents. For example, a compound can be administered with another therapeutic agent simultaneously or sequentially in separate unit dosage forms or together in a single unit dosage form. Accordingly, the present disclosure provides a single unit dosage form comprising a salt of Compound 1 (e.g., a pharmaceutically acceptable salt of Compound 1) or a crystalline form of Compound 1, or salt thereof, an additional therapeutic agent, and a pharmaceutically acceptable excipient.

The amount of both, an inventive compound and additional therapeutic agent (in those compositions which comprise an additional therapeutic agent as described above) that can be combined with the carrier materials to produce a single dosage form will vary depending upon the host treated and the particular mode of administration. Compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-100 mg/kg body weight/day of the additional therapeutic agent can be administered.

The amount of additional therapeutic agent present in the compositions of this invention will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

### Process for Preparing Compound 1 or Salts Thereof

Provided herein are also processes for preparing Compound 1 or a salt thereof. In some embodiments, the process is for preparing a Compound 1 p-toluenesulfonic acid salt. The processes for preparing Compound 1 or a salt thereof provided herein have certain advantages over the processes currently disclosed in the art. For example, the processes described herein demonstrate good scalability, yields, and stereochemical selectivity.

In some embodiments, the processes described herein include “enantioselective synthesis”, also called “asymmetric synthesis.” Enantioselective synthesis is defined by IUPAC as: a chemical reaction (or reaction sequence) in which one or more new elements of chirality are formed in a substrate molecule and which produces the stereoisomeric (enantiomeric or diastereoisomeric) products in unequal amounts. It is the synthesis of a compound by a method that favors the formation of a specific enantiomer or diastereomer. The methods disclosed herein may provide benefits related to cost, scale-up, and environmental impact compared to a process requiring separation of the stereoisomeric (enantiomeric or diastereoisomeric) products by using chiral column HPLC, which could result in disposing half of the products as undesired isomers.

In some embodiments, the processes described herein include an approach using “enantioselective catalysis” in enantioselective synthesis. In general, enantioselective catalysis (known traditionally as asymmetric catalysis) are chiral coordination complexes. In some embodiments, the catalysts are rendered chiral by using chiral ligands. An example of enantioselective synthesis is asymmetric hydrogenation, which is used to reduce unsaturated bond of a wide variety of functional groups such as carbonyl groups or olefins (N. Jacobsen, Eric; Pfaltz, Andreas; Yamamoto, Hisashi (1999). Comprehensive asymmetric catalysis 1-3. Berlin: Springer. ISBN 9783540643371; M. Heitbaum; F. Glorius; I. Escher (2006). “Asymmetric Heterogeneous Catalysis”. Angewandte Chemie International Edition. 45 (29): 4732-4762; Asymmetric Catalysis on Industrial Scale, (Blaser, Schmidt), Wiley-VCH, 2004), which are each incorporated herein. The process for preparing Compound 1 or a salt thereof can comprise:
a) reacting Compound 1a-G having the formula:

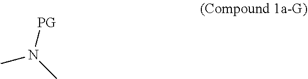

wherein PG is a suitable protecting group (e.g., Boc) with a lithiating agent to form Compound 2a-G, wherein Compound 2a-G is a lithiated Compound 1a-G;
b) reacting Compound 2a-G with Compound 3a having the formula:

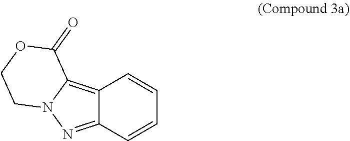 [SMILES: O=C1OCCn2nc3ccccc3c21]

to form Compound 4a-G having the formula:

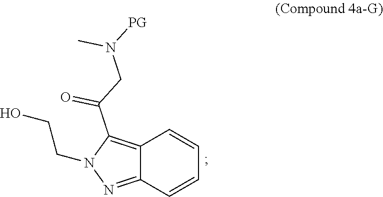

c) reacting Compound 4a-G with a reducing agent to form Compound 5a-G having the formula:

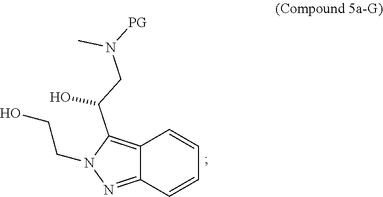

d) reacting Compound 5a-G with a suitable leaving group (e.g., p-toluenesulfonyl chloride) to form Compound 6a-G having the formula:

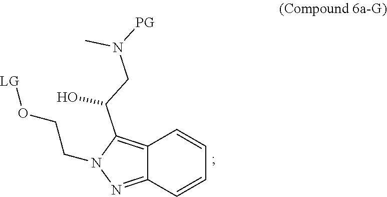

wherein LG is a leaving group (e.g., p-toluenesulfonyl);
e) reacting Compound 6a-G with a suitable base (e.g., 2,3,4,6,7,8,9,10-octahydropyrimido[1,2-a]azepine (1, 8-Diazabicyclo[5,4,0]undec-7-ene; DBU)) to form Compound 7a-G having the formula:

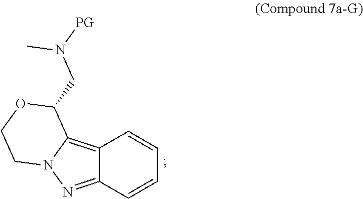

and
f) reacting Compound 7a-G with an acid (e.g., p-toluenesulfonic acid) to form Compound 1 or a pharmaceutically acceptable salt, hydrate, or solvate thereof.

In some embodiments, Compound 7a-G can be represented as follows:

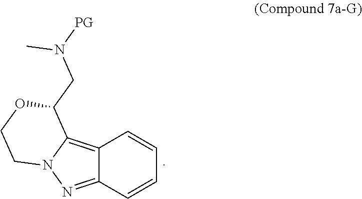

Suitable protecting group (PG) includes alkoxycarbonyl or acyl groups such as R¹O(C═O) or R¹(C═O), wherein R¹ is alkyl (e.g., C₁₋₆ alkyl) or alkylene-aryl (C₁₋₆ alkylene-(C₆₋₁₀ membered aryl)). For example, the protecting group can be tert-butyloxycarbonyl (BOC) or fluorenylmethyloxycarbonyl protecting group (Fmoc). Various acids and deprotection conditions can be used to remove the protecting group. The use of toluenesulfonic acid enables the one-pot reaction of Compound 7a-G to Compound 1 Tosylate. Suitable leaving group (LG) includes halides such as Cl, Br, and I, and sulfonate esters such as triflate (TfO) and mesylate (MsO).

The process for preparing Compound 1 or a salt thereof can comprise:
a) reacting Compound 1a having the formula:

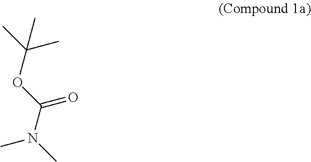

with a lithiating agent to form Compound 2a, wherein Compound 2a is a lithiated Compound 1a (e.g., lithiated tert-butoxycarbonyldimethylamine);
b) reacting Compound 2a with Compound 3a having the formula:

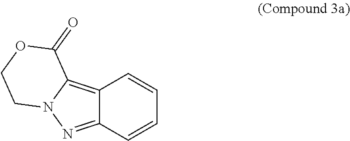 [SMILES: O=C1OCCn2nc3ccccc3c21]

to form Compound 4a having the formula:

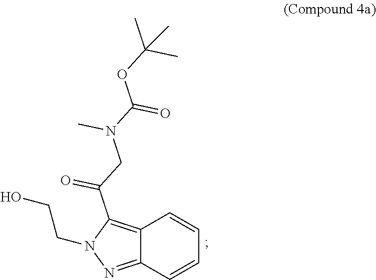 [SMILES: CN(CC(=O)c1c2ccccc2nn1CCO)C(=O)OC(C)(C)C]

c) reacting Compound 4a with a reducing agent to form Compound 5a having the formula:

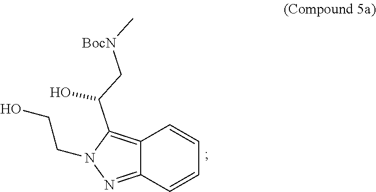 [SMILES: CN(C[C@@H](O)c1c2ccccc2nn1CCO)C(=O)OC(C)(C)C]

d) reacting Compound 5a with p-toluenesulfonyl chloride to form Compound 6a having the formula:

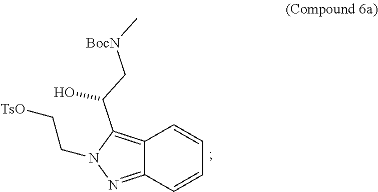 [SMILES: Cc1ccc(S(=O)(=O)OCCn2nc3ccccc3c2[C@H](O)CN(C)C(=O)OC(C)(C)C)cc1]

e) reacting Compound 6a with a suitable base (e.g., 2,3,4,6,7,8,9,10-octahydropyrimido[1,2-a]azepine (1, 8-Diazabicyclo[5,4,0]undec-7-ene; DBU) to form Compound 7a having the formula:

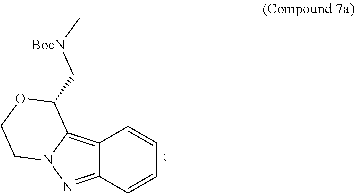 [SMILES: CN(C[C@H]1OCCn2nc3ccccc3c21)C(=O)OC(C)(C)C]

and
f) reacting Compound 7a with an acid (e.g., p-toluenesulfonic acid) to form Compound 1 or a pharmaceutically acceptable salt (Compound 1 Tosylate), hydrate, or solvate thereof.

In some embodiments, Compound 7a can be represented as follows:

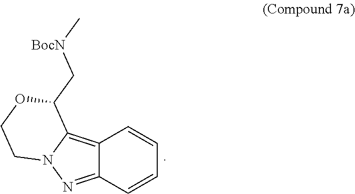 [SMILES: CN(C[C@H]1OCCn2nc3ccccc3c21)C(=O)OC(C)(C)C]

In some embodiments, the process for preparing Compound 3a comprises:
reacting Compound 8a having the formula:

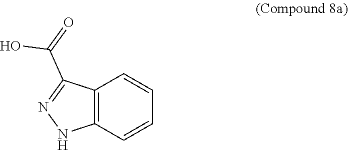 [SMILES: O=C(O)c1n[nH]c2ccccc12]

with 2-bromoethan-1-ol to form Compound 9a having the formula:

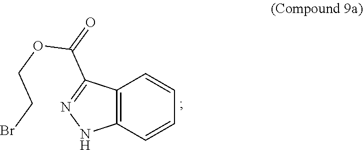 [SMILES: O=C(OCCBr)c1n[nH]c2ccccc12]

and
reacting Compound 9a with a base to form Compound 3a.

In some embodiments, Compound 2a is lithiated tert-butoxycarbonyldimethylamine. Lithiated tert-butoxycarbonyldimethylamine (Compound 2a) can be prepared by a process that includes reacting tert-butoxycarbonyldimethylamine (Compound 1a) with a lithiating agent. In some embodiments, the lithiating agent is an alkyllithium reagent. In some embodiments, the lithiating reagent is sec-butyllithium. In some embodiments, the lithiating agent is t-butyllithium. The reacting can be carried out in the presence of a ligand and S6a, wherein S6a is a solvent. The ligand can be an amine. For example, suitable ligands include ligands that can form a lithium agent for selective lithiation. In some embodiments, the ligand is tetramethylethylenediamine. The solvent S6a can be a polar aprotic solvent. In some embodiments, the polar aprotic solvent is an ether (e.g., diethyl ether and tetrahydrofuran). In some embodiments, the polar aprotic solvent is tetrahydrofuran. In some embodiments, the reacting of Compound 1a with a lithiating agent is carried out at a temperature of about −120° C. to about −40° C. In some embodiments, the reacting of Compound 1a with a lithiating agent is carried out at a temperature of about −100° C. to about −60° C., e.g., between about −78° C. and about −70° C. In some embodiments, between about 0.5 and about 5 molar equivalents of lithiating agent are used per molar equivalent of Compound 1a. In some embodiments, between about 0.5 and about 2 molar equivalents of lithiating agent are used per molar equivalent of Compound 1a. In some embodiments, between about 0.5 and about 1.5 (e.g., about 1) molar equivalents of lithiating agent are used per molar equivalent of Compound 1a. In some embodiments, wherein between about 0.5 and about 5 molar equivalents of the ligand are used per molar equivalent of Compound 1a. In some embodiments, between about 0.5 and about 3 (e.g., about 1 or about 2) molar equivalents of ligand are used per molar equivalent of Compound 1a.

In some embodiments, the process for preparing 3,4-dihydro-1H-[1,4]oxazino[4,3-b]indazol-1-one (Compound 3a) comprises reacting 2-bromoethyl 1H-indazole-3-carboxylate (Compound 9a) with B2a to form Compound 3a, wherein B2a is an alkali metal base. In some embodiments, the base B2a is an alkali metal carbonate. For example, B2a can be potassium carbonate. In some embodiments, the reacting of Compound 9a with B2a is carried out in the presence of S7a, wherein S7a is a solvent. For example, solvent S7a is a polar aprotic solvent. Example of polar aprotic solvent includes N-methylpyrrolidone. In some embodiments, the reacting of Compound 9a with B2a is carried out at a temperature of about 50° C. to about 120° C. For example, the reacting of Compound 9a with B2a is carried out at a temperature of about 70° C. to about 100° C. (e.g., about 90° C.). In some embodiments, between about 0.5 and about 2 molar equivalents of B2a are used per molar equivalent of Compound 9a. For example, between about 1 and about 1.5 (e.g., about 1.2) molar equivalents of B2a are used per molar equivalent of Compound 9a. The work up of the reaction mixture can include contacting the mixture with an acid such as acetic acid.

In some embodiments, the process of preparing 2-bromoethyl 1H-indazole-3-carboxylate (Compound 9a) comprises reacting 1H-indazole-3-carboxylic acid (Compound 8a) with 2-bromoethanol, a chlorinating agent, and an amine compound. For example, the chlorinating agent is thionyl chloride. The amine can be a tertiary amine. For example, the tertiary amine is trimethylamine. In some embodiments, the reacting of Compound 8a with 2-bromoethanol, a chlorinating agent, and an amine compound, is carried out in the presence of S8a, wherein S8a is a solvent. The solvent S8a can be a polar aprotic solvent. The polar aprotic solvent of S8a can be dimethylformamide, 1,2-dichloroethane, or a mixture thereof. The reacting of Compound 8a with a chlorinating agent is carried out to form a first mixture. For example, the reacting to form the first mixture can be carried out at a temperature of about 50° C. to about 100° C. to form a first mixture. The reacting of Compound 8a with a chlorinating agent can be carried out at a temperature of about 60° C. to about 80° C. (e.g., about 70° C.) to form a first mixture. In some embodiments, 2-bromoethanol and the amine are added to the first mixture at a temperature of about 20° C. to about 40° C. In some embodiments, between about 1 and about 2 molar equivalents of chlorinating agent are used per molar equivalent of Compound 8a. For example, between about 1 and about 1.5 (e.g., about 1.2) molar equivalents of chlorinating agent are used per molar equivalent of Compound 8a. In some embodiments, between about 1 and about 2 molar equivalents of 2-bromoethanol are used per molar equivalent of Compound 8a. For example, between about 1.5 and about 2 (e.g., about 1.8) molar equivalents of 2-bromoethanol are used per molar equivalent of Compound 8a. In some embodiments, between about 1 and about 4 molar equivalents of amine are used per molar equivalent of Compound 8a. For example, between about 2 and about 3 (e.g., about 2.4 or about 2.5) molar equivalents of amine are used per molar equivalent of Compound 8a.

tert-Butyl (2-(2-(2-hydroxyethyl)-2H-indazol-3-yl)-2-oxoethyl)(methyl)carbamate (Compound 4a) can be prepared by a process that includes reacting Compound 3a with lithiated tert-butoxycarbonyldimethylamine (Compound 2a). The reacting can be carried out in the presence of S5a, wherein S5a is a solvent. The solvent S5a can be a polar aprotic solvent. In some embodiments, the polar aprotic solvent is an ether (e.g., diethyl ether and tetrahydrofuran). In some embodiments, the polar aprotic solvent is tetrahydrofuran. In some embodiments, the reacting of Compound 3a with lithiated tert-butoxycarbonyldimethylamine is carried out at a temperature of about −120° C. to about 10° C. In some embodiments, the reacting of Compound 3a with lithiated tert-butoxycarbonyldimethylamine is carried out at a temperature of about −100° C. to about −60° C., e.g., between about −78° C. to about −65° C., between about −45° C. to about −50° C., between about 0° C. to about −3° C., and between −45° C. to about −55° C. In some embodiments, a solution of Compound 3a is added to solution of Compound 2a. In some embodiments, a solution of Compound 2a is added to a solution of Compound 3a. In some embodiments, between about 1 and about 5 molar equivalents of Compound 2a are used per molar equivalent of Compound 3a. In some embodiments, between about 1 and about 2 molar equivalents of Compound 2a are used per molar equivalent of Compound 3a.

The reacting of Compound 3a with Compound 2a can be performed in a continuous flow process. The flow processes described in Examples 9 and 10 were developed to address certain limitations of performing the reactions in batches (e.g., the process for synthesizing Compound 4a described in Example 8). The nature of the reactions call for process control in terms of temperature and heat transfer, immediate transfer, reaction time, mixing control, etc., which can lead to challenges for scale-up of the batch process, including:

1. It is difficult to handle the exothermic and instant reactions in batch;

2. The batch process typically left about 10-15% of the unreacted Compound 3a in lab scale, and this conversion dropped to less than 50% at scale up, providing very low yields on scale;

3. 10-20% yield loss was observed while scaling up to kilo-lab scale, which is due to degradation of lithiated Compound 1a during preparation and transferring;

4. A set of two neighboring cryogenic reactors is required, which causes logistical problems at scale-up.

In some embodiments, the flow process provides superior yield, scalability, reaction times, and consistency. For example, the flow process provides a 90-95% yield of Compound 4a (see e.g., Example 10), compared to a 60-70% yield of Compound 4a in the batch process. Further, the batch process leads to an additional 10-15% yield loss during scale-up. In contrast, the high yield of the flow process is maintained when scaling up. The purity after isolation for the flow process was 100%, compared to 99.3% for the batch process. Finally, the total residence time for each reaction was less than a minute for the flow process, while the batch process requires a 1-3 h reaction time.

The cryogenic flow reactor process is compatible with the exothermic and instant reactions described above. The flow reactions provide no accumulation of lithiated Compound 1a (i.e., Compound 2a), which allows for minimal degradation. In contrast, the batch process requires accumulation of Compound 2a, resulting in significant degradation. Because the two flow reactors are connected, the process eliminates the transfer of Compound 2a, further limiting degradation.

Finally, the steady-state operation of the flow process provides consistent yields and purity of the desired product, while there is variation between batches in the batch process.

In some embodiments, the continuous flow process comprises:

adding Compound 1a to a first reactor and contacting said Compound 1a with a lithiating agent to provide Compound 2a; and

transferring said Compound 2a to a second reactor and contacting Compound 2a with Compound 3a to provide Compound 4a.

In some embodiments, the continuous flow process is carried out at an average temperature between about −80° C. and about −20° C. In some embodiments, the continuous flow process is carried out at an average temperature between about −50° C. and about −30° C. In some embodiments, the continuous flow process is carried out at an average temperature between about −42° C. and about −36° C. In some embodiments, the continuous flow process has a total residence time between about 0.2 minutes and about 10 minutes. In some embodiments, the continuous flow process has a total residence time between about 0.5 minutes and about 2 minutes. In some embodiments, the continuous flow process has a total residence time between about 0.2 minutes and about 5 minutes. In some embodiments, the total residence time is measured from adding Compound 1a to the first reactor to providing Compound 4a. In some embodiments, the lithiation reaction (e.g., the contacting of Compound 1a with the lithiating agent) of the continuous flow process has a residence time of less than a minute. In some embodiments, the reaction of Compound 3a (e.g., the contacting of Compound 2a with Compound 3a) of the continuous flow process has a residence time of less than a minute. In some embodiments, each reaction (e.g., the lithiation reaction and the reaction of Compound 3a) of the continuous flow process has a residence time between about 5 seconds and about 1 minute.

In some embodiments, the lithiating agent is an alkyllithium reagent. In some embodiments, the lithiating agent is sec-butyllithium. The contacting of Compound 1a with the lithiating agent can be carried out in the presence of a ligand and S6b, wherein S6b is a solvent. In some embodiments, S6b is a mixture of a polar aprotic solvent and a non-polar solvent. In some embodiments, S6b is a mixture of tetrahydrofuran and toluene. In some embodiments, the ligand is an amine. In some embodiments, the ligand is tetramethylethylenediamine. In some embodiments, between about 0.5 and about 5 molar equivalents of the lithiating agent are used per molar equivalent of Compound 1a. In some embodiments, between about 0.5 and about 2 molar equivalents of the lithiating agent are used per molar equivalent of Compound 1a. In some embodiments, about 1 molar equivalent of the lithiating agent is used per molar equivalent of Compound 1a. In some embodiments, between about 1 and about 5 molar equivalents of the ligand are used per molar equivalent of Compound 1a. In some embodiments, between about 3 and about 4 molar equivalents of the ligand are used per molar equivalent of Compound 1a. In some embodiments, a flow rate between about 0.5 mmol and about 5 mmol of Compound 1a per minute is used in the continuous flow process. In some embodiments, a flow rate between about 0.5 mmol and about 1 mmol of Compound 1a per minute is used in the continuous flow process. In some embodiments, a flow rate between about 2 mmol and about 5 mmol of Compound 1a per minute is used in the continuous flow process. In some embodiments, a flow rate between about 3 mmol and about 4 mmol of Compound 1a per minute is used in the continuous flow process. In some embodiments, a flow rate between about 0.5 mmol and about 5 mmol of the lithiating agent per minute is used in the continuous flow process. In some embodiments, a flow rate between about 0.5 mmol and about 1 mmol of the lithiating agent per minute is used in the continuous flow process. In some embodiments, a flow rate between about 2 mmol and about 5 mmol of the lithiating agent per minute is used in the continuous flow process. In some embodiments, a flow rate between about 3 mmol and about 4 mmol of the lithiating agent per minute is used in the continuous flow process.

The contacting of Compound 3a with Compound 2a can be carried out in the presence of S5b, wherein S5b is a solvent. In some embodiments, S5b is a polar aprotic solvent. In some embodiments, the polar aprotic solvent of S5b is tetrahydrofuran. In some embodiments, S5b is a mixture of a polar aprotic solvent and a non-polar solvent. In some embodiments, S5b is a mixture of a tetrahydrofuran and toluene. In some embodiments, between about 1 and about 5 molar equivalents of Compound 2a are used per molar equivalent of Compound 3a. In some embodiments, between about 1 and about 2 molar equivalents of Compound 2a are used per molar equivalent of Compound 3a. In some embodiments, a flow rate between about 0.25 mmol and about 5 mmol of Compound 3a per minute is used in the continuous flow process. In some embodiments, a flow rate between about 1 mmol and about 3 mmol of Compound 3a per minute is used in the continuous flow process. In some embodiments, a flow rate between of about 2 mmol of Compound 3a per minute is used in the continuous flow process. In some embodiments, a flow rate between of about 0.5 mmol of Compound 3a per minute is used in the continuous flow process.

The process for preparing Compound 4a can further include addition of an acid in a solvent. In some embodiments, the solvent is a polar aprotic solvent. The solvent can be a solvent miscible with tetrahydrofuran. In some embodiments, the process for preparing Compound 4a can further include addition of an acid in a polar aprotic solvent after the reaction of Compound 3a with Compound 2a. In some embodiments, the acid is an acid with mild property to minimize heat of neutralization. In some embodiments, the acid is acetic acid and the polar aprotic solvent is tetrahydrofuran. The process can include addition of a second acid. In some embodiments, the acid is hydrochloric acid, e.g., aqueous hydrochloric acid. In some embodiments, Compound 4a is extracted in an organic solvent comprising cyclopentylmethylether, toluene, or a mixture thereof. In some embodiments, Compound 4a is extracted in an organic solvent comprising cyclopentylmethylether. In some embodiments, Compound 4a is extracted in an organic solvent comprising toluene.

tert-Butyl (R)-(2-hydroxy-2-(2-(2-hydroxyethyl)-2H-indazol-3-yl)ethyl)(methyl)carbamate (Compound 5a) can be prepared by a process that includes reacting tert-butyl (2-(2-(2-hydroxyethyl)-2H-indazol-3-yl)-2-oxoethyl)(methyl)carbamate (Compound 4a) with a reducing agent. The reaction can be carried out as an enantioselective synthesis or asymmetric synthesis. However, in some embodiments, the reaction may not necessarily be enantioselective synthesis, e.g., the reducing agent may be achiral, and after obtaining the racemic Compound 5ab, chiral resolution (such as chiral column HPLC separation) may be used to obtain Compound 5a or Compound 5b. In some embodiments, the reducing agent is NaBH₄, LiBH₄, or LAH. In some embodiments, the reducing reagent can comprise chiral ligand(s) in the context of enantioselective reduction. In some embodiments, the reducing reagent comprises hydrogen in the presence of a transition metal catalyst. In some embodiments, the reducing reagent comprises hydrogen in the presence of a transition metal catalyst with chiral ligand(s) (a chiral coordination complex) in the context of enantioselective catalysis or asymmetric hydrogenation. Typical catalyst for asymmetric reduction can be found in, e.g., Angew. Chem. Int. Ed. 2001, 40, 40-73 and J. Am. Chem. Soc. 2011, 10696, each of which is incorporated herein in its entirety by reference. In some embodiments, the transition metal catalyst is a ruthenium catalyst (e.g., ruthenium catalyst with chiral ligand(s)). In some embodiments, the ruthenium catalyst is (R)-RUCY-XylBINAP (RuCl[(R)-daipena][(R)-xylbinap]) to afford Compound 5a. In some embodiments, the ruthenium catalyst is (S)-RUCY-XylBINAP (RuCl[(S)-daipena][(S)-xylbinap]) to afford another enantiomer Compound 5b. The reacting can be carried out in the presence of B1a, wherein B1a is a base (e.g., an alkali metal alkoxide base). The B1a is a potassium alkoxide base. In some embodiments, B1a is potassium tert-butoxide. In some embodiments, the base is DBU. The reacting can be carried out in the presence of S4a, wherein S4a is a solvent. The solvent S4a can be a non-polar solvent. In some embodiments, the non-polar solvent is toluene. In some embodiments, S4a is an alcohol (e.g., isopropyl alcohol), halogenated solvent (e.g., dichloromethane), ether (e.g., tetrahydrofuran), or a mixture thereof. In some embodiments, the pressure of hydrogen is between about 1 bar and about 10 bar. In some embodiments, between about 0.001 and about 0.02 molar equivalents of transition metal catalyst are used per molar equivalent of Compound 4a. In some embodiments, between about 0.0001 and about 0.05 molar equivalents of transition metal catalyst are used per molar equivalent of Compound 4a. In some embodiments, between about 0.001 and about 0.02 molar equivalents of transition metal catalyst are used per molar equivalent of Compound 4a. In some embodiments, between about 0.05 and about 0.5 molar equivalents of alkali metal alkoxide base are used per molar equivalent of Compound 4a. In some embodiments, between about 0.1 and about 0.3 molar equivalents of alkali metal alkoxide base are used per molar equivalent of Compound 4a.

In some embodiments, Compound 5a is not isolated. Compound 4a can be converted to Compound 5a in greater than about 99% yield and its enantioselectivity is about 99% ee.

(R)-2-(3-(2-((tert-Butoxycarbonyl)(methyl)amino)-1-hydroxyethyl)-2H-indazol-2-yl)ethyl 4-methylbenzenesulfonate (Compound 6a) can be prepared by a process that includes reacting tert-butyl (R)-(2-hydroxy-2-(2-(2-hydroxyethyl)-2H-indazol-3-yl)ethyl)(methyl)carbamate (Compound 5a) with p-toluenesulfonyl chloride. In some embodiments, the reacting is carried out in the presence of trimethylamine hydrochloride and N-methylmorpholine. In some embodiments, the reacting is carried out in the presence of 2-morpholinoethan-1-amine. For example, the process of preparing Compound 6a can further include the addition of 2-morpholinoethan-1-amine after reaction of Compound 5a and p-toluenesulfonyl chloride is complete. Without being limited to a particular theory, 2-morpholinoethan-1-amine is believed to trap excess of p-toluenesulfonyl chloride and the addition of 2-morpholinoethan-1-amine is typically added after the reaction of Compound 5a and toluenesulfonyl chloride is complete. In some embodiments, the reacting is carried out in the presence of trimethylamine hydrochloride, N-methylmorpholine, and 2-morpholinoethan-1-amine. In some embodiments, the reacting is carried out in the presence of trimethylamine hydrochloride, N-methylmorpholine, and after the reaction completion, 2-morpholinoethan-1-amine is added. The reacting can be carried out in the presence of S3a, wherein S3a is a solvent. The solvent S3a can be a non-polar solvent. In some embodiments, the non-polar solvent is toluene. In some embodiments, reacting of Compound 5a with p-toluenesulfonyl chloride is carried out at a temperature of about −10° C. to about 40° C. In some embodiments, the reacting of Compound 5a with p-toluenesulfonyl chloride is carried out at a temperature of about 10° C. to about 30° C. In some embodiments, between about 1 and about 5 molar equivalents of p-toluenesulfonyl chloride are used per molar equivalent of Compound 5a. In some embodiments, between about 1 and about 2 molar equivalents of p-toluenesulfonyl chloride are used per molar equivalent of Compound 5a. In some embodiments, between about 0.02 and about 0.2 molar equivalents of trimethylamine hydrochloride are used per molar equivalent of Compound 5a. In some embodiments, between about 0.05 and about 0.1 molar equivalents of trimethylamine hydrochloride are used per molar equivalent of Compound 5a. In some embodiments, between about 1 and about 5 molar equivalents of N-methylmorpholine are used per molar equivalents of Compound 5a. In some embodiments, between about 1 and about 2 molar equivalents of N-methylmorpholine are used per molar equivalents of Compound 5a. In some embodiments, between about 0.05 and about 0.5 molar equivalents of 2-morpholinoethan-1-amine are used per molar equivalent of Compound 5a. In some embodiments, between about 0.1 and about 0.3 molar equivalents of 2-morpholinoethan-1-amine are used per molar equivalent of Compound 5a. In some embodiments, the process of preparing Compound 6a include reacting Compound 5a with p-toluenesulfonyl chloride in the presence of trimethyl amine and N-methylmorpholine, and subsequently adding 4-(2-aminoethylethyl)morpholine.

tert-Butyl (R)-((3,4-dihydro-1H-[1,4]oxazino[4,3-b]indazol-1-yl)methyl)(methyl)carbamate (Compound 7a) can be prepared by a process that includes reacting (R)-2-(3-(2-((tert-butoxycarbonyl)(methyl)amino)-1-hydroxyethyl)-2H-indazol-2-yl)ethyl 4-ethylbenzenesulfonate (Compound 6a) with 2,3,4,6,7,8,9,10-octahydropyrimido[1,2-a]azepine (1,8-diazabicyclo[5,4,0]undec-7-ene; DBU). The reacting can be carried out in the presence of a S2a, wherein S2a is a solvent. The solvent S2a can be a protic solvent, non-polar solvent, or a mixture thereof. In some embodiments, the polar protic solvent of S2a is water. In some embodiments, the protic solvent of S2a is toluene. In some embodiments, the reacting of Compound 6a with 2,3,4,6,7,8,9,10-octahydropyrimido[1,2-a]azepine is carried out at a temperature of about 30° C. to about 90° C. In some embodiments, the reacting of Compound 6a with 2,3,4,6,7,8,9,10-octahydropyrimido[1,2-a]azepine is carried out at a temperature of about 50° C. to about 70° C. In some embodiments, between about 1 and about 5 molar equivalents of 2,3,4,6,7,8,9,10-octahydropyrimido[1,2-a]azepine are used per molar equivalent of Compound 6a. In some embodiments, between about 1 and about 2 molar equivalents of 2,3,4,6,7,8,9,10-octahydropyrimido[1,2-a]azepine are used per molar equivalent of Compound 6a.

The process to prepare Compound 1 can include reacting tert-butyl (R)-((3,4-dihydro-1H-[1,4]oxazino[4,3-b]indazol-1-yl)methyl)(methyl)carbamate (Compound 7a) with an acid (e.g., p-toluenesulfonic acid). Suitable acids include acids that can remove the Boc or other protecting groups. In some embodiments, the p-toluenesulfonic acid is a p-toluenesulfonic acid mono-hydrate. The reacting can be carried out in the presence of S1a, wherein S1a is a solvent. The solvent S1a can be a polar aprotic solvent, non-polar solvent, protic solvent, or a mixture thereof. The solvent S1a can be a polar aprotic solvent, non-polar solvent, or a mixture thereof. In some embodiments, the polar aprotic solvent of S1a is acetonitrile, chloroform, acetone, or a mixture thereof. In some embodiments, the polar aprotic solvent of S1a is acetonitrile. In some embodiments, the non-polar solvent of S1a is toluene. In some embodiments, the protic solvent is water, methanol, or a mixture thereof. In some embodiments, the reacting of Compound 7a with p-toluene sulfonic acid is carried out at a temperature of about 50° C. to about 100° C. In some embodiments, the reacting of Compound 7a with p-toluenesulfonic acid is carried out at a temperature of about 70° C. to about 90° C. In some embodiments, between about 1 and about 5 molar equivalents of p-toluenesulfonic acid are used per molar equivalent of Compound 7a. In some embodiments, between about 1 and about 2 molar equivalents of p-toluenesulfonic acid are used per molar equivalent of Compound 7a.

In some embodiments, the total yield from Compound 3a, to Compound 4a, to Compound 5a, to Compound 6a, and to Compound 1 Tosylate is about 70%. The chemical purity of Compound 1 or Compound 1 Tosylate is greater than about 99% and its enantioselectivity is greater than about 99% ee.

In some embodiments, Compound 1 is isolated as Compound 1 Tosylate. In some embodiments, Compound 1 is isolated as Compound 1 Besylate. In some embodiments, Compound 1 is isolated as Compound 1 Hydrochloride. In some embodiments, Compound 1 is isolated as Compound 1 Fumarate. In some embodiments, Compound 1 is isolated as Compound 1 Phosphate.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 7a-G with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 6a-G with a base to produce Compound 7a-G; and
reacting Compound 7a-G with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 5a-G with p-toluenesulfonyl chloride to produce Compound 6a-G;
reacting Compound 6a-G with a base to produce Compound 7a-G; and
reacting Compound 7a-G with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 4a-G with a reducing agent to produce Compound 5a-G;
reacting Compound 5a-G with p-toluenesulfonyl chloride to produce Compound 6a-G;
reacting Compound 6a-G with a base to produce Compound 7a-G; and
reacting Compound 7a-G with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 2a-G with Compound 3a to produce Compound 4a-G;
reacting Compound 4a-G with a reducing agent to produce Compound 5a-G;
reacting Compound 5a-G with p-toluenesulfonyl chloride to produce Compound 6a-G;
reacting Compound 6a-G with a base to produce Compound 7a-G; and
reacting Compound 7a-G with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 1a-G with a lithiating agent to produce Compound 2a-G;
reacting Compound 2a-G with Compound 3a to produce Compound 4a-G;
reacting Compound 4a-G with a reducing agent to produce Compound 5a-G;
reacting Compound 5a-G with p-toluenesulfonyl chloride to produce Compound 6a-G;
reacting Compound 6a-G with a base to produce Compound 7a-G; and
reacting Compound 7a-G with an acid to produce Compound 1 acid salt (e.g., Compound 1 Tosylate, Compound 1 Besylate, Compound 1 Hydrochloride, Compound 1 Fumarate, or Compound 1 Phosphate).

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 1a-G with a lithiating agent to produce Compound 2a-G;
reacting Compound 8a with 2-bromoethane to produce Compound 9a;
reacting Compound 9a with an alkali metal base to produce Compound 3a;
reacting Compound 2a-G with Compound 3a to produce Compound 4a-G;
reacting Compound 4a-G with a reducing agent to produce Compound 5a-G;
reacting Compound 5a-G with p-toluenesulfonyl chloride to produce Compound 6a-G;
reacting Compound 6a-G with a base to produce Compound 7a-G; and
reacting Compound 7a-G with an acid to produce Compound 1 acid salt (e.g., Compound 1 Tosylate, Compound 1 Besylate, Compound 1 Hydrochloride, Compound 1 Fumarate, or Compound 1 Phosphate).

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 7a with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 6a with a base to produce Compound 7a; and
reacting Compound 7a with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 5a with p-toluenesulfonyl chloride to produce Compound 6a;
reacting Compound 6a with a base to produce Compound 7a; and
reacting Compound 7a with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 4a with a reducing agent to produce Compound 5a;
reacting Compound 5a with p-toluenesulfonyl chloride to produce Compound 6a;
reacting Compound 6a with a base to produce Compound 7a; and
reacting Compound 7a with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 2a with Compound 3a to produce Compound 4a;
reacting Compound 4a with a reducing agent to produce Compound 5a;
reacting Compound 5a with p-toluenesulfonyl chloride to produce Compound 6a;
reacting Compound 6a with a base to produce Compound 7a; and
reacting Compound 7a with an acid to produce Compound 1 acid salt.

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 1a with a lithiating agent to produce Compound 2a;
reacting Compound 2a with Compound 3a to produce Compound 4a;
reacting Compound 4a with a reducing agent to produce Compound 5a;
reacting Compound 5a with p-toluenesulfonyl chloride to produce Compound 6a;
reacting Compound 6a with a base to produce Compound 7a; and
reacting Compound 7a with an acid to produce Compound 1 acid salt (e.g., Compound 1 Tosylate, Compound 1 Besylate, Compound 1 Hydrochloride, Compound 1 Fumarate, or Compound 1 Phosphate).

The process of preparing Compound 1 acid salt (e.g., Compound 1 Tosylate, Compound 1 Besylate, Compound 1 Hydrochloride, Compound 1 Fumarate, or Compound 1 Phosphate) can comprise:
reacting Compound 1a with a lithiating agent to produce Compound 2a;
reacting Compound 8a with 2-bromoethane to produce Compound 9a;
reacting Compound 9a with an alkali metal base to produce Compound 3a;
reacting Compound 2a with Compound 3a to produce Compound 4a;
reacting Compound 4a with a reducing agent to produce Compound 5a;
reacting Compound 5a with p-toluenesulfonyl chloride to produce Compound 6;
reacting Compound 6a with a base to produce Compound 7a; and
reacting Compound 7a with an acid to produce Compound 1 acid salt (e.g., Compound 1 Tosylate, Compound 1 Besylate, Compound 1 Hydrochloride, Compound 1 Fumarate, or Compound 1 Phosphate).

In some embodiments, provided herein are compounds observed or isolated in any of the processes described, including the intermediate compounds observed or isolated in the preparation of Compound 1.

In some embodiments, provided herein is tert-butoxycarbonyldimethylamine (Compound 1a). In some embodiments, provide herein is lithiated tert-butoxycarbonyldimethylamine (Compound 2a).

In some embodiments, provided herein is 3,4-dihydro-1H-[1,4]oxazino[4,3-b]indazol-1-one (Compound 3a) or a salt thereof.

In some embodiments, provided herein is tert-butyl (2-(2-(2-hydroxyethyl)-2H-indazol-3-yl)-2-oxoethyl)(methyl)carbamate (Compound 4a) or a salt thereof.

In some embodiments, provided herein is tert-butyl (R)-(2-hydroxy-2-(2-(2-hydroxyethyl)-2H-indazol-3-yl)ethyl)(methyl)carbamate (Compound 5a). In some embodiments, provided herein is (R)-2-(3-(2-((tert-butoxycarbonyl)(methyl)amino)-1-hydroxyethyl)-2H-indazol-2-yl)ethyl 4-methylbenzenesulfonate (Compound 6a).

In some embodiments, provided herein is tert-butyl (R)-((3,4-dihydro-1H-[1,4]oxazino[4,3-b]indazol-1-yl)methyl)(methyl)carbamate (Compound 7a) or a salt thereof.

In some embodiments, provided herein is 1H-indazole-3-carboxylic acid (Compound 8a) or a salt thereof.

In some embodiments, provided herein is 2-bromoethyl 1H-indazole-3-carboxylate (Compound 9a) or a salt thereof.

### EXAMPLES

### Example 1

### Preparation of Salts of Compound 1

Preparation of Compound 1 salts was carried out by using ten different counter ions and six kinds of solvent. Each obtained salt was characterized by FT-Raman, XRPD, ¹H-NMR, thermal analysis and DVS to confirm the physical properties. The conditions and equipment used to characterize the products are provided below.

FT-Raman analyses were performed by using Bruker Optics MultiRAM. Each sample was measured. Excitation wavelength (1064 nm) was generated with a Nd:YAG laser (Laser Power: 500 mW; Wavenumber Resolution: 4 cm⁻¹; Sample Scan: 128 times).

Ambient temperature XRPD spectra were obtained by X'pert Pro MPD (Spectris). X-rays were generated with Cu Kα radiation at 45 kV and 40 mA. The powder samples were put on the reflection-free sample plate. The range angle was from 4 to 30 or 40° (2θ). Step size was 0.017° and time per step was 100 s. The XRPD patterns were analyzed by HighScorePlus.

Temperature controlled XRPD was measured by the X'pert-MPD with an Anton Paar TCU 100 Temperature Control Unit. Sample was measured from 30° C. to 250° C. or 270° C. with 10° C. increments. Measurement conditions were as follows: range: 4-30°, step: 0.017°, time per step: 100 s.

DSC chart was obtained using a TA Instruments DSC Q1000 differential scanning calorimeter. Each sample was analyzed in a crimped pan with a pinhole, heated under a nitrogen purge at a rate of 10° C./min, from 10° C. to 250° C. or 300° C.

TGA analyses were performed using a TA Instruments TGA Q500 thermogravimetric analyzer. Each sample was analyzed on a platinum pan, heated under a nitrogen purge at a rate of 10° C./min, from room temperature to 250° C. or 300° C.

DVS moisture sorption isotherms were generated using a Hiden Analytical IGA-sorp moisture sorption analyzer. Samples were run as received without pre-analysis drying. Each sample was equilibrated between about 30 and 120 minutes at each relative humidity (RH) step. Temperature was fixed at about 25° C. and the relative humidity steps (0 to 95% RH) were in 5-10% increments. Sample sizes ranged from 3 to 30 mg. After DVS measurement, XRPD was measured in order to check the solid form.

¹H-NMR spectra were obtained by AV400M Bruker BioSpin 400 MHz. D₂O was used for diluent. (Sample scan: 16 times).

The preparation of various salts was performed. Compound 1 (free base) and each of the inorganic and organic acids providing ten kinds of counter ion (HCl, benzensulfonic acid, citric acid, fumaric acid, H₃PO₄, L-malic acid, L-tartaric acid, MeSO₃H, p-toluenesulfonic acid, and succinic acid, those of which provide chloride, besylate citrate, fumarate, phosphate, L-malate, L-tartrate, mesylate, tosylate, and succinate anions, respectively) was dissolved in methanol. Each acid methanol solution was added into the Compound 1 methanol solution in a molar ratio of 1:1.5. These solutions were shaken with a shaking incubator at about 25° C. Methanol was then evaporated, and each solvent (methanol, AcOEt or THF) was added to each vial. After the solutions were shaken at about 40° C. for about 4 h, they were left at about 25° C. If crystallization did not occur, solvents were evaporated gradually by opening the cap of the vial. If no crystallization occurred, a second solvent (acetone, diisopropylether or toluene) was added to each vial. After the solutions were shaken at about 40° C. for about 4 h, they were left at about 25° C. If crystallization did not occur, solvents were evaporated gradually by opening the cap of the vial. Crystals obtained in the vials were measured directly by FT-Raman. Solids were then collected and evaluated by XRPD, thermal analysis, DVS and ¹H-NMR as needed.

The HCl, phosphate, fumarate, besylate and tosylate salts of Compound 1 were crystallized. From thermal analysis, phosphate has a large amount of weight loss from low temperature and XRPD spectra were a little different each other. ¹H-NMR spectrum of fumarate demonstrates that it does not always contain organic solvent, so the weight loss by TGA seemed to be comparable to the water dehydration. Therefore, this fumarate salt may be a hydrated form; and as a result of DVS, it has no hygroscopicity. Besylate has no weight loss until melting. Tosylate has a sharp XRPD spectrum.

Tosylate and besylate have less hygroscopicity and good thermal behavior. Note that sulfonic acid group generates the sulfonic ester by reacting with alcohol. Sulfonic ester is genotoxic impurity. Therefore, using alcohol should be avoided especially in the crystallized process.

To summarize this screening experiment:
(i) two fumarate salt polymorphs are obtained. Both polymorphs are hydrated form. Weight loss from low temperature was observed.
(ii) one besylate polymorph is obtained. This polymorph is an anhydrous form. Melting point is about 221° C. It has less hygroscopicity (about 2% weight gain at 95% RH).
(iii) one tosylate polymorph is obtained. This polymorph is an anhydrous form. Melting point is about 237° C. It has less hygroscopicity (about 1% weight gain at 95% RH).

### Example 2

### Preparation Study of Crystalline Salts of Compound 1

As a preparation study of crystalline salts of Compound 1, fumarate, besylate and tosylate salts were crystallized. The study of fumarate, besylate and tosylate was performed according to Table 2 and/or as described below. The conditions and equipment used to characterize the products are similar as those provided in Example 1.

TABLE 2
The summary result of preparation study of fumarate and besylate

| | | | Free | Acid | |
|---|---|---|---|---|---|
| | Acid | | base | weight | Crystal |
| Solvent | Counter ion | eq. | (mg) | (mg) | form |
| Acetone | Benzenesulfonic acid | 1 | 118 | 87 | BA |
| | monohydrate | | | | |
| | (Besylate) | | | | |
| THF | Fumaric acid | 1 | 118 | 56 | FB |
| | (Fumarate) | | | | |
| THF | Fumaric acid | 1 | 59 | 28 | FA |
| | (Fumarate) | | | | |
| THF/H₂O(15:1) | Fumaric acid | 1 | 59 | 28 | FA |
| mixture | (Fumarate) | | | | |
| Acetone | Benzenesulfonic acid | 1 | 118 | 87 | BA |
| | monohydrate | | | | |
| | (Besylate, 2^{nd} trial) | | | | |

### Compound 1 Besylate

About 118 mg of Compound 1 (free base) and 87 mg of benzenesulfonic acid monohydrate were added to a glass vial. 2 mL of acetone was poured into the glass vial and shaken by shaking incubator at about 60° C. for about 2 h. After it was stored at about RT all night, the solid obtained was filtered and dried under reduced pressure.

About 118 mg of Compound 1 (free base) and 87 mg of benzenesulfonic acid monohydrate were added to a glass vial. 2 mL of acetone was poured into the glass vial and shaken by shaking incubator at about RT for about 3 h. After that, the solid obtained was filtered and dried under reduced pressure. The resulting solid was suspended with 2 mL acetone at about RT for about 1 h. After that, it was filtered and dried under reduced pressure.

Thermal analysis indicates that besylate salt Form BA is anhydrous form and has positive thermal behavior. As a result of DVS, this form has less hygroscopicity. There is about 2% weight gain at 90% RH and about 8% weight gain at 95% RH with one batch of Form BA. There is about 1% weight gain at 90% RH and about 2% weight gain at 95% RH with another batch of Form BA. The material that was suspended in acetone twice has better quality than the material that did not. From temperature controlled XRPD, Form BA is not transformed to any other polymorph.

### Compound 1 Fumarate

About 118 mg of Compound 1 (free base) and about 56 mg of fumaric acid were added to a glass vial. About 2 mL of THF was poured into the glass vial and shaken by shaking incubator at about 60° C. for about 2 h. After it was stored at RT all night, the solid obtained was filtered and dried under reduced pressure.

About 59 mg of Compound 1 (free base) and about 28 mg of fumaric acid were added to a glass vial. About 2 mL of THF was poured into the glass vial and shaken by shaking incubator at RT for about 4 h. After that, the solid obtained was filtered and dried under reduced pressure.

About 59 mg of Compound 1 (free base) and about 28 mg of fumaric acid were added to the glass vial. About 2 mL of THF/H20 mixture (1:1) was poured into the glass vial and shaken by shaking incubator at RT for about 4 h. After that, the solid obtained was filtered and dried under reduced pressure.

Fumaric acid salt Form FB has about 3.8% weight loss from low temperature with TGA measurement. From DVS result, there is about 5% weight gain at over 75% RH, so polymorphic form seems to be changed. Note that when Form FB was placed under 75% RH overnight (created by a NaCl water suspension), Form FA was observed. Therefore, Form FA seems to be di-hydrate. Form FB seems to be mono-hydrate. The thermal behavior of Form FA is a little different from that of Form FB. Form FA generated the experiment described above using about 28 mg of fumaric acid and about 2 mL of THF seems to be easy to be dehydrated. It can be affected by vacuum dry. When Form FA and Form FB are placed under dry condition or heated, anhydrous form can be obtained. But they are quickly transformed to be hydrous form by obtaining water in the air.
Compound 1 Tosylate

10% aqueous sodium bicarbonate (about 25 mL) and chloroform (about 25 mL) was added to a solution of Compound 1 Hydrochloride Form HA (di-hydrochloride mono-hydrate of Compound 1; about 1000 mg, 3.21 mmol) in water (about 25 mL).After the separation, the aqueous layer was extracted with chloroform (about 25 mL). The combined organic layers were concentrated in vacuo. p-Toluenesulfonic acid monohydrate (about 648 mg, 1.05 g, TCI Lot No. FGL01) and methanol (about 10 mL) were added to the residual oil. The solution was concentrated in vacuo. The residual solid was suspended with acetone (about 20 mL) at RT for about 1 h. After that, it was filtered, washed with acetone (5 mL) and dried under the reduced pressure to afford a tosylate (about 1200 mg, 100%) as a white crystal.

Thermal analysis indicates that tosylate Form TA is an anhydrous form and has positive thermal behavior. As a result of DVS (isotherm not shown), this form has less hygroscopicity. There is about 1% weight gain at 95% RH. From temperature controlled XRPD, Form FA is not transformed to any other polymorph.

### Summary

Fumarate, besylate and tosylate salts were crystallized. From thermal analysis, the phosphate salt has a large amount of weight loss from low temperature and XRPD spectra were a little different each other. Two polymorphs were obtained for fumarate salt. Form FA was di-hydrate. Form FB was mono-hydrate. With regard to besylate and tosylate salts, one form has been obtained for each salt. Form BA and Form TA have good thermal behavior and good hygroscopicity behavior. From temperature controlled XRPD, Form BA and Form TA are not transformed to any other polymorph. Note that sulfonic acid generates the sulfonic ester by being reacted with alcohol.

### Example 3

### Preparation and Characterization of Compound 1 Hydrochloride

A study to evaluate Compound 1 Hydrochloride was carried out. The study included solvent screening, slurry screening and scale up study. In the case that polymorphic crystals were generated in the studies, their physical properties were determined in order to pursue a suitable crystal form of the salt form.

### Instrumental Techniques

FT-Raman analyses were performed by using MultiRAM (Bruker Optics). Each suspension or dry powder sample was measured from out of glass vial. The measurement condition is shown below:
Excitation wavelength: 1064 nm
Laser power: 500 mW
Wavenumber: 4 cm⁻¹
Sample scan: 128 times

X-ray Powder Diffraction (XRPD) spectra were obtained by X'pert Pro MPD (Spectris). X-rays was generated with Cu Kα radiation. The powder samples were put on the reflection free sample plate. The XRPD patterns were analyzed by HighScorePlus. The measurement condition is shown below:
Range angle: From 4 to 30°, to 40° and to 65° (2θ)
Step size: 0.017°
Time per step: 100 s

Differential Scanning calorimeter (DSC) chart was obtained by DSC Q1000 differential scanning calorimeter (TA Instruments). Each sample was analyzed in a crimped pan with a pinhole. The measurement condition is shown below:
Atmosphere: Nitrogen
Elevated temperature rate: 10° C./min
Temperature range: From 10° C. to 250 or 300° C.

Thermo-Gravimetric Analyzer (TGA) analyses were performed using TGA Q500 thermogravimetric analyzer (TA Instruments). Each sample was analyzed on a platinum pan. The measurement condition is shown below:
Atmosphere: Nitrogen
Elevated temperature rate: 10° C./min
Temperature range: From room temperature to 250 or 300° C.

Dynamic Vapor sorption (DVS) moisture sorption isotherms were generated using IGA-sorp moisture sorption analyzer (Hiden Analytical). Sample measurements were run without drying before analyses. XRPD was measured in order to check any changes of the solid form after the moisture sorption/desorption. The measurement condition is shown below:
Time at each RH steps: From 30 to 120 minutes
Relative humidity steps: 5 or 10% steps
Temperature: 25° C.

¹H-NMR spectra were obtained by AV400M (400 MHz, Bruker BioSpin). The measurement condition is shown below:
Diluent: D₂O
Sample Scans: 16 s

Elemental analysis: The amount of carbon, hydrogen and nitrogen element was measured by Flash 2000CHN elemental analyzer (Thermo Fisher Scientific, Inc.). The measurement condition is shown below:
Combustion furnace temperature: 1000° C.
Reduction furnace temperature: 700° C.
Column oven temperature: 50° C.
Analysis time: 600 s
Helium gas flow: 110 mL/min
Oxygen gas flow: 250 mL/min

Ion Chromatography: The amount of Chloride ion was measured using Chloride Ion Standard Solution (Cl⁻1000) by Ion Chromatography System ICS-5000+ (Thermo Fisher Scientific Inc.). The measurement condition is shown below;
Detection: Conductivity
Column: IonPac AG11-HC −0.4 μm (0.4 mm i.d.×50 mm, Thermo Fisher Scientific Inc.) and IonPac AS11 —HC-0.4 μm (0.4 mm i.d.>250 mm, Thermo Fisher Scientific Inc.)
Suppressor: ACES 300 Anion Capillary
Eluent: Potassium hydroxide eluent was prepared electronically using eluent generator ICS-5000+ EG equipped with potassium hydroxide. The concentration of potassium hydroxide eluent was changed from 1 mM to 30 mM by 44 min (1 mM/0-8 min, 1-10 mM/8-17 min, 10-15 mM/17-27 min, 15-30 mM/27-32 min, 30 mM/32-44 min).
Column temperature: 30° C.
Flow rate: 1.0 mL/min

### Solvent Screening for Crystallization

Compound 1 Hydrochloride, Form HA (about 5 mg) was put into each small glass vial and various types of solvents were added until the sample was dissolved at about 90° C. or boiling point. Maximum volume of solvent was about 500 μL. After the solution and suspension was heated for a few hours, the heater was switched off and the sample was cooled. The vials were preserved at about 5° C. Crystals obtained in the vials were measured directly by FT-Raman. After solvent was evaporated by opening the cap of vial, crystals obtained were also measured directly by FT-Raman. Solids were then collected and evaluated by XRPD and thermal analysis as needed.

### Slurry Screening by Using the Mixture of Organic Solvent and Water

Compound 1 Hydrochloride, Form HA (about 10 mg) was put into each small glass vial and various types of organic solvents mixed with water were added. After each sample was suspended at room temperature for about 3 days, suspended solids in the vials were measured directly by FT-Raman. Furthermore, each sample was suspended at about 50° C. for about 3 days and these were measured directly by FT-Raman. Solids were then collected and evaluated by XRPD, thermal analysis and DVS as needed.

### Scale up Study of Form HC

Compound 1 Hydrochloride, Form HA (about 100 mg) was put into a glass vial and about 1 mL of EtOH mixed with water (10:1) was added. After it was suspended at room temperature for 3 days, solid obtained was filtered and dried in vacuo at about 40° C. It was analyzed by XRPD, thermal analysis, ¹H-NMR, elemental analysis and ion-chromatography.

### Results

The experimental conditions and the brief results acquired from the solvent screening and the slurry screening by using the mixture of organic solvent and water are listed in Table 3A and Table 3B. From these screening studies, the four polymorphs, Form HA, Form HB, Form HC, and Form HD, were identified by FT-Raman spectroscopy and XRPD pattern.

TABLE 3A
The summary result of solvent screening
for Compound 1 Hydrochloride

| | FT-raman | FT-raman | |
|---|---|---|---|
| Solvent | (suspension) | (dry) | XRD |
| H₂O | | A | |
| MeOH | | A | |
| EtOH | | A | |
| ⁱPrOH | B | C | A + C |
| Chloroform | A | A | A |
| MeCN | B | A | |
| 1,2-Dimethoxyethane | A | A | |
| THF | A | A | |
| 1,4-Dioxane | A | A | |
| ^{t}Butyl Methyl Ether | A | A | |
| Diisopropyl Ether | A | A | |
| CPME | A | A | |
| AcOEt | A | A | |
| AcOⁱPr | A | A | |
| AcOⁱBu | A | A | |
| Acetone | B | A | |
| MEK | A | A | |
| MIBK | A | A | |
| Heptane | A | A | |
| Chlorobenzene | A | A | |
| Toluene | A | A | |
| Xylene | A | A | |
| MeOH—iPr₂O | B | A | |
| MeOH + water (20:1) | | A | |
| MeOH + water (5:1) | | A | |
| EtOH + water (20:1) | | A + C | A + C |
| EtOH + water (5:1) | | A | |
| IPA + water (20:1) | | D | C + D |
| IPA + water (5:1) | | A | |
| MeCN + water (20:1) | A | A | |
| MeCN + water (5:1) | | A | |
| Acetone + water (20:1) | A | A | |
| Acetone + water (5:1) | | A | |

TABLE 3B
The summary result of slurry screening
for Compound 1 Hydrochloride

| | FT-raman | FT-raman | | |
|---|---|---|---|---|
| Solvent | RT shaking | 50° C. shaking | XRD | XRD |
| EtOH + water (20:1) | A + C | A + C | | |
| EtOH + water (15:1) | C | A + C | | |
| EtOH + water (10:1) | C | — | C | C |
| IPA + water (20:1) | C | A + C | | |
| IPA + water (15:1) | C | C | | |
| IPA + water (10:1) | D | — | C + D | C |
| MeCN + water (20:1) | A | A | | |
| MeCN + water (15:1) | A | A | | |
| MeCN + water (10:1) | — | — | | |
| Acetone + water (20:1) | A | A | | |
| Acetone + water (15:1) | A | A | | |
| Acetone + water (10:1) | A | A | | |

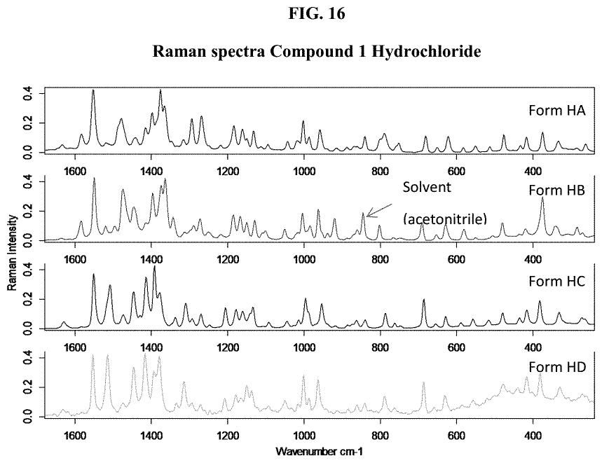

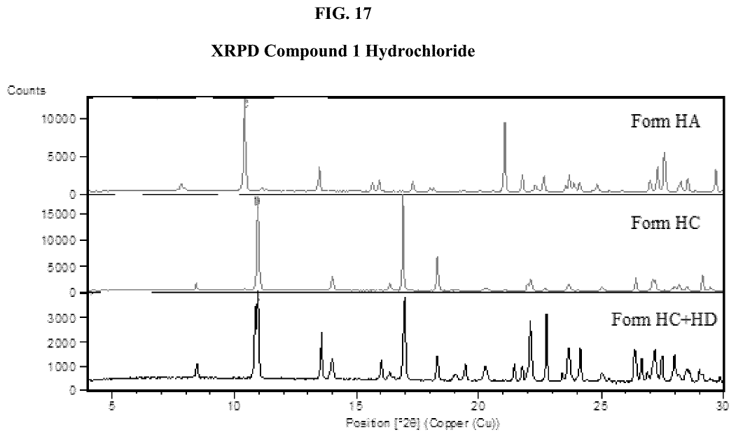

The Raman spectra and XRPD patterns of four polymorphs obtained with polymorph screening were shown in FIG. 16 and FIG. 17. The detailed information on the physical properties of Form HA, Form HB, Form HC, and Form HD are described below.

The XRPD pattern of Form HA is shown in FIG. 8, and XRPD peaks listing is shown in Table 3G. The thermal behavior of Form HA is shown in FIG. 10. The TG curve showed over 17.1% weight loss from 70° C., suggesting that the material was solvated and HCl was eliminated by heating. The NMR spectra indicated no presence of organic solvents. These data suggested that Form HA was a hydrated form. The DVS curve of Form HA is provided in FIG. 11. The data indicated that Form HA was highly hygroscopic because over 100% weight gain was seen at the humidity level of 95% RH. Some portion of the material might be dissolved under a high-humidity environment. The XRPD patterns from the samples before and after the DVS measurement (moisture absorption/desorption, data not shown) indicated that Form HA was transformed to Form HC mixed with an unidentified polymorph. ¹H-NMR spectra of hydrochloric acid salt Form HA and Form HC in D₂O(data not shown) were quite similar, except for the chemical shifts around at 7.7 ppm and 4.5 ppm. The results of the elemental analysis including the ion-chromatography show that the elements of Form HA corresponded to be di-HCl salt and mono-H₂O. The slight difference observed in the NMR spectra might be caused by the difference of numbers of HCl salt, i.e., Form HA was di-HCl and Form C was mono-HCl.

Form HB was observed only in a suspension state and transformed to Form HC or Form HA when it was filtrated and dried. In order to identify Form HB, FT-Raman was available because it can generally work for samples in a suspension state (see FIG. 16). The XRPD pattern could not be acquired.

It was confirmed that Form HC was reproducible even in the scale up study. The XRPD pattern of Form HC is shown in FIG. 12 and XRPD peak listing is shown in Table 3H. The thermal behavior of Form HC is provided in FIG. 14. The TG curve showed 6.6% weight loss from 70° C., suggesting that the material was solvated. The NMR spectrum shown indicated no presence of organic solvents. These results suggested that Form HC was a hydrated form. The DVS curve of Form HC is provided in FIG. 15, which indicated that Form HC was hygroscopic because 9.3% weight gain was observed when the humidity was increased from 0% RH to 95% RH. From the XRPD patterns of the samples before and after DVS measurement (moisture sorption/desorption), it was found that Form HC was basically stable. However, the Raman spectra indicated that Form HC transformed to Form HD when it was stored at a high-humid condition (stored at a humidity of 75% RH for 1 day by using a glass vial with saturated sodium chloride water). The results of the elemental analysis show that Form HC corresponded to be mono-HCl salt mono-hydrate.

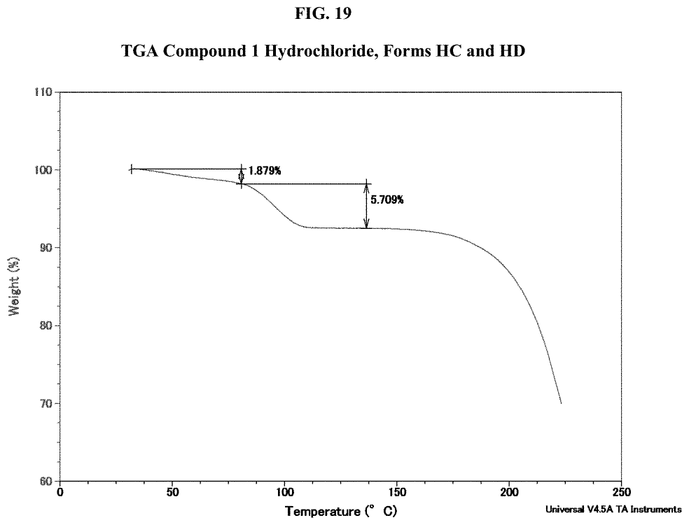

The thermal behavior of Form C+D is provided in FIG. 19. The TG curve showed 7.6% weight loss from 30° C., suggesting that the material was solvated. Form HD was found to correspond to be mono-HCl di-hydrate because Form HC was transformed to Form HD at the high humidity and the weight gain (between 60% RH and 70% RH), 6.5%, approximately corresponded to the equivalent amount of one H₂O molecule of Compound 1 mono-HCl, 6.6%.

### Summary

The polymorph study including solvent screening, slurry screening and scale up study was performed. As the result, the following four polymorphs were obtained:
Form HA: Di-HCl salt mono-hydrate. Highly hygroscopic.
Form HB: Not stable because it was obtained only in a suspension state.
Form HC: Mono-HCl salt mono-hydrate. Transformed to Form D at the humidity level of more than 70% RH.
Form HD: Mono-HCl salt di-hydrate. Transformed to Form C at room temperature.

TABLE 3C
Form HA

| Pos. | Intensity | FWHM Left | d-value | Relative intensity |
|---|---|---|---|---|
| [°2Th.] | [cts] | [°2Th.] | [Å] | [%] |
| 7.8 | 1004 | 0.082 | 11.29 | 8.1 |
| 8.0 | 419 | 0.082 | 11.09 | 3.4 |
| 10.4 | 12395 | 0.082 | 8.48 | 100.0 |
| 11.1 | 420 | 0.082 | 7.94 | 3.4 |
| 13.5 | 3173 | 0.082 | 6.57 | 25.6 |
| 14.0 | 119 | 0.122 | 6.30 | 1.0 |
| 14.8 | 104 | 0.122 | 5.97 | 0.8 |
| 15.6 | 1197 | 0.061 | 5.66 | 9.7 |
| 15.9 | 1575 | 0.061 | 5.56 | 12.7 |
| 17.3 | 1277 | 0.082 | 5.12 | 10.3 |
| 18.0 | 390 | 0.061 | 4.92 | 3.2 |
| 18.2 | 388 | 0.061 | 4.88 | 3.1 |
| 19.3 | 74 | 0.245 | 4.59 | 0.6 |
| 20.0 | 170 | 0.082 | 4.43 | 1.4 |
| 20.6 | 116 | 0.122 | 4.31 | 0.9 |
| 21.1 | 9312 | 0.102 | 4.22 | 75.1 |
| 21.8 | 2297 | 0.082 | 4.08 | 18.5 |
| 22.3 | 941 | 0.102 | 3.98 | 7.6 |
| 22.7 | 2000 | 0.102 | 3.92 | 16.1 |
| 23.5 | 846 | 0.061 | 3.78 | 6.8 |
| 23.7 | 2190 | 0.082 | 3.75 | 17.7 |
| 23.9 | 1213 | 0.102 | 3.72 | 9.8 |
| 24.1 | 1224 | 0.061 | 3.69 | 9.9 |
| 24.8 | 993 | 0.082 | 3.58 | 8.0 |
| 25.3 | 184 | 0.082 | 3.51 | 1.5 |
| 25.8 | 259 | 0.082 | 3.45 | 2.1 |
| 26.4 | 87 | 0.122 | 3.37 | 0.7 |
| 27.0 | 1637 | 0.082 | 3.30 | 13.2 |
| 27.3 | 3326 | 0.082 | 3.26 | 26.8 |
| 27.6 | 5175 | 0.102 | 3.23 | 41.8 |
| 28.3 | 1379 | 0.082 | 3.15 | 11.1 |
| 28.5 | 1704 | 0.082 | 3.13 | 13.8 |
| 28.8 | 136 | 0.082 | 3.09 | 1.1 |
| 29.7 | 3019 | 0.082 | 3.01 | 24.4 |
| 30.1 | 2316 | 0.082 | 2.97 | 18.7 |
| 30.7 | 95 | 0.122 | 2.91 | 0.8 |
| 31.2 | 2196 | 0.082 | 2.86 | 17.7 |
| 31.6 | 1138 | 0.082 | 2.83 | 9.2 |
| 32.1 | 1042 | 0.082 | 2.78 | 8.4 |
| 32.9 | 758 | 0.082 | 2.72 | 6.1 |
| 33.5 | 389 | 0.082 | 2.67 | 3.1 |
| 33.8 | 355 | 0.102 | 2.65 | 2.9 |
| 34.0 | 586 | 0.082 | 2.63 | 4.7 |
| 34.8 | 458 | 0.082 | 2.58 | 3.7 |
| 35.8 | 107 | 0.122 | 2.51 | 0.9 |
| 36.1 | 352 | 0.061 | 2.49 | 2.8 |
| 36.4 | 472 | 0.102 | 2.47 | 3.8 |
| 36.9 | 285 | 0.102 | 2.43 | 2.3 |
| 37.8 | 818 | 0.082 | 2.38 | 6.6 |
| 38.2 | 382 | 0.061 | 2.35 | 3.1 |
| 38.8 | 497 | 0.061 | 2.32 | 4.0 |
| 39.4 | 314 | 0.061 | 2.28 | 2.5 |
| 39.7 | 219 | 0.082 | 2.27 | 1.8 |

TABLE 3D
Form HC

| Pos. | Intensity | FWHM Left | d-value | Relative intensity |
|---|---|---|---|---|
| [°2Th.] | [cts] | [°2Th.] | [Å] | [%] |
| 8.4 | 1196 | 0.061 | 10.47 | 6.5 |
| 10.4 | 240 | 0.082 | 8.48 | 1.3 |
| 11.0 | 17819 | 0.082 | 8.07 | 96.9 |
| 14.0 | 2618 | 0.102 | 6.32 | 14.2 |
| 16.4 | 1401 | 0.102 | 5.42 | 7.6 |
| 16.9 | 18387 | 0.082 | 5.25 | 100.0 |
| 18.3 | 6429 | 0.082 | 4.85 | 35.0 |
| 19.1 | 166 | 0.143 | 4.65 | 0.9 |
| 20.3 | 445 | 0.143 | 4.38 | 2.4 |
| 21.0 | 156 | 0.163 | 4.22 | 0.9 |
| 22.0 | 1179 | 0.061 | 4.04 | 6.4 |
| 22.1 | 2062 | 0.102 | 4.02 | 11.2 |
| 22.7 | 490 | 0.061 | 3.91 | 2.7 |
| 23.7 | 1324 | 0.143 | 3.76 | 7.2 |
| 24.0 | 135 | 0.163 | 3.70 | 0.7 |
| 25.0 | 751 | 0.082 | 3.55 | 4.1 |
| 26.4 | 2469 | 0.082 | 3.37 | 13.4 |
| 27.1 | 2279 | 0.082 | 3.29 | 12.4 |
| 27.2 | 1835 | 0.082 | 3.27 | 10.0 |
| 28.0 | 798 | 0.061 | 3.18 | 4.3 |
| 28.2 | 1311 | 0.082 | 3.16 | 7.1 |
| 28.5 | 660 | 0.143 | 3.13 | 3.6 |
| 29.1 | 2970 | 0.082 | 3.06 | 16.2 |
| 29.5 | 714 | 0.082 | 3.03 | 3.9 |

### Example 4

### Preparation and Characterization of Compound 1 Tosylate

Compound 1 Tosylate can be prepared according to the conditions described in Example 1 and Example 2. Further, a polymorph study of toluenesulfonic acid salt was carried out. The study included solvent screening, rapid cool screening and slurry screening. In the case that polymorphic crystals were generated in the studies, their physical properties were determined in order to pursue a suitable crystal form of the salt form.

### Instrumental Techniques

FT-Raman, XRPD, DSC, TGA, and DVS instruments and techniques are similar to those in Example 3. Temperature controlled XRPD was measured by X'pert-Pro MPD (Spectris) with TCU 100 Temperature Control Unit (Anton Paar). The temperature of the sampling stage was increased step by step as programmed and XRPD patterns of sample were measured at each step. The measurement condition is shown below:
Range angle: From 4 to 30°
Step size: 0.017°
Time per step: 100 s

### Solvent Screening

Form TA (5 mg) was put into each small glass vial and various types of solvents were added until the sample was dissolved at 90° C. or boiling point. Maximum volume of solvent was 500 μL. After the solution and suspension was heated for a few hours, the heater was switched off and the sample was cooled slowly. The vials were preserved at 5° C. Crystals obtained in the vials were measured directly by FT-Raman. After solvent was evaporated by opening the cap of vial, crystals obtained were also measured directly by FT-Raman. Solids were then collected and evaluated by XRPD as needed.

Solvents: water, MeOH, EtOH, iPrOH, chloroform, MeCN, 1,2-dimethoxyethane, THF, 1,4-dioxane, tbutyl methyl ether, diisopropyl ether, CPME, AcOEt, AcOiPr, AcOiBu, acetone, MEK, heptane, chlorobenzene, toluene, xylene, MeOH/iPr₂O (1:2), EtOH/iPr₂O (8:7), EtOH/Heptane (1:1), NMP/iPr₂O (2:1), IPA/H₂O(20:1), IPA/H₂O(10:1), and MeCN/H₂O(10:1) generated Form TA.

### Rapid Cool Screening

Form TA (5 mg) was dissolved at 90° C. or boiling point. After a few hours, the solution was cooled at ice bath. After that, the vials were preserved at −20° C. for 1 day. Crystals obtained in the vials were measured directly by FT-Raman. After solvent was evaporated by opening the cap of vial, crystals obtained were also measured directly by FT-Raman. Solids were then collected and evaluated by XRPD as needed.

Solvents: MeOH, EtOH, iPrOH, chloroform, acetone/H₂O (20:1), acetone/H₂O (10:1), THF/H₂O (20:1), MeOH/iPr₂O (1:1), EtOH/iPr₂O (2:1), and EtOH/heptane (2:1) generated Form TA.

### Slurry Screening

Form TA (10 mg) was suspended in selected solvent systems at room temperature and 50° C. and shaken for 14 days. Crystals obtained in the vials were measured directly by FT-Raman. After solvent was evaporated by opening the cap of vial, crystals obtained were also measured directly by FT-Raman. The collected solids were analyzed by XRPD as needed.

At room temperature, solvents: iPrOH, MeCN, THF, tbutyl methyl ether, AcOEt, acetone, toluene, IPA/H₂O (10:1), THF/H₂O (10:1), acetone/H₂O (10:1), and MeCN/H₂O (10:1) generated Form TA. At 50° C., solvents iPrOH, MeCN, THF, tbutyl methyl ether, AcOEt, acetone, toluene, IPA/H₂O (10:1), THF/H₂O (10:1), acetone/H₂O (10:1), and MeCN/H₂O (10:1) generated Form TA.

The representative XRPD pattern of the single crystal form, Form TA, generated in the screens is provided in FIG. 1, and listing of peaks is shown in Table 4. The thermal behavior of this material is shown in FIG. 2. The DSC curve showed endothermic peak from 236° C., which was attributed to melting. TG curve indicated no weight loss until melting, indicating that this material was not solvated. The DVS curve is provided in FIG. 4, which showed that the crystal form approximately gained 1% weight of the material according to the stepwise increase of the humidity from 0% RH up to 95% RH. XRPD patterns indicated that no changes of the crystal form were occurred before and after the DVS measurement (data not shown). Temperature controlled XRPD (data not shown) indicated that Form TA was stable in terms of the crystal form and not transformed to another form until the melting point.

### Summary

The polymorph study including solvent screening, rapid cool screening and slurry screening was performed. As the result, Form TA was obtained as an anhydrous form with non-hygroscopicity. Melting point was about 236° C.

TABLE 4
Form TA

| Pos. | Intensity | FWHM Left | d-value | Relative intensity |
|---|---|---|---|---|
| [°2Th.] | [cts] | [°2Th.] | [Å] | [%] |
| 5.7 | 58798 | 0.102 | 15.62 | 100.0 |
| 8.8 | 204 | 0.122 | 10.02 | 0.4 |
| 11.3 | 31218 | 0.102 | 7.84 | 53.1 |
| 11.9 | 392 | 0.102 | 7.43 | 0.7 |
| 14.0 | 625 | 0.082 | 6.30 | 1.1 |
| 15.6 | 213 | 0.122 | 5.69 | 0.4 |
| 16.4 | 5924 | 0.102 | 5.40 | 10.1 |
| 16.7 | 1350 | 0.102 | 5.31 | 2.3 |
| 16.9 | 13331 | 0.082 | 5.23 | 22.7 |
| 17.6 | 342 | 0.102 | 5.02 | 0.6 |
| 18.3 | 2846 | 0.122 | 4.84 | 4.8 |
| 18.7 | 503 | 0.082 | 4.73 | 0.9 |
| 18.9 | 1238 | 0.082 | 4.69 | 2.1 |
| 19.4 | 1220 | 0.122 | 4.57 | 2.1 |
| 20.2 | 695 | 0.122 | 4.40 | 1.2 |
| 20.8 | 936 | 0.122 | 4.27 | 1.6 |
| 21.5 | 2320 | 0.082 | 4.13 | 4.0 |
| 21.9 | 463 | 0.102 | 4.05 | 0.8 |
| 22.5 | 1460 | 0.082 | 3.95 | 2.5 |
| 22.6 | 9670 | 0.082 | 3.92 | 16.5 |
| 22.9 | 571 | 0.122 | 3.88 | 1.0 |
| 23.4 | 772 | 0.102 | 3.80 | 1.3 |
| 23.9 | 489 | 0.082 | 3.73 | 0.8 |
| 24.2 | 1293 | 0.143 | 3.67 | 2.2 |
| 25.1 | 2106 | 0.122 | 3.55 | 3.6 |
| 25.8 | 262 | 0.122 | 3.45 | 0.5 |
| 26.5 | 934 | 0.082 | 3.36 | 1.6 |
| 26.9 | 2998 | 0.102 | 3.31 | 5.1 |
| 27.4 | 583 | 0.102 | 3.25 | 1.0 |
| 28.4 | 10582 | 0.082 | 3.14 | 18.0 |
| 28.7 | 2214 | 0.143 | 3.11 | 3.8 |
| 29.6 | 493 | 0.082 | 3.01 | 0.8 |
| 30.3 | 329 | 0.082 | 2.95 | 0.6 |
| 30.8 | 217 | 0.163 | 2.90 | 0.4 |
| 32.4 | 195 | 0.102 | 2.76 | 0.3 |
| 33.1 | 367 | 0.102 | 2.70 | 0.6 |
| 33.5 | 356 | 0.082 | 2.67 | 0.6 |
| 34.2 | 2950 | 0.082 | 2.62 | 5.0 |
| 35.3 | 838 | 0.102 | 2.54 | 1.4 |
| 35.8 | 165 | 0.122 | 2.51 | 0.3 |
| 36.1 | 240 | 0.163 | 2.49 | 0.4 |
| 36.7 | 174 | 0.082 | 2.45 | 0.3 |
| 37.7 | 151 | 0.122 | 2.38 | 0.3 |
| 38.3 | 459 | 0.122 | 2.35 | 0.8 |
| 39.4 | 212 | 0.143 | 2.29 | 0.4 |

### Example 5

### Preparation and Characterization of Compound 1 Besylate

Compound 1 Besylate, Form BA was prepared in accordance with the procedures set forth in Example 1 and Example 2. The conditions and equipment used to characterize the products are similar as those provided in Example 1.

Form BA was characterized by XRPD. The XRPD pattern is shown in FIG. 5 and the XRPD data are provided in the table below.

TABLE 5
Form BA

| Pos. | Intensity | FWHM Left | d-value | Relative intensity |
|---|---|---|---|---|
| [°2Th.] | [cts] | [°2Th.] | [Å] | [%] |
| 5.9 | 7619 | 0.082 | 14.89 | 90.2 |
| 9.0 | 619 | 0.082 | 9.81 | 7.3 |
| 10.4 | 168 | 0.082 | 8.53 | 2.0 |
| 11.8 | 5492 | 0.082 | 7.46 | 65.0 |
| 12.3 | 1428 | 0.082 | 7.19 | 16.9 |
| 15.3 | 615 | 0.082 | 5.80 | 7.3 |
| 16.1 | 349 | 0.061 | 5.50 | 4.1 |
| 17.1 | 4506 | 0.082 | 5.18 | 53.3 |
| 17.3 | 4902 | 0.082 | 5.13 | 58.0 |
| 17.5 | 1113 | 0.102 | 5.07 | 13.2 |
| 17.8 | 1054 | 0.082 | 4.98 | 12.5 |
| 18.2 | 8451 | 0.102 | 4.86 | 100.0 |
| 19.2 | 3173 | 0.163 | 4.62 | 37.6 |
| 19.4 | 2888 | 0.102 | 4.57 | 34.2 |
| 20.8 | 1593 | 0.082 | 4.27 | 18.9 |
| 21.0 | 864 | 0.082 | 4.24 | 10.2 |
| 22.5 | 1644 | 0.082 | 3.95 | 19.5 |
| 22.8 | 3918 | 0.122 | 3.90 | 46.4 |
| 22.9 | 2106 | 0.061 | 3.88 | 24.9 |
| 23.5 | 1015 | 0.082 | 3.78 | 12.0 |
| 23.8 | 3586 | 0.082 | 3.74 | 42.4 |
| 24.4 | 2809 | 0.082 | 3.64 | 33.2 |
| 24.7 | 925 | 0.061 | 3.60 | 11.0 |
| 24.9 | 760 | 0.082 | 3.58 | 9.0 |
| 25.7 | 4527 | 0.102 | 3.46 | 53.6 |
| 26.3 | 1953 | 0.102 | 3.38 | 23.1 |

Form BA was characterized by DSC (FIG. 6) and DVS (FIG. 7). The DSC thermogram revealed an endothermic event at an onset temperature of 224.0° C. with a peak temperature of 225.3° C.

### Example 6

### Preparation and Characterization of Compound 1 Fumarate

Compound 1 Fumarate, Form FA and FB were prepared in accordance with the procedures set forth in Example 1 and Example 2. The conditions and equipment used to characterize the products are similar as those provided in Example 1.

Form FA was characterized by XRPD. The XRPD pattern is shown in FIG. 20 and the XRPD data are provided in the table below.

TABLE 6A
Form FA

| Pos. | Intensity | FWHM Left | d-value | Relative intensity |
|---|---|---|---|---|
| [°2Th.] | [cts] | [°2Th.] | [Å] | [%] |
| 9.9 | 6724 | 0.102 | 8.92 | 100.0 |
| 11.4 | 687 | 0.082 | 7.76 | 10.2 |
| 12.0 | 801 | 0.143 | 7.39 | 11.9 |
| 14.8 | 2293 | 0.082 | 5.99 | 34.1 |
| 15.5 | 499 | 0.082 | 5.71 | 7.4 |
| 15.8 | 877 | 0.102 | 5.60 | 13.0 |
| 16.3 | 361 | 0.102 | 5.44 | 5.4 |
| 17.2 | 831 | 0.082 | 5.14 | 12.4 |
| 18.7 | 433 | 0.082 | 4.75 | 6.4 |
| 19.3 | 1184 | 0.102 | 4.61 | 17.6 |
| 19.9 | 4810 | 0.102 | 4.47 | 71.5 |
| 20.5 | 169 | 0.163 | 4.34 | 2.5 |
| 21.4 | 620 | 0.082 | 4.14 | 9.2 |
| 21.7 | 1794 | 0.082 | 4.09 | 26.7 |
| 22.6 | 212 | 0.163 | 3.94 | 3.2 |
| 22.9 | 434 | 0.082 | 3.89 | 6.5 |
| 23.3 | 1366 | 0.061 | 3.81 | 20.3 |
| 23.8 | 1619 | 0.082 | 3.73 | 24.1 |
| 24.0 | 3254 | 0.102 | 3.70 | 48.4 |
| 24.2 | 1047 | 0.061 | 3.68 | 15.6 |
| 24.9 | 1483 | 0.082 | 3.57 | 22.1 |
| 25.1 | 694 | 0.061 | 3.55 | 10.3 |
| 25.7 | 2944 | 0.102 | 3.46 | 43.8 |
| 26.4 | 365 | 0.102 | 3.37 | 5.4 |
| 26.8 | 1129 | 0.102 | 3.33 | 16.8 |
| 27.7 | 2825 | 0.102 | 3.22 | 42.0 |
| 28.2 | 564 | 0.102 | 3.17 | 8.4 |
| 28.4 | 191 | 0.082 | 3.14 | 2.8 |
| 28.8 | 335 | 0.082 | 3.10 | 5.0 |
| 28.9 | 418 | 0.082 | 3.08 | 6.2 |
| 29.8 | 1251 | 0.082 | 3.00 | 18.6 |

Form FA was characterized by DSC (FIG. 21), TGA (FIG. 22), and DVS (FIG. 23). The DSC thermogram revealed endothermic events at an onset temperature of 90.6° C. with a peak temperature of 100.9° C., and at an onset temperature of 146.6° C. with a peak temperature of 152.5° C.

Form FB was characterized by XRPD. The XRPD pattern is shown in FIG. 24 and the XRPD data are provided in the table below.

TABLE 6B
Form FB

| Pos. | Intensity | FWHM Left | d-value | Relative intensity |
|---|---|---|---|---|
| [°2Th.] | [cts] | [°2Th.] | [Å] | [%] |
| 8.8 | 17286 | 0.082 | 10.00 | 100.0 |
| 9.1 | 1848 | 0.102 | 9.67 | 10.7 |
| 11.9 | 380 | 0.082 | 7.43 | 2.2 |
| 12.4 | 260 | 0.122 | 7.12 | 1.5 |
| 14.6 | 2705 | 0.122 | 6.07 | 15.7 |
| 14.8 | 2644 | 0.122 | 5.99 | 15.3 |
| 15.5 | 377 | 0.082 | 5.72 | 2.2 |
| 16.0 | 933 | 0.122 | 5.53 | 5.4 |
| 16.6 | 1595 | 0.122 | 5.33 | 9.2 |
| 17.7 | 3977 | 0.102 | 5.02 | 23.0 |
| 18.3 | 587 | 0.122 | 4.85 | 3.4 |
| 19.4 | 2547 | 0.184 | 4.58 | 14.7 |
| 19.8 | 1061 | 0.143 | 4.48 | 6.1 |
| 20.3 | 4027 | 0.122 | 4.37 | 23.3 |
| 20.8 | 3367 | 0.122 | 4.27 | 19.5 |
| 21.2 | 1552 | 0.163 | 4.19 | 9.0 |
| 21.7 | 3763 | 0.122 | 4.09 | 21.8 |
| 22.7 | 1409 | 0.122 | 3.92 | 8.2 |
| 23.3 | 312 | 0.163 | 3.82 | 1.8 |
| 23.7 | 2195 | 0.122 | 3.76 | 12.7 |
| 23.9 | 2758 | 0.102 | 3.72 | 16.0 |
| 24.6 | 6099 | 0.184 | 3.62 | 35.3 |
| 25.3 | 489 | 0.061 | 3.52 | 2.8 |
| 26.3 | 826 | 0.163 | 3.38 | 4.8 |
| 26.6 | 610 | 0.082 | 3.35 | 3.5 |
| 27.4 | 291 | 0.082 | 3.25 | 1.7 |
| 27.6 | 792 | 0.102 | 3.23 | 4.6 |
| 27.8 | 738 | 0.061 | 3.20 | 4.3 |
| 28.0 | 536 | 0.102 | 3.18 | 3.1 |
| 28.3 | 911 | 0.122 | 3.15 | 5.3 |
| 29.3 | 576 | 0.143 | 3.04 | 3.3 |
| 29.8 | 1169 | 0.082 | 2.99 | 6.8 |

Form FB was characterized by DSC (FIG. 25), TGA (FIG. 26), and DVS (FIG. 27). The DSC thermogram revealed an endothermic event at an onset temperature of 156.1° C. with a peak temperature of 158.5° C.

### Example 7

### Preparation and Characterization of Compound 1 Phosphate

Compound 1 Phosphate, Form PA was prepared in accordance with the procedures set forth in Example 1. The conditions and equipment used to characterize the products are similar as those provided in Example 1.

Form PA was characterized by XRPD. The XRPD pattern is shown in FIG. 28 and the XRPD data are provided in the table below.

TABLE 7
Form PA

| Pos. | Intensity | FWHM Left | d-value | Relative intensity |
|---|---|---|---|---|
| [°2Th.] | [cts] | [°2Th.] | [Å] | [%] |
| 4.8 | 455 | 0.143 | 18.57 | 11.1 |
| 6.6 | 234 | 0.122 | 13.29 | 5.7 |
| 7.1 | 806 | 0.122 | 12.38 | 19.6 |
| 8.4 | 1440 | 0.163 | 10.57 | 35.0 |
| 9.0 | 630 | 0.122 | 9.80 | 15.3 |
| 9.4 | 661 | 0.122 | 9.40 | 16.1 |
| 10.0 | 626 | 0.122 | 8.80 | 15.2 |
| 10.3 | 639 | 0.122 | 8.56 | 15.5 |
| 10.6 | 678 | 0.122 | 8.37 | 16.5 |
| 11.1 | 1778 | 0.143 | 7.99 | 43.2 |
| 12.3 | 312 | 0.143 | 7.20 | 7.6 |
| 13.2 | 1044 | 0.143 | 6.69 | 25.4 |
| 13.6 | 1597 | 0.122 | 6.49 | 38.8 |
| 14.2 | 1784 | 0.082 | 6.22 | 43.3 |
| 14.5 | 3178 | 0.102 | 6.10 | 77.2 |
| 14.6 | 2660 | 0.082 | 6.06 | 64.6 |
| 15.0 | 750 | 0.122 | 5.90 | 18.2 |
| 15.4 | 1692 | 0.143 | 5.75 | 41.1 |
| 16.1 | 1992 | 0.143 | 5.51 | 48.4 |
| 16.3 | 344 | 0.102 | 5.42 | 8.4 |
| 16.8 | 167 | 0.143 | 5.29 | 4.1 |
| 17.2 | 138 | 0.245 | 5.16 | 3.4 |
| 17.9 | 206 | 0.122 | 4.95 | 5.0 |
| 18.3 | 2937 | 0.102 | 4.85 | 71.3 |
| 18.8 | 1013 | 0.122 | 4.71 | 24.6 |
| 19.2 | 979 | 0.082 | 4.63 | 23.8 |
| 19.8 | 617 | 0.082 | 4.48 | 15.0 |
| 20.1 | 615 | 0.082 | 4.42 | 14.9 |
| 20.3 | 4117 | 0.102 | 4.36 | 100.0 |
| 20.7 | 488 | 0.122 | 4.29 | 11.9 |
| 21.0 | 661 | 0.082 | 4.24 | 16.1 |
| 21.7 | 1486 | 0.082 | 4.09 | 36.1 |
| 22.1 | 1333 | 0.082 | 4.03 | 32.4 |
| 23.2 | 1415 | 0.163 | 3.83 | 34.4 |
| 23.6 | 3206 | 0.122 | 3.77 | 77.9 |
| 24.3 | 2015 | 0.122 | 3.66 | 48.9 |
| 24.6 | 1617 | 0.082 | 3.62 | 39.3 |
| 24.8 | 1231 | 0.122 | 3.59 | 29.9 |
| 25.1 | 3546 | 0.122 | 3.55 | 86.1 |
| 25.6 | 558 | 0.102 | 3.48 | 13.6 |
| 25.7 | 1035 | 0.122 | 3.46 | 25.1 |
| 26.0 | 1092 | 0.102 | 3.42 | 26.5 |
| 26.3 | 1298 | 0.102 | 3.39 | 31.5 |
| 26.6 | 691 | 0.082 | 3.35 | 16.8 |
| 26.8 | 699 | 0.082 | 3.32 | 17.0 |
| 27.2 | 573 | 0.163 | 3.28 | 13.9 |
| 27.5 | 738 | 0.102 | 3.25 | 17.9 |
| 27.7 | 721 | 0.122 | 3.22 | 17.5 |
| 28.2 | 485 | 0.082 | 3.17 | 11.8 |
| 28.4 | 397 | 0.122 | 3.14 | 9.6 |
| 28.7 | 939 | 0.102 | 3.11 | 22.8 |
| 29.0 | 580 | 0.082 | 3.08 | 14.1 |
| 29.4 | 465 | 0.204 | 3.04 | 11.3 |

Form PA was characterized by DSC (FIG. 29) and TGA (FIG. 30). The DSC thermogram revealed endothermic events at an onset temperature of 73.9° C. with a peak temperature of 88.4° C., and at an onset temperature of 168.2° C. with a peak temperature of 190.8° C.

### Example 8

### Preparation of Compound 1

### Preparation of tent-butyl(2-(2-(2-hydroxyethyl)-2H-indazol-3-yl)-2-oxoethyl)(methyl)carbamate (Compound 4a)

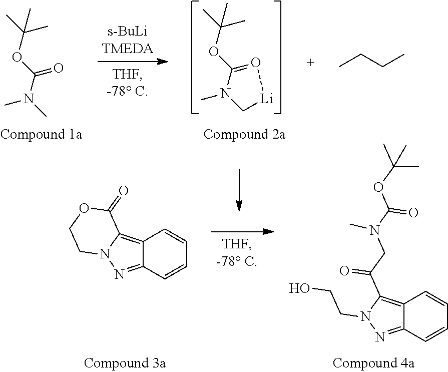

Charged 20.41 g of tert-butoxycarbonyldimethylamine (Compound 1a), 33.0 g of N,N,N′,N′-tetramethylethylenediamine (TMEDA) and 143.8 g of anhydrous tetrahydrofuran (THF) into 0.5 L round bottom flask (reactor A) equipped with a magnetic stirrer, temperature probe, and a nitrogen blanket. Adjusted the temperature of the reactor A to −75° C. and slowly charged 100 mL of sec-butyl lithium, [1.4M in cyclohexane] to reactor A via cannula keeping reaction temperature <−60° C. Stirred the reaction mixture at −70° C. for 1 hour.

Slowly charged the lithiated tert-butoxycarbonyldimethylamine (Compound 2a) solution (reactor A) to a mixture of 20.33 g of 3,4-dihydro-1H-[1,4]oxazino[4,3-b]indazol-1-one (Compound 3a) in 156.3 g of anhydrous THF while maintaining the temperature of the reaction mixture below −65° C. Agitated the reaction mixture at approximately −70° C. for 30 minutes. Slowly charged 9.7 g of acetic acid in 9.6 g of tetrahydrofuran (anhydrous not required) maintaining reaction temperature below −50° C. Adjusted the temperature to 0-5° C. and charged 55.9 g of solution of concentrated HCl in 215.3 g of water. Vacuum distilled the solution to a volume of 345 mL. Adjusted the reaction temperature to approximately 25° C. and charged 540.6 g of cyclopentyl methyl ether (CPME). Heated the reaction mixture to approximately 70° C. Agitated the mixture for 10 minutes at 70° C. then allowed the phases to separate and removed the aqueous layer. Washed the organic layer with 60 g of water twice. Vacuum distilled the mixture to a final volume of 214 mL then removed the vacuum. Heated the mixture to 95° C. to generate a solution and then cooled the solution at 1° C./min until the product precipitated. Aged the resulting slurry at the temperature of self-nucleation for 30 minutes then cooled the reaction mixture to 0° C. over 4 hours. Stirred the slurry at 0° C. for 30 minutes. Filtered the reaction mixture and washed the cake with 40 g of cold cyclopentyl methyl ether twice. The solids were dried under vacuum (>28 in.-Hg) at 50° C. to yield 26.8 g of tert-butyl (2-(2-(2-hydroxyethyl)-2H-indazol-3-yl)-2-oxoethyl)(methyl)carbamate (Compound 4a). ¹H NMR (400 MHz, CHLOROFORM-d) δ ppm 1.38 (s, 4H) 1.51 (s, 5H) 2.92-3.13 (m, 1H) 3.02 (s, 3H) 4.13-4.19 (m, 2H) 4.81 (s, 1H) 5.04-5.10 (m, 2H) 7.34-7.45 (m, 2H) 7.82-7.90 (m, 2H).
Compound 3a was prepared according to the scheme below.

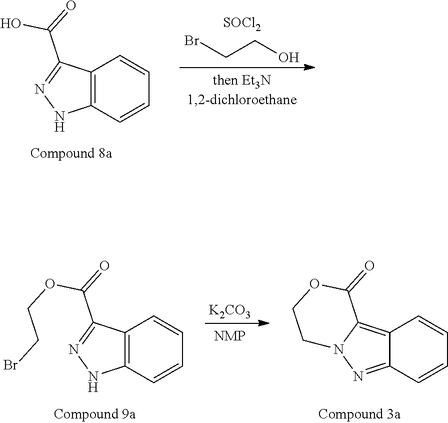

### Preparation of 2-bromoethyl 1H-indazole-3-carboxylate (Compound 9a)

Charged 49.74 kg of 1H-indazole-3-carboxylic acid (Compound 8a), 627.4 kg of 1,2-dichloroethane and 1.12 kg of dimethylformamide (DMF) into 1000 L reactor. Adjusted the temperature of the reactor to 70° C. and added 43.93 kg of thionyl chloride dropwise. Stirred the reaction mixture at 70° C. for 6 hours. Adjusted the temperature to 30° C. and charged 69.00 kg of 2-bromoethanol and then slowly charged 74.49 kg of trimethylamine in the reactor. Agitated the reaction mixture at 25° C. for 22 hours. Charged 249.2 kg of water and agitated the mixture. Separated the phases and removed the aqueous layer. Washed the organic layer with 249.2 kg of water again and removed the aqueous layer. Vacuum distilled the mixture under 50° C. Added the toluene and distilled for change the solvent twice. Adjusted the final amount of the mixture to 298.4 kg. Heated the mixture to 50° C. to generate a solution and then cooled the reaction mixture to 0° C. over 4 hours. Stirred the slurry at 0° C. for 4.5 hour. Filtered the reaction mixture and washed the cake with 99.6 kg of cold toluene. The solids were dried under vacuum under 50° C. to yield 52.79 kg of 2-bromoethyl 1H-indazole-3-carboxylate (Compound 9a).

ESI-MS: m/z 269.0[MH⁺]; ¹H NMR (400 MHz, CDCl₃) δ 3.73 (t, J=6.2 Hz, 2H), 4.80 (t, J=6.2 Hz, 2H), 7.37 (m, 8.0 Hz, 1H), 7.49 (m, 1H), 7.69 (m, 1H), 8.27 (m, 1H), 11.63 (brs, 1H).

### Preparation of 3,4-dihydro-1H-[1,4]oxazino[4,3-b]indazol-1-one (Compound 3a)

Charged 30.0 kg of Compound 9a and 37.50 kg of N-methylpyrrolidone (NMP) into 100 L reactor (reactor A). Charged 18.49 kg of powdered potassium carbonate and 29.95 kg of NMP into 200 L reactor (reactor B). Heated the temperature of the reactor B to 90° C. and added Compound 9a solution into reactor B dropwise. Stirred the reaction mixture at 90° C. for 20 minutes. Cooled the reaction mixture under 80° C. and charged 50.7 kg of toluene and adjusted the temperature under 35° C. Charged 22.96 kg of acetic acid and 225.6 kg of water into 1000 L reactor (reactor C) and adjusted the temperature at 10° C. Reaction mixture was added into the reactor C dropwise and washed with 50.9 kg of toluene at 20° C. Charged 101.2 kg of ethyl acetate and agitated the mixture. Separated the phases and washed the aqueous layer with 101.2 kg of toluene and 101.5 kg of ethyl acetate. Removed the aqueous layer and combined organic layers into reactor C, and then charged 90.6 kg of water and 12.27 kg of sodium chloride. Agitated the mixture and separated the phases and removed the aqueous layer. Washed the organic layer with 90.3 kg of water, and removed the aqueous layer.

Repeated the above operation twice and combined the organic layers through the filter into 500 L reactor (reactor D). Vacuum distilled the mixture under 50° C. Added the butyl acetate and distilled the mixture under 100° C. for change the solvent twice. Adjusted the final amount of the mixture to 84 kg. Heated the mixture over 100° C. to generate a solution and then cooled the reaction mixture to 0° C. over 8 hours. Stirred the slurry at the same temperature for 16.5 hours. Filtered the reaction mixture and washed the cake with 52.5 kg of cold butyl acetate. The solids were dried under vacuum under 50° C. to yield 28.4 kg of 3,4-dihydro-1H-[1,4]oxazino[4,3-b]indazol-1-one (Compound 3a).

ESI-MS: m/z 189.1[MH⁺]; ¹H NMR (400 MHz, CDCl₃) δ 4.76 (m, 2H), 4.83 (m, 2H), 7.39 (m, 1H), 7.39 (m, 1H), 7.85 (d, J=8.4 Hz, 1H), 8.12 (d, J=7.6 Hz, 1H).

### Preparation of 1-(3, 4-dihydro-1H-[1,4]oxazino[4,3-b]indazol-1-yl)-N-methylmethanamine tosylate salt (Compound 1 tosylate salt)

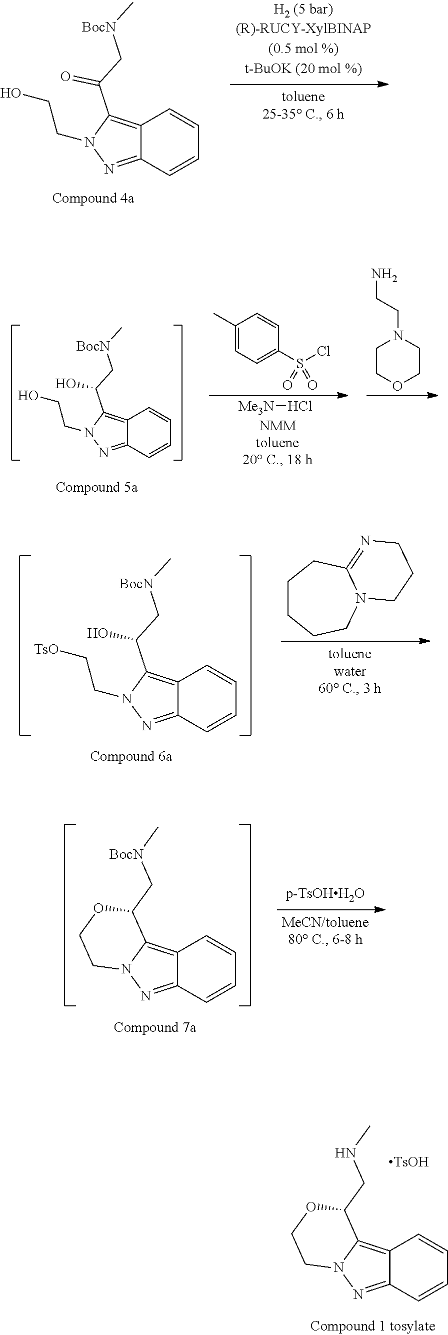

25 g of tert-Butyl (2-(2-(2-hydroxyethyl)-2H-indazol-3-yl)-2-oxoethyl)(methyl)carbamate (Compound 4a), 1.71 g of potassium t-butoxide and 0.46 g of (R)-Rucy-xylBINAP were added to pressure reactor and inerted. 216 g of toluene was added and the mixture was stirred under 5 bar of hydrogen at 30° C. until all the starting material was consumed. The mixture comprising Compound 5a was transferred to a flask and distilled to 165 mL and 0.73 g of trimethylamine hydrochloride and 18.07 g of p-toluenesulfonyl chloride were added. The reaction mixture was cooled to −5° C. and 11.04 g of N-methyl morpholine was added. The reaction was stirred at 20° C. until complete. The reaction was cooled to −5° C., 2.85 g of 4-(2-aminoethyl)morpholine was added and the mixture was stirred for 2 hours. 160.7 g of 8% aqueous KHSO₄ was added and the mixture stirred for 5 min. The stirring was stopped, the aqueous layer was removed and the organic layer was washed with water (2×71 g) to afford the mixture comprising Compound 6a. 17.0 g of 2.1,8-Diazabicyclo[5,4,0]undec-7-ene (DBU) and 36.2 g of de-ionized (DI) water were added to the mixture and reaction was stirred at 60° C. until the reaction was complete. 36.9 g of water was added and the mixture was stirred for 10 min. The aqueous layer was removed and 7.52 g of 1 N HCl and 73.7 g of DI water were added. After mixing for 10 min the aqueous layer was removed and the organic layer was washed with 73.4 g of water. 116.9 g of toluene was added and the mixture was distilled to a final volume of 75 mL (the mixture comprising Compound 7a). 13.70 g of p-toluenesulfonic acid monohydrate and 69.9 g of acetonitrile were added and the mixture was stirred at reflux until all the starting material was consumed. The mixture was slowly cooled to 20° C. and the resulting slurry was filtered and washed with acetonitrile (3×46 g) to yield the desired product. The solid was dried to yield 18.3 g of (R)-1-(3,4-dihydro-1H-[1,4]oxazino[4,3-b]indazol-1-yl)-N-methylmethanamine tosylate salt (Compound 1 tosylate).

¹H NMR (400 MHz, DMSO-d₆) δ ppm 2.27 (s, 3H) 2.67 (t, J=4.9 Hz, 3H) 3.41 (m, 1H) 3.76 (m, 1H) 3.97-4.20 (m, 1H) 4.37-4.51 (m, 3H) 5.57 (dd, J=9.6, 2.5 Hz, 1H) 7.05-7.11 (m, 1H) 7.1 (d, J=7.8 Hz, 2H) 7.28 (ddd, J=8.8, 6.7, 1.0 Hz, 1H) 7.45 (d, J=7.8 Hz, 2H) 7.61 (d, J=8.6 Hz, 1H) 7.78 (d, J=8.5 Hz, 1H) 8.69 (br s, 2H).

### Example 9

### Alternative Preparation of Compound 4a Using a Corning Advanced Flow Reactor (AFR) System

This example focuses on the synthesis of Compound 4a. It includes two reactions: the lithiation of Compound 1a and the ring opening of Compound 3a with lithiated N-Boc-dimethylamine (Compound 2a). Both reactions were carried out under cryogenic conditions. The lithiation reaction is exothermic and the ring opening reaction is instantaneous.

### Overview of the Flow Process

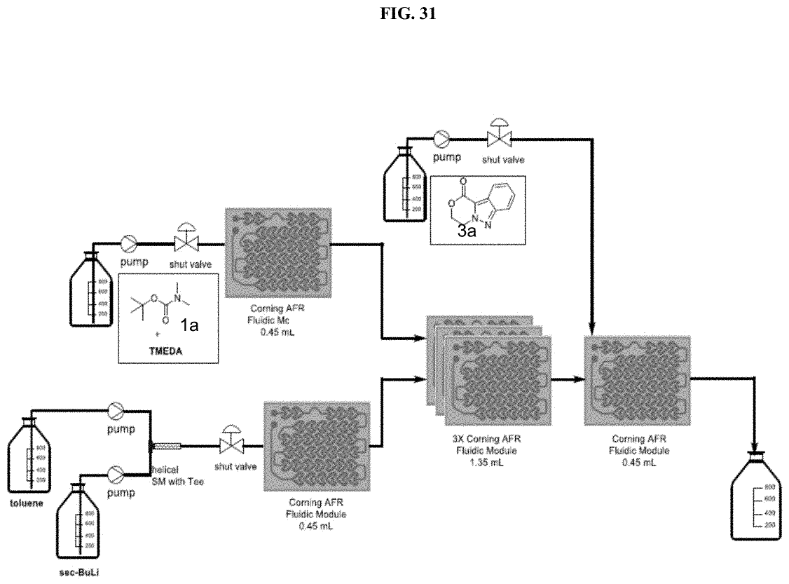

This synthesis was conducted in a Corning AFR Low-Flow reactor system with six total plates: two precooling plates, one mixing plate, two residence time plates, and a final mixing plate before collection. The fluidic configuration of the reactor system is shown in FIG. 31. The system was loaded with three stock solutions, which were prepared as follows:
Stock solution A (Compound 1a solution)
   A tared, dried round bottom flask under nitrogen was charged with Compound 1a (14.5457 g, 97.3% potency, 97.5 mmol, 1.65 eq), TMEDA (22.625g, 3.3 eq), and anhydrous THF (102.7 g). The resulting solution had a light yellowish hue and 399.3 ppm of water by Karl Fisher titrator.
Stock solution B (Compound 3a solution)
   Compound 3a (12.02 g, 92.5% potency, 59.1 mmol, 1 eq) was dissolved in anhydrous THF (166.79 g) under nitrogen. The resulting solution was clear and slightly yellowish with 74.7 ppm of water by Karl Fisher titrator.
Stock solution C (sec-Butyllithium feed solution)
   Using a 3-way valve, a 1.4 M solution of s-BuLi in cyclohexane was withdrawn from the bottle into a plastic syringe before being dispensed through a 1.0 μm glass fiber syringe filter (Pall Acrodisc 25 mm). Filters were replaced as solids accumulated and dispensing by hand became difficult. Three filters were sufficient to filter 100 mL of opaque, light brown s-BuLi solution into a clear, slightly yellow solution under nitrogen.

### Flow Run:

Stock solution C and anhydrous toluene were both pumped at 0.70 mL/min to mix in a 20-element PTFE Tee-mixer for a 1:1 dilution before entering the first plate in the reactor to precool. Similarly, stock solution A was pumped at a rate of 1.41 mL/min into a precooling plate before meeting diluted stock solution C in a mixing plate. The meeting of stock solution A and diluted solution C resulted in a lithiated Compound 1a solution, which flowed through two more residence time plates before mixing with stock solution B, which was pumped at 1.73 mL/min into the final module of the reactor. Despite intermittent clogging, about 37 minutes of total run-time was successful and the efflux solution was collected in three samples summarized below in Table 10.

Reactor temperature was controlled by a chiller with counter-current heat-exchange fluid relative to the process flow. Flow temperatures were monitored at the reactor inlet and outlet by two temperature probes that logged an average of −36° C. and −42° C. over the course of the experiment, respectively.

The conditions for the flow reactions are shown in Tables 8 and 9 below.

TABLE 8
Flow rates for AFR Low-Flow Reactor system

| | Concen- | | | | |
|---|---|---|---|---|---|
| | tration | | Flow Rate | Mass Flow | Flow Rate |
| Material | (M) | Equiv | (mL/min) | (g/min) | (mmol/min) |
| Compound | 0.604 | 1.65 | 1.41 | 1.22 | 0.85 |
| 1a + TMEDA | | | | | |
| (Stock | | | | | |
| solution A) | | | | | |
| s-BuLi (Stock | 1.22 | 1.65 | 0.7 | 0.54 | 0.85 |
| solution C) | | | | | |
| Toluene | ~ | ~ | 0.7 | 0.61 | ~ |
| Compound 3a | 0.298 | 1.00 | 1.73 | 1.58 | 0.52 |
| (B) | | | | | |
| Total | | | 4.55 | 3.94 | |

TABLE 9
Reaction component volume and residence times

| | Mixing | Reactor | Residence |
|---|---|---|---|
| | time | volume | time |
| Reactor component | (s) | (mL) | (s) |
| Sec-BuLi/toluene Precooling HX | N/A | 0.45 | 19 |
| (Stock solution C diluted) | | | |
| Compound 1a stream Precooling HX | N/A | 0.45 | 19 |
| (A) | | | |
| Compound 1a lithiation (A + C) | N/A | 1.35 | 29 |
| Lactone opening [B + (A + C)] | N/A | 0.45 | 6 |

A summary of the results of the run are presented in Table 10 below. An impurity was observed, having the following structure:

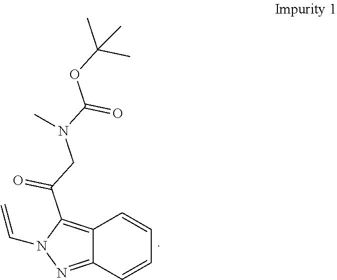 [SMILES: C=Cn1nc2ccccc2c1C(=O)CN(C)C(=O)OC(C)(C)C]

TABLE 10
Summary of samples collected in AFR demonstration run

| | Molar ratio | | |
|---|---|---|---|
| | Compound | | |
| | 4a:Compound | Compound | |
| | 3a ratio | 4a:Impurity | Amount crude solution |
| Sample No. | (conversion) | 1 ratio | collected (g) |
| 1 | >99:1 | 99.4:0.6 | 39.1 |
| 2 | >99:1 | 99.7:0.3 | 96.8 |
| 3 | >99:1 | 99.8:0.2 | 10.5 |
| Average*/Total | >99:1 | 99.6:0.4 | 146.3 |

*Weighted average

### Workup and Isolation:

The three samples described in Table 10 were combined to afford 146.4 g of reaction mixture. The theoretical yield for this amount of reaction mixture was 6.41 g of Compound 4a. The reaction mixture was transferred to a separatory funnel with a rinse of 2×10 mL 2-MeTHF. The organic layer was washed with 1N aqueous HCl (100 mL) (pH ˜6 after separating), followed by 100 mL saturated aqueous NaHCO₃ (pH ˜9 after separating). The organic layer was concentrated under reduced pressure and 120 mL of 2-MeTHF was charged to the residue. The resulting liquors had a water content of 104 ppm by Karl Fischer titration. This mixture was heated to ˜50° C. to afford a cloudy mixture with a small amount of solids present. This was filtered to remove inorganic material. The mixture was concentrated under reduced pressure to a net weight of 40.4 g, when crystallization nucleation was observed.

The resulting mixture was heated to reflux under ambient pressure, affording a homogenous solution. This was cooled to ambient temperature with stirring (stir bar). The liquors contained 2.09% (w/w) Compound 4a, or ca. 10% of the total theoretical yield. The mixture was charged with 15.9 g hexanes and allowed to equilibrate with stirring. After this addition, the liquors contained 0.68% (w/w) Compound 4a. Another addition of 15.8 g hexanes was performed and the mixture was allowed to equilibrate for 10 hours at ambient temperature with stirring before the product was isolated by filtration. Compound 4a (5.27 g) was recovered as a white solid that was determined to be 100.2% potent by the second-generation HPLC assay. This represents an isolated yield of 82%. The liquors (72.0 g) contained 0.29% (w/w) Compound 4a, or 0.21 g., for an additional 3.3% yield. The combined yield was 85.6%.

### Example 10

### Alternative Preparation of Compound 4a Using an Exergy HX Tubular Reactor

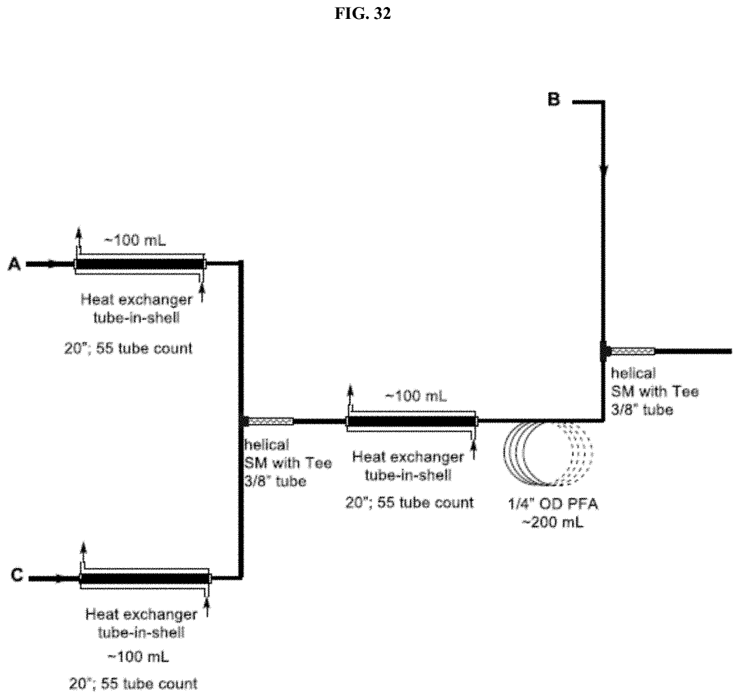

The experiment was conducted in a custom tubular reactor with three exergy heat exchangers: two for precooling stock solutions A′ and C′ (diluted) and one for residence time cooling of lithiated Compound 1a. Each heat exchanger was 5 mL in volume. In order to match residence time, a 2.8 mL loop of ⅛^{th} of an inch OD FEP tubing was coiled tightly around the third heat exchanger before the lithiated Compound 1a solution was mixed with the Compound 3a solution. The fluidic configuration of the reactor system is shown in FIG. 32. The system was loaded with stock solutions, which were prepared as follows:
Stock solution A′ (Compound 1a solution)
   This solution was prepared in slight excess (1.3× scale). Compound 1a (42.5 g, 97.3% potency, 285 mmol) and TMEDA (66.68 g) were dissolved in anhydrous THF (297.10 g). The resulting solution had a light yellowish hue and 419 ppm of water by Karl Fisher titration.
Stock solution B′ (Compound 3a solution)
   Compound 3b (25.10 g, 92.5% potency, 123.4 mmol, 1 eq) was dissolved in anhydrous THF (350.2 g) under nitrogen. The resulting solution was clear and slightly yellowish with 0.8 ppm of water by Karl Fisher titration.
Stock solution C′ (sec-Butyllithium feed solution)
   Using a 3-way valve, s-BuLi was withdrawn from the bottle into a plastic syringe before being dispensed through a 1.0 μm glass fiber syringe filter (Pall Acrodisc 25 mm).

### Experimental Procedure:

During this run, reactor temperatures were not logged. The recirculating chiller temperature was set at −45° C. to achieve the reaction mixture stream operating at around −40° C. Initially, s-BuLi (stock solution C′), toluene, and Compound 1a (stock solution A′) were started at their respective flow rates and allowed to run for two minutes before the Compound 3a (stock solution B′) flow was started. 5 minutes after the start of the first pumps, crude product collection started. Overall, product was collected in period of about 45 min. Through the course of the collection, pressure of the system from upstream (toluene or s-BuLi) pumps were <1.5 barg. The summary of reaction conditions are summarized in Tables 11 and 12.

TABLE 11
Flow rate summary

| | Concen- | | | | |
|---|---|---|---|---|---|
| | tration | | Flow Rate | Mass Flow | Flow Rate |
| Material | (M) | Eq | (mL/min) | (g/min) | (mmol/min) |
| Compound | 0.604 | 1.82 | 6.15 | 5.29 | 3.77 |
| 1a + TMEDA | | | | | |
| (A′) | | | | | |
| s-BuLi (C′) | 1.22 | 1.65 | 2.8 | 2.15 | 3.42 |
| Toluene | ~ | ~ | 2.8 | 2.42 | ~ |
| Compound 3a | 0.298 | 1.00 | 6.94 | 6.32 | 2.07 |
| (B′) | | | | | |
| Total | | | 18.7 | 16.2 | |

TABLE 12
Reaction component volume and residence times

| | Mixing | Reactor | Residence |
|---|---|---|---|
| | time | volume | time |
| Reactor component | (s) | (mL) | (s) |
| Sec-BuLi/toluene Precooling HX | N/A | 5 | 54 |
| Compound 1a stream Precooling HX | N/A | 5 | 49 |
| Compound 1a lithiation | ~0.5 | 8 | 41 |
| Lactone opening | ~0.35 | 2-3 | 8 |

* Estimated based on residence time in static mixer (20 helical elements)

The results of the run are summarized in Table 13 below.

TABLE 13
Conditions and results of modular reactor run

| Circulating Chiller Temperature | | −45° | C. |
|---|---|---|---|
| Total flow rate (throughput) | | 18.7 mL/min (2.07 mmol/ | |
| | | min Compound 3a) | |
| Product collection time | | 45 | min |
| s-BuLi/toluene pump pressure | | 0.9-1.5 barg | |
| Total crude collected with quench | | 768.5 | g |
| Product HPLC | Compound 4a potency | 3.70 | wt % |

| | | |
|---|---|---|
| assay | Compound 4a:Impurity 1 | 98:2 |

### Isolation of Compound 4a

After the reaction was complete, a standard acid (aq. HCl) and base (NaHCO₃) workup was performed, similar to the procedures as described in Example 9. Then, azeotropic distillation with 2-MeTHF and polish filtration were performed to remove inorganic salt, and the concentrated product mass was taken into about 10 volumes CPME and heated to 82° C. until homogeneous. This solution was cooled slowly with agitation to 72° C. at which point about 40 mg of seed crystal was added to induce crystallization. Compound 4a can be crystallized without seed crystals. The suspension was kept at 70° C. for 30 minutes then cooled slowly to 20° C. at a rate of 0.5° C./min. The following morning this suspension was cooled to 0° C. and allowed to stir for 2.5 hours before solids were isolated by vacuum filtration, and the cake was washed with 2×50 mL of cold CPME. Solids were transferred to a tared, amber bottle and dried overnight in vacuum oven at 45° C. to yield 23.8 g of 99.8% pure product. The isolated yield in this case was 77% and losses in the washes and the mother liquor combined were about 3-4%.

An alternative work-up and isolation procedure was performed as follows. 706.5 g of process stream from the flow reactor (after quench and phase separation), containing crude Compound 4a, was washed with 150.3 g of 17% aqueous potassium bicarbonate. After phase separation, the upper layer organic phase was then concentrated to a target volume of 150 mL under vacuum distillation. 434.4 g of toluene and 170.7 g of water were added, and the process stream was heated to approximately 72° C. The aqueous layer was removed, and the organic layer was washed one more times with 171.1 g of water at 72° C. The aqueous layer was removed, and the organic layer was concentrated to a target volume of 300 mL under vacuum distillation. The process stream was then heated to approximately 88° C. to dissolve all the solid contents, and then cooled to 70° C. to facilitate nucleation. The process stream was stirred at the temperature of self-nucleation for approximately 40 minutes and cooled linearly to 10° C. over 2.5 hours. After aging at 10° C. for approximately 1 hour, the slurry was filtered, and the product cake was washed with around 35 g cold toluene (approximately 10° C.) twice. The wet-cake was dried under vacuum at approximately 45° C. to constant weight to yield Compound 4a as a light-yellow solid.

Over a total of 1.5 kg of Compound 4a was produced using these processes. In quantifications tests, the system was run in full scale for about 30 min smoothly and around 10 L reaction mixture was collected. HPLC results indicated the yield was about 95%.

Various preferred embodiments of the invention can be described in the text below:
[1]. A compound:

(R)-1-(3,4-dihydro-1H-[1,4]oxazino[4,3-b]indazol-1-yl)-N-methylmethanamine tosylate (Compound 1 Tosylate);

(R)-1-(3,4-dihydro-1H-[1,4]oxazino[4,3-b]indazol-1-yl)-N-methylmethanamine benzenesulfonate (Compound 1 Besylate);

(R)-1-(3,4-dihydro-1H-[1,4]oxazino[4,3-b]indazol-1-yl)-N-methylmethanamine fumarate (Compound 1 Fumarate);

(R)-1-(3,4-dihydro-1H-[1,4]oxazino[4,3-b]indazol-1-yl)-N-methylmethanamine phosphate (Compound 1 Phosphate); or

(R)-1-(3,4-dihydro-1H-[1,4]oxazino[4,3-b]indazol-1-yl)-N-methylmethanamine hydrochloride (Compound 1 Hydrochloride);

or a hydrate or solvate thereof,

wherein Compound 1 Hydrochloride is crystalline and is Form HB or Form HC.
[2]. A compound of embodiment [1] above, or according to other embodiments of the invention, wherein Compound 1 Tosylate, Compound 1 Besylate, Compound 1 Fumarate, or Compound 1 Phosphate is a solid form.
[3]. A compound of embodiment [1] or [2] above, or according to other embodiments of the invention, wherein the compound is (R)-1-(3,4-dihydro-1H-[1,4]oxazino[4,3-b]indazol-1-yl)-N-methylmethanamine tosylate (Compound 1 Tosylate).
[4]. A compound of embodiment [1], [2], or [3] above, or according to other embodiments of the invention, wherein Compound 1 Tosylate is anhydrous.
[5]. A compound of embodiment [1], [2], [3], or [4] above, or according to other embodiments of the invention, wherein Compound 1 Tosylate is crystalline.
[6]. A compound of embodiment [5] above, or according to other embodiments of the invention, having Form TA.
[7]. A compound of embodiment [6] above, or according to other embodiments of the invention, wherein Form TA has at least one characteristic XRPD peak selected from about 5.7, about 11.3, and about 16.4 degrees 2-theta.
[8]. A compound of embodiment [6] above, or according to other embodiments of the invention, wherein Form TA has at least one characteristic XRPD peak selected from about 5.7, about 11.3, about 16.4, about 16.9, and about 22.6 degrees 2-theta.
[9]. A compound of embodiment [6] above, or according to other embodiments of the invention, wherein Form TA has at least two characteristic XRPD peaks selected from about 5.7, about 11.3, about 16.4, about 16.9, about 18.3, about 22.6, and about 28.4 degrees 2-theta.
[10]. A compound of embodiment [6] above, or according to other embodiments of the invention, wherein Form TA has at least three characteristic XRPD peaks selected from about 5.7, about 11.3, about 16.4, about 16.9, about 18.3, about 22.6, and about 28.4 degrees 2-theta.
[11]. A compound of embodiment [6], [7], [8], [9], or [10] above, or according to other embodiments of the invention, wherein Form TA has an XRPD pattern with characteristic peaks as substantially shown in FIG. 1.
[12]. A compound of embodiment [6], [7], [8], [9], [10], or [11] above, or according to other embodiments of the invention, wherein Form TA has an endotherm peak at a temperature of about 238° C.
[13]. A compound of embodiment [6], [7], [8], [9], [10], or [11] above, or according to other embodiments of the invention, wherein Form TA has a DSC thermogram substantially as depicted in FIG. 2.
[14]. A compound of embodiment [6], [7], [8], [9], [10], [11], [12], or [13] above, or according to other embodiments of the invention, wherein Form TA has a DVS isotherm substantially as depicted in FIG. 4.
[15]. A compound of embodiment [6], [7], [8], [9], [10], [11], [12], [13], or [14] above, or according to other embodiments of the invention, wherein Form TA has a TGA thermogram substantially as depicted in FIG. 3.
[16]. A compound of embodiment [1] or [2] above, or according to other embodiments of the invention, wherein the compound is (R)-1-(3,4-dihydro-1H-[1,4]oxazino[4,3-b]indazol-1-yl)-N-methylmethanamine benzenesulfonate (Compound 1 Besylate).
[17]. A compound of embodiment [1], [2], or [16] above, or according to other embodiments of the invention, wherein Compound 1 Besylate is anhydrous.
[18]. A compound of embodiment [1], [2], [16], or [17] above, or according to other embodiments of the invention, wherein Compound 1 Besylate is crystalline.
[19]. A compound of embodiment [18] above, or according to other embodiments of the invention, having Form BA.
[20]. A compound of embodiment [19] above, or according to other embodiments of the invention, wherein Form BA has at least one characteristic XRPD peak selected from about 5.9, about 11.8, about 17.1, and about 17.3 degrees 2-theta.
[21]. A compound of embodiment [19] above, or according to other embodiments of the invention, wherein Form BA has at least one characteristic XRPD peak selected from about 5.9, about 11.8, about 17.1, about 17.3, about 18.2, about 19.2, and about 19.4 degrees 2-theta.
[22]. A compound of embodiment [19] above, or according to other embodiments of the invention, wherein Form BA has at least two characteristic XRPD peaks selected from about 5.9, about 11.8, about 17.1, about 17.3, about 18.2, about 19.2, about 19.4, about 22.8, about 23.8, about 24.4, and about 25.7 degrees 2-theta.
[23]. A compound of embodiment [19] above, or according to other embodiments of the invention, wherein Form BA has at least three characteristic XRPD peaks selected from about 5.9, about 11.8, about 17.1, about 17.3, about 18.2, about 19.2, about 19.4, about 22.8, about 23.8, about 24.4, and about 25.7 degrees 2-theta.
[24]. A compound of embodiment [19], [20], [21], [22], or [23] above, or according to other embodiments of the invention, wherein Form BA has an XRPD pattern with characteristic peaks as substantially shown in FIG. 5.
[25]. A compound of embodiment [19], [20], [21], [22], [23], or [24] above, or according to other embodiments of the invention, wherein Form BA has an endotherm peak at a temperature of about 225° C.
[26]. A compound of embodiment [19], [20], [21], [22], [23], or [24] above, or according to other embodiments of the invention, wherein Form BA has a DSC thermogram substantially as depicted in FIG. 6.
[27]. A compound of embodiment [19], [20], [21], [22], [23], [24], [25], or [26] above, or according to other embodiments of the invention, wherein Form BA has a DVS isotherm substantially as depicted in FIG. 7.
[28]. A compound of embodiment [1] or [2] above, or according to other embodiments of the invention, wherein the compound is (R)-1-(3,4-dihydro-1H-[1,4]oxazino[4,3-b]indazol-1-yl)-N-methylmethanamine hydrochloride (Compound 1 Hydrochloride).
[29]. A compound of embodiment [1], [2], or [28] above, or according to other embodiments of the invention, wherein Compound 1 Hydrochloride is a hydrate.
[30]. A compound of embodiment [28] above, or according to other embodiments of the invention, having Form HB.
[31]. A compound of embodiment [28] or [29] above, or according to other embodiments of the invention, having Form HC.
[32]. A compound of embodiment [31] above, or according to other embodiments of the invention, wherein Form HC is a mono-hydrate.
[33]. A compound of embodiment [31] or [32] above, or according to other embodiments of the invention, wherein Form HC is a mono-hydrochloride.
[34]. A compound of embodiment [31], [32], or [33] above, or according to other embodiments of the invention, wherein Form HC has at least one characteristic XRPD peak selected from about 8.4, about 10.4, and about 11.0 degrees 2-theta.
[35]. A compound of embodiment [31], [32], or [33] above, or according to other embodiments of the invention, wherein Form HC has at least one characteristic XRPD peak selected from about 8.4, about 10.4, about 11.0, about 14.0, about 16.4, and about 16.9 degrees 2-theta.
[36]. A compound of embodiment [31], [32], or [33] above, or according to other embodiments of the invention, wherein Form HC has at least two characteristic XRPD peaks selected from about 8.4, about 10.4, about 11.0, about 14.0, about 16.4, about 16.9, about 18.3, about 22.1, about 26.4, about 27.1, and about 29.1 degrees 2-theta.
[37]. A compound of embodiment [31], [32], or [33] above, or according to other embodiments of the invention, wherein Form HC has at least three characteristic XRPD peaks selected from about 8.4, about 10.4, about 11.0, about 14.0, about 16.4, about 16.9, about 18.3, about 22.1, about 26.4, about 27.1, and about 29.1 degrees 2-theta.
[38]. A compound of any one of embodiments [31] to [37] above, or according to other embodiments of the invention, wherein Form HC has an XRPD pattern with characteristic peaks as substantially shown in FIG. 12.
[39]. A compound of any one of embodiments [31] to [38] above, or according to other embodiments of the invention, wherein Form HC has endotherm peaks at temperatures of about 131° C. and about 216° C., and an exotherm peak at temperature of about 182° C.
[40]. A compound of any one of embodiments [31] to [38] above, or according to other embodiments of the invention, wherein Form HC has a DSC thermogram substantially as depicted in FIG. 13.
[41]. A compound of any one of embodiments [31] to [40] above, or according to other embodiments of the invention, wherein Form HC has a DVS isotherm substantially as depicted in FIG. 15.
[42]. A compound of any one of embodiments [31] to [41] above, or according to other embodiments of the invention, wherein Form HC has a TGA thermogram substantially as depicted in FIG. 14.
[43]. A compound of embodiment [28] or [29] above, or according to other embodiments of the invention, having Form HD.
[44]. A compound of embodiment [43] above, or according to other embodiments of the invention, wherein Form HD is a di-hydrate.
[45]. A compound of embodiment [43] or [44] above, or according to other embodiments of the invention, wherein Form HD is mono-hydrochloride.
[46]. A compound of embodiment [1] or [2] above, or according to other embodiments of the invention, wherein the compound is (R)-1-(3,4-dihydro-1H-[1,4]oxazino [4,3-b ]indazol -1-yl)-N-methylmethanamine fumarate (Compound 1 Fumarate).
[47]. A compound of embodiment [1], [2], or [46] above, or according to other embodiments of the invention, wherein Compound 1 Fumarate is crystalline.
[48]. A compound of embodiment [1], [2], [46], or [47] above, or according to other embodiments of the invention, wherein Compound 1 Fumarate is a hydrate.
[49]. A compound of embodiment [1], [2], [46], or [47] above, or according to other embodiments of the invention, wherein Compound 1 Fumarate is anhydrous.
[50]. A compound of embodiment [46], [47], or [48] above, or according to other embodiments of the invention, having Form FA.
[51]. A compound of embodiment [50] above, or according to other embodiments of the invention, wherein Form FA is a di-hydrate.
[52]. A compound of embodiment [50] or [51] above, or according to other embodiments of the invention, wherein Form FA has at least one characteristic XRPD peak selected from about 9.9, about 11.4, and about 12.0 degrees 2-theta.
[53]. A compound of embodiment [50] or [51] above, or according to other embodiments of the invention, wherein Form FA has at least one characteristic XRPD peak selected from about 9.9, about 11.4, about 12.0, about 14.8, about 15.5, about 15.8, about 16.3, and about 17.2 degrees 2-theta.
[54]. A compound of embodiment [50] or [51] above, or according to other embodiments of the invention, wherein Form FA has at least two characteristic XRPD peaks selected from about 9.9, about 11.4, about 12.0, about 14.8, about 15.5, about 15.8, about 16.3, about 17.2, about 18.7, about 19.3, about 19.8, about 21.7, about 24.0, about 25.7, and about 27.7 degrees 2-theta.
[55]. A compound of embodiment [50] or [51] above, or according to other embodiments of the invention, wherein Form FA has at least three characteristic XRPD peaks selected from about 9.9, about 11.4, about 12.0, about 14.8, about 15.5, about 15.8, about 16.3, about 17.2, about 18.7, about 19.3, about 19.8, about 21.7, about 24.0, about 25.7, and about 27.7 degrees 2-theta.
[56]. A compound of any one of embodiments [50] to [55] above, or according to other embodiments of the invention, wherein Form FA has an XRPD pattern with characteristic peaks as substantially shown in FIG. 20.
[57]. A compound of any one of embodiments [50] to [56] above, or according to other embodiments of the invention, wherein Form FA has endotherm peaks at temperatures of about 101° C. and about 152° C.
[58]. A compound of any one of embodiments [50] to [56] above, or according to other embodiments of the invention, wherein Form FA has a DSC thermogram substantially as depicted in FIG. 21.
[59]. A compound of any one of embodiments [50] to [58] above, or according to other embodiments of the invention, wherein Form FA has a DVS isotherm substantially as depicted in FIG. 23.
[60]. A compound of any one of embodiments [50] to [59] above, or according to other embodiments of the invention, wherein Form FA has a TGA thermogram substantially as depicted in FIG. 22.
[61]. A compound of embodiment [46], [47], or [48] above, or according to other embodiments of the invention, having Form FB.
[62]. A compound of embodiment [61] above, or according to other embodiments of the invention, wherein Form FB is a mono-hydrate.
[63]. A compound of embodiment [61] or [62] above, or according to other embodiments of the invention, wherein Form FB has at least one characteristic XRPD peak selected from about 8.8, about 9.1, about 14.6, and about 14.8 degrees 2-theta.
[64]. A compound of embodiment [61] or [62] above, or according to other embodiments of the invention, wherein Form FB has at least one characteristic XRPD peak selected from about 8.8, about 9.1, about 11.9, about 12.4, about 14.6, about 14.8, about 16.0, about 16.6, and about 17.7 degrees 2-theta.
[65]. A compound of embodiment [61] or [62] above, or according to other embodiments of the invention, wherein Form FB has at least two characteristic XRPD peaks selected from about 8.8, about 9.1, about 11.9, about 12.4, about 14.6, about 14.8, about 16.0, about 16.6, about 17.7, about 19.4, about 20.3, about 20.8, about 21.7, about 23.7, about 23.9, and about 24.6 degrees 2-theta.
[66]. A compound of embodiment [61] or [62] above, or according to other embodiments of the invention, wherein Form FB has at least three characteristic XRPD peaks selected from about 8.8, about 9.1, about 11.9, about 12.4, about 14.6, about 14.8, about 16.0, about 16.6, about 17.7, about 19.4, about 20.3, about 20.8, about 21.7, about 23.7, about 23.9, and about 24.6 degrees 2-theta.
[67]. A compound of any one of embodiments [61] to [66] above, or according to other embodiments of the invention, wherein Form FB has an XRPD pattern with characteristic peaks as substantially shown in FIG. 24.
[68]. A compound of any one of embodiments [61] to [67] above, or according to other embodiments of the invention, wherein Form FB has an endotherm peak at a temperature of about 159° C.
[69]. A compound of any one of embodiments [61] to [67] above, or according to other embodiments of the invention, wherein Form FB has a DSC thermogram substantially as depicted in FIG. 25.
[70]. A compound of any one of embodiments [61] to [69] above, or according to other embodiments of the invention, wherein Form FB has a DVS isotherm substantially as depicted in FIG. 27.
[71]. A compound of any one of embodiments [61] to [70] above, or according to other embodiments of the invention, wherein Form FB has a TGA thermogram substantially as depicted in FIG. 26.
[72]. A compound of embodiment [1] or [2] above, or according to other embodiments of the invention, wherein the compound is (R)-1-(3,4-dihydro-1H-[1,4]oxazino[4,3-b ]indazol-1-yl)-N-methylmethanamine phosphate (Compound 1 Phosphate).
[73]. A compound of embodiment [1], [2], or [72] above, or according to other embodiments of the invention, wherein Compound 1 Phosphate is crystalline.
[74]. A compound of embodiment [72] or [73] above, or according to other embodiments of the invention, having Form PA.
[75]. A compound of embodiment [74] above, or according to other embodiments of the invention, wherein Form PA has at least one characteristic XRPD peak selected from about 4.7, about 6.6, about 7.1, and about 8.4 degrees 2-theta.
[76]. A compound of embodiment [74] above, or according to other embodiments of the invention, wherein Form PA has at least one characteristic XRPD peak selected from about 4.7, about 6.6, about 7.1, about 8.4, about 9.0, and about 9.4 degrees 2-theta.
[77]. A compound of embodiment [74] above, or according to other embodiments of the invention, wherein Form PA has at least one characteristic XRPD peak selected from about 4.7, about 6.6, about 7.1, about 8.4, about 9.0, about 9.4, about 10.0, about 10.3, about 10.6, and about 11.1 degrees 2-theta.
[78]. A compound of embodiment [74] above, or according to other embodiments of the invention, wherein Form PA has at least two characteristic XRPD peaks selected from about 4.7, about 6.6, about 7.1, about 8.4, about 9.0, about 9.4, about 10.0, about 10.3, about 10.6, about 11.1, about 13.2, about 13.6, about 14.2, about 14.5, about 14.6, about 15.4, and about 16.1 degrees 2-theta.
[79]. A compound of embodiment [74] above, or according to other embodiments of the invention, wherein Form PA has at least three characteristic XRPD peaks selected from about 4.7, about 6.6, about 7.1, about 8.4, about 9.0, about 9.4, about 10.0, about 10.3, about 10.6, about 11.1, about 13.2, about 13.6, about 14.2, about 14.5, about 14.6, about 15.4, about 16.1, about 18.3, about 18.8, about 20.3, about 23.6, and about 25.1 degrees 2-theta.
[80]. A compound of any one of embodiments [74] to [79] above, or according to other embodiments of the invention, wherein Form PA has an XRPD pattern with characteristic peaks as substantially shown in FIG. 28.
[81]. A compound of any one of embodiments [74] to [80] above, or according to other embodiments of the invention, wherein Form PA has endotherm peaks at temperatures of about 88° C. and about 191° C.
[82]. A compound of any one of embodiments [74] to [80] above, or according to other embodiments of the invention, wherein Form PA has a DSC thermogram substantially as depicted in FIG. 29.
[83]. A compound of any one of embodiments [74] to [82] above, or according to other embodiments of the invention, wherein Form PA has a TGA thermogram substantially as depicted in FIG. 30.
[84]. A pharmaceutical composition comprising one or more pharmaceutically acceptable excipients and a compound of any one of embodiments [1] to [83] above, or according to other embodiments of the invention.
[85]. A method for treating a neurological or psychiatric disorder in a patient, comprising administering to said patient an effective amount of a compound of any one of embodiments [1] to [83] above, or according to other embodiments of the invention.
[86]. A method of embodiment [85] above, or according to other embodiments of the invention, wherein the neurological or psychiatric disorder is depression, bipolar disorder, pain, schizophrenia, obsessive compulsive disorder, addiction, social disorder, attention deficit hyperactivity disorder, an anxiety disorder, autism, cognitive impairments, or suicidality.
[87]. A method of embodiment [85] above, or according to other embodiments of the invention, wherein the neurological or psychiatric disorder is suicidality.
[88]. A method of embodiment [87] above, or according to other embodiments of the invention, wherein the suicidality is acute suicidality, suicidal thoughts, or suicidal behavior.
[89]. A method of embodiment [85] above, or according to other embodiments of the invention, wherein the neurological or psychiatric disorder is pain.
[90]. A method of embodiment [85] above, or according to other embodiments of the invention, wherein the neurological or psychiatric disorder is depression.
[91]. A method of embodiment [90] above, or according to other embodiments of the invention, wherein the depression is treatment-resistant depression (TRD), major depressive disorder (MDD), unipolar depression, bipolar depression or depression associated with another disease or disorder.
[92]. A method of embodiment [90] above, or according to other embodiments of the invention, wherein the depression is treatment-resistant depression.
[93]. A method of embodiment [90] above, or according to other embodiments of the invention, wherein the depression is MDD.
[94]. A method of embodiment [90] above, or according to other embodiments of the invention, wherein the depression is depression associated with another disease/disorder.
[95]. A process for preparing crystalline (R)-1-(3,4-dihydro-1H-[1,4]oxazino[4,3-b]indazol-1-yl)-N-methylmethanamine benzenesulfonate (Compound 1 Besylate) comprising precipitating Compound 1 Besylate from a solution comprising Compound 1, benzensulfonic acid, and S2, wherein S2 is a solvent.
[96]. A process for preparing crystalline (R)-1-(3,4-dihydro-1H-[1,4]oxazino[4,3-b]indazol-1-yl)-N-methylmethanamine fumarate (Compound 1 Fumarate) comprising precipitating Compound 1 Fumarate from a solution comprising Compound 1, fumaric acid, and S3, wherein S3 is a solvent.
[97]. A process for preparing crystalline (R)-1-(3,4-dihydro-1H-[1,4]oxazino[4,3-b]indazol-1-y1)-N-methylmethanamine hydrochloride (Compound 1 Hydrochloride) comprising precipitating Compound 1 Hydrochloride from a solution comprising Compound 1, hydrochloric acid, and S4, wherein S4 is a solvent.
[98]. A process for preparing crystalline (R)-1-(3,4-dihydro-1H-[1,4]oxazino[4,3-b]indazol-1-yl)-N-methylmethanamine phosphate (Compound 1 Phosphate) comprising precipitating Compound 1 Phosphate from a solution comprising Compound 1, phosphoric acid, and S5, wherein S5 is a solvent.
[99]. A process for preparing crystalline (R)-1-(3,4-dihydro-1H-[1,4]oxazino[4,3-b]indazol-1-yl)-N-methylmethanamine tosylate (Compound 1 Tosylate) comprising precipitating Compound 1 Tosylate from a solution comprising Compound 1, toluenesulfonic acid, and S1, wherein S1 is a solvent.
[100]. A process of embodiment [99] above, or according to other embodiments of the invention, wherein S1 comprises chloroform, methanol, acetone, water, toluene, acetonitrile, or a mixture thereof.
[101]. A process of preparing (R)-1-(3,4-dihydro-1H-[1,4]oxazino[4,3-b]indazol-1-yl)-N-methylmethanamine tosylate (Compound 1 Tosylate) comprising reacting tert-butyl (R)-((3,4-dihydro-1H-[1,4]oxazino[4,3-b]indazol-1-yl)methyl)(methyl)carbamate (Compound 7a) with p-toluenesulfonic acid.
[102]. A process of embodiment [101] above, or according to other embodiments of the invention, wherein the p-toluenesulfonic acid is p-toluenesulfonic acid mono-hydrate.
[103]. A process of embodiment [101] or [102] above, or according to other embodiments of the invention, wherein the reacting of Compound 7a with p-toluenesulfonic acid is carried out in the presence of S1a, wherein S1a is a solvent.
[104]. A process of embodiment [103] above, or according to other embodiments of the invention, wherein S1a is a polar aprotic solvent, non-polar solvent, or a mixture thereof.
[105]. A process of embodiment [104] above, or according to other embodiments of the invention, wherein the polar aprotic solvent of S1a is acetonitrile.
[106]. A process of embodiment [104] above, or according to other embodiments of the invention, wherein the non-polar solvent of S1a is toluene.
[107]. A process of any one of embodiments [101] to [106] above, or according to other embodiments of the invention, wherein the reacting of Compound 7a with p-toluenesulfonic acid is carried out at a temperature of about 50° C. to about 100° C.
[108]. A process of any one of embodiments [101] to [107] above, or according to other embodiments of the invention, wherein between about 1 and about 5 molar equivalents of p-toluenesulfonic acid are used per molar equivalent of Compound 7a.
[109]. A process of any one of embodiments [101] to [108] above, or according to other embodiments of the invention, wherein Compound 7a is prepared by a process comprising reacting (R)-2-(3-(2-((tert-butoxycarbonyl)(methyl)amino)-1-hydroxyethyl)-2H-indazol-2-yl)ethyl 4-methylbenzenesulfonate (Compound 6a) with 2,3,4,6,7,8,9,10-octahydropyrimido[1,2-a]azepine.
[110]. A process of preparing tert-butyl (R)-((3,4-dihydro-1H-[1,4]oxazino[4,3-b]indazol-1-yl)methyl)(methyl)carbamate (Compound 7a) comprising reacting (R)-2-(3-(2-((tert-butoxycarbonyl)(methyl)amino)-1-hydroxyethyl)-2H-indazol-2-yl)ethyl 4-methylbenzenesulfonate (Compound 6a) with 2,3,4,6,7,8,9,10-octahydropyrimido[1,2-a]azepine.
[111]. A process of embodiment [109] or [110] above, or according to other embodiments of the invention, wherein the reacting of Compound 6a with 2,3,4,6,7,8,9,10-octahydropyrimido[1,2-a]azepine is carried out in the presence of S2a, wherein S2a is a solvent.
[112]. A process of embodiment [111] above, or according to other embodiments of the invention, wherein S2a is a protic solvent, non-polar solvent, or a mixture thereof.
[113]. A process of embodiment [112] above, or according to other embodiments of the invention, wherein the protic solvent of S2a is water.
[114]. A process of embodiment [112] above, or according to other embodiments of the invention, wherein the non-polar solvent of S2a is toluene.
[115]. A process of any one of embodiments [109] to [114] above, or according to other embodiments of the invention, wherein the reacting of Compound 6a with 2,3,4,6,7,8,9,10-octahydropyrimido[1,2-a]azepine is carried out at a temperature of about 30° C. to about 90° C.
[116]. A process of any one of embodiments [109] to [115] above, or according to other embodiments of the invention, wherein between about 1 and about 5 molar equivalents of 2,3,4,6,7,8,9,10-octahydropyrimido[1,2-a]azepine are used per molar equivalent of Compound 6a.
[117]. A process of any one of embodiments [109] to [116] above, or according to other embodiments of the invention, wherein Compound 6a is prepared by a process comprising reacting tert-butyl (R)-(2-hydroxy-2-(2-(2-hydroxyethyl)-2H-indazol-3-yl)ethyl)(methyl)carbamate (Compound 5a) with p-toluenesulfonyl chloride.
[118]. A process of preparing (R)-2-(3-(2-((tert-butoxycarbonyl)(methyl)amino)-1-hydroxyethyl)-2H-indazol-2-yl)ethyl 4-methylbenzenesulfonate (Compound 6a) comprising reacting tert-butyl (R)-(2-hydroxy-2-(2-(2-hydroxyethyl)-2H-indazol-3-yl)ethyl)(methyl)carbamate (Compound 5a) with p-toluenesulfonyl chloride.
[119]. A process of embodiment [117] or [118] above, or according to other embodiments of the invention, wherein the reacting of Compound 5a with p-toluenesulfonyl chloride is carried out in the presence of trimethylamine hydrochloride and N-methylmorpholine.
[120]. A process of embodiment [119] above, or according to other embodiments of the invention, wherein the reacting of Compound 5a with p-toluenesulfonyl chloride is further carried out in the presence of 2-morpholinoethan-1-amine.
[121]. A process of any one of embodiments [117] to [120] above, or according to other embodiments of the invention, wherein the reacting of Compound 5a with p-toluenesulfonyl chloride is carried out in the presence of S3a, wherein S3a is a solvent.
[122]. A process of embodiment [121] above, or according to other embodiments of the invention, wherein S3a is a non-polar solvent.
[123]. A process of embodiment [122] above, or according to other embodiments of the invention, wherein the non-polar solvent of S3a is toluene.
[124]. A process of any one of embodiments [117] to [123] above, or according to other embodiments of the invention, wherein the reacting of Compound 5a with p-toluenesulfonyl chloride is carried out at a temperature of about −10° C. to about 40° C.
[125]. A process of any one of embodiments [117] to [124] above, or according to other embodiments of the invention, wherein between about 1 and about 5 molar equivalents of p-toluenesulfonyl chloride are used per molar equivalent of Compound 5a.
[126]. A process of any one of embodiments [119] to [125] above, or according to other embodiments of the invention, wherein between about 0.02 and about 0.2 molar equivalents of trimethylamine hydrochloride are used per molar equivalent of Compound 5a.
[127]. A process of any one of embodiments [119] to [126] above, or according to other embodiments of the invention, wherein between about 1 and about 5 molar equivalents of N-methylmorpholine are used per molar equivalents of Compound 5a.
[128]. A process of any one of embodiments [120] to [127] above, or according to other embodiments of the invention, wherein between about 0.05 and about 0.5 molar equivalents of 2-morpholinoethan-1-amine are used per molar equivalent of Compound 5a.
[129]. A process of any one of embodiments [117] to [128] above, or according to other embodiments of the invention, wherein Compound 5a is prepared by a process comprising reacting tert-butyl (2-(2-(2-hydroxyethyl)-2H-indazol-3-yl)-2-oxoethyl)(methyl)carbamate (Compound 4a) with a reducing agent.
[130]. A process of preparing tert-butyl (R)-(2-hydroxy-2-(2-(2-hydroxyethyl)-2H-indazol-3-yl)(methyl)carbamate (Compound 5a) comprising reacting tert-butyl (2-(2-(2-hydroxyethyl)-2H-indazol-3-yl)-2-oxoethyl)(methyl)carbamate (Compound 4a) with a reducing agent.
[131]. A process of embodiment [129] or [130] above, or according to other embodiments of the invention, wherein the reducing agent is hydrogen in the presence of a transition metal catalyst.
[132]. A process of embodiment [131] above, or according to other embodiments of the invention, wherein the transition metal catalyst is a ruthenium catalyst.
[133]. A process of embodiment [132] above, or according to other embodiments of the invention, wherein the ruthenium catalyst is (R)-RUCY-XylBINAP.
[134]. A process of any one of embodiments [129] to [133] above, or according to other embodiments of the invention, wherein the reacting of Compound 4a with a reducing agent is carried out in the presence of B1a, wherein B1a is an alkali metal alkoxide base.
[135]. A process of embodiment [134] above, or according to other embodiments of the invention, wherein B1a is a potassium alkoxide base.
[136]. A process of embodiment [134] or [135] above, or according to other embodiments of the invention, wherein B1a is potassium tert-butoxide.
[137]. A process of any one of embodiments [129] to [136] above, or according to other embodiments of the invention, wherein the reacting of Compound 4a with a reducing agent is carried out in the presence of S4a, wherein S4a is a solvent.
[138]. A process of embodiment [137] above, or according to other embodiments of the invention, wherein the S4a is a non-polar solvent
[139]. A process of embodiment [138] above, or according to other embodiments of the invention, wherein the non-polar solvent of S4a is toluene.
[140]. A process of any one of embodiments [129] to [139] above, or according to other embodiments of the invention, wherein the reacting of Compound 4a with a reducing agent is carried out at a temperature of about 0° C. to about 60° C.
[141]. A process of any one of embodiments [131] to [140] above, or according to other embodiments of the invention, wherein the pressure of hydrogen is between about 1 bar and about 10 bar.
[142]. A process of any one of embodiments [131] to [141] above, or according to other embodiments of the invention, wherein between about 0.0001 and about 0.05 molar equivalents of transition metal catalyst are used per molar equivalent of Compound 4a.
[143]. A process of any one of embodiments [134] to [142] above, or according to other embodiments of the invention, wherein between about 0.05 and about 0.5 molar equivalents of alkali metal alkoxide base are used per molar equivalent of Compound 4a.
[144]. A process of any one of embodiments [129] to [143] above, or according to other embodiments of the invention, wherein Compound 4a is prepared by a process comprising reacting 3,4-dihydro-1H-[1,4]oxazino[4,3-b]indazol-1-one (Compound 3a) with lithiated tert-butoxycarbonyldimethylamine (Compound 2a).
[145]. A process of preparing tert-butyl (2-(2-(2-hydroxyethyl)-2H-indazol-3-yl)-2-oxoethyl)(methyl)carbamate (Compound 4a) comprising reacting 3,4-dihydro-1H-[1,4]oxazino[4,3-b]indazol-1-one (Compound 3a) with lithiated tert-butoxycarbonyldimethylamine (Compound 2a).
[146]. A process of embodiment [144] or [145] above, or according to other embodiments of the invention, wherein the reacting of Compound 3a with Compound 2a is carried out in the presence of S5a, wherein S5a is a solvent.
[147]. A process of embodiment [146] above, or according to other embodiments of the invention, wherein S5a is a polar aprotic solvent.
[148]. A process of embodiment [147] above, or according to other embodiments of the invention, wherein the polar aprotic solvent of S5a is tetrahydrofuran.
[149]. A process of any one of embodiments [144] to [148] above, or according to other embodiments of the invention, wherein the reacting of Compound 3a with lithiated tert-butoxycarbonyldimethylamine is carried out at a temperature of about −120° C. to about −40° C.
[150]. A process of any one of embodiments [144] to [149] above, or according to other embodiments of the invention, wherein between about 1 and about 5 molar equivalents of Compound 2a are used per molar equivalent of Compound 3a.
[151]. A process of any one of embodiments [144] to [150] above, or according to other embodiments of the invention, wherein lithiated tert-butoxycarbonyldimethylamine (Compound 2a) is prepared by a process comprising reacting tert-butoxycarbonyldimethylamine (Compound 1a) with a lithiating agent.
[152]. A process of preparing lithiated tert-butoxycarbonyldimethylamine (Compound 2a) comprising reacting tert-butoxycarbonyldimethylamine (Compound 1a) with a lithiating agent.
[153]. A process of embodiment [151] or [152] above, or according to other embodiments of the invention, wherein the lithiating agent is an alkyllithium reagent.
[154]. A process of embodiment [153] above, or according to other embodiments of the invention, wherein the lithiating agent is sec-butyllithium.
[155]. A process of any one of embodiments [151] to [154] above, or according to other embodiments of the invention, wherein the reacting of Compound 1a with a lithiating agent is carried out in the presence of a ligand and S6a, wherein S6a is a solvent.
[156]. A process of embodiment [155] above, or according to other embodiments of the invention, wherein the ligand is an amine.
[157]. A process of embodiment [156] above, or according to other embodiments of the invention, wherein ligand is tetramethylethylenediamine.
[158]. A process of any one of embodiments [151] to [157] above, or according to other embodiments of the invention, wherein S6a is a polar aprotic solvent.
[159]. A process of embodiment [158] above, or according to other embodiments of the invention, wherein the polar aprotic solvent of S6a is tetrahydrofuran.
[160]. A process of any one of embodiments [151] to [159] above, or according to other embodiments of the invention, wherein the reacting of Compound 1a with a lithiating agent is carried out at a temperature of about −120° C. to about 10° C.
[161]. A process of any one of embodiments [151] to [160] above, or according to other embodiments of the invention, wherein between about 0.5 and about 5 molar equivalents of lithiating agent are used per molar equivalent of Compound 1a.
[162]. A process of any one of embodiments [155] to [161] above, or according to other embodiments of the invention, wherein between about 0.5 and about 3 molar equivalents of the ligand are used per molar equivalent of Compound 1a.
[163]. A process of any one of embodiments [144] to [150] above, or according to other embodiments of the invention, wherein Compound 3a is prepared by a process comprising reacting 2-bromoethyl 1H-indazole-3-carboxylate (Compound 9a) with B2a, wherein B2a is an alkali metal base.
[164]. A process of preparing 3,4-dihydro-1H-[1,4]oxazino[4,3-b]indazol-1-one (Compound 3a) comprising reacting 2-bromoethyl 1H-indazole-3-carboxylate (Compound 9a) with B2a, wherein B2a is an alkali metal base.
[165]. A process of embodiment [163] or [164] above, or according to other embodiments of the invention, wherein B2a is an alkali metal carbonate.
[166]. A process of embodiment [165] above, or according to other embodiments of the invention, wherein B2a is potassium carbonate.
[167]. A process of any one of embodiments [163] to [166] above, or according to other embodiments of the invention, wherein the reacting of Compound 9a with B2a is carried out in the presence of S7a, wherein S7a is a solvent.
[168]. A process of embodiment [167] above, or according to other embodiments of the invention, wherein S7a is a polar aprotic solvent.
[169]. A process of embodiment [168] above, or according to other embodiments of the invention, wherein the polar aprotic solvent of S7a is N-methylpyrrolidone.
[170]. A process of any one of embodiments [163] to [169] above, or according to other embodiments of the invention, wherein the reacting of Compound 9a with B2a is carried out at a temperature of about 50° C. to about 120° C.
[171]. A process of any one of embodiments [163] to [170] above, or according to other embodiments of the invention, wherein between about 0.5 and about 2 molar equivalents of B2a are used per molar equivalent of Compound 9a.
[172]. A process of any one of embodiments [163] to [171] above, or according to other embodiments of the invention, wherein Compound 9a is prepared by a process comprising reacting 1H-indazole-3-carboxylic acid (Compound 8a) with 2-bromoethanol, a chlorinating agent, and an amine compound.
[173]. A process of preparing 2-bromoethyl 1H-indazole-3-carboxylate (Compound 9a) comprising reacting 1H-indazole-3-carboxylic acid (Compound 8a) with 2-bromoethanol, a chlorinating agent, and an amine compound.
[174]. A process of embodiment [172] or [173] above, or according to other embodiments of the invention, wherein the chlorinating agent is thionyl chloride.
[175]. A process of any one of embodiments [172] to [174] above, or according to other embodiments of the invention, wherein the amine is a tertiary amine.
[176]. A process of embodiment [175] above, or according to other embodiments of the invention, wherein the tertiary amine is trimethylamine.
[177]. A process of any one of embodiments [172] to [176] above, or according to other embodiments of the invention, wherein the reacting of Compound 8a with 2-bromoethanol, a chlorinating agent, and an amine compound, is carried out in the presence of S8a, wherein S8a is a solvent.
[178]. A process of embodiment [177] above, or according to other embodiments of the invention, wherein S8a is a polar aprotic solvent.
[179]. A process of embodiment [178] above, or according to other embodiments of the invention, wherein the polar aprotic solvent of S8a is dimethylformamide, 1,2-dichloroethane, or a mixture thereof.
[180]. A process of any one of embodiments [172] to [179] above, or according to other embodiments of the invention, wherein the reacting of Compound 8a with a chlorinating agent is carried out at a temperature of about 50° C. to about 100° C. to form a first mixture.
[181]. A process of embodiment [180] above, or according to other embodiments of the invention, wherein 2-bromoethanol and the amine are added to the first mixture at a temperature of about 20° C. to about 40° C.
[182]. A process of any one of embodiments [172] to [181] above, or according to other embodiments of the invention, wherein between about 1 and about 2 molar equivalents of chlorinating agent are used per molar equivalent of Compound 8a.
[183]. A process of any one of embodiments [172] to [182] above, or according to other embodiments of the invention, wherein between about 1 and about 2 molar equivalents of 2-bromoethanol are used per molar equivalent of Compound 8a.
[184]. A process of any one of embodiments [172] to [183] above, or according to other embodiments of the invention, wherein between about 1 and about 4 molar equivalents of amine are used per molar equivalent of Compound 8a.
[185]. A process of preparing Compound 1 Tosylate comprising:

reacting Compound 1a with a lithiating agent to produce Compound 2a;

reacting Compound 2a with Compound 3a to produce Compound 4a;

reacting Compound 4a with a reducing agent to produce Compound 5a;

reacting Compound 5a with p-toluenesulfonyl chloride to produce Compound 6a;

reacting Compound 6a with a base to produce Compound 7a; and

reacting Compound 7a with p-toluenesulfonic acid to produce Compound 1 Tosylate.
[186]. A process of preparing Compound 1 Tosylate comprising:

reacting Compound 1a with a lithiating agent to produce Compound 2a;

reacting Compound 8a with 2-bromoethane to produce Compound 9a;

reacting Compound 9a with an alkali metal base to produce Compound 3a;

reacting Compound 2a with Compound 3a to produce Compound 4a;

reacting Compound 4a with a reducing agent to produce Compound 5a;

reacting Compound 5a with p-toluenesulfonyl chloride to produce Compound 6a;

reacting Compound 6a with a base to produce Compound 7a; and

reacting Compound 7a with p-toluenesulfonic acid to produce Compound 1 Tosylate.
[187]. A compound which is tert-butyl (R)-((3,4-dihydro-1H-[1,4]oxazino[4,3-b]indazol-1-yl)methyl)(methyl)carbamate (Compound 7a) or a salt thereof.
[188]. A compound which is tert-butyl (2-(2-(2-hydroxyethyl)-2H-indazol-3-yl)-2-oxoethyl)(methyl)carbamate (Compound 4a) or a salt thereof.
[189]. A compound which is 3,4-dihydro-1H-[1,4]oxazino[4,3-b]indazol-1-one (Compound 3a) or a salt thereof.
[190]. A compound which is 1H-indazole-3-carboxylic acid (Compound 8a) or a salt thereof.
[191]. A compound which is 2-bromoethyl 1H-indazole-3-carboxylate (Compound 9a) or a salt thereof.
[192]. Compound 1, or a pharmaceutically acceptable salt, hydrate or solvate thereof, prepared by a process of any one of [95] to [186] above, or according to other embodiments of the invention.
[193]. A process of any one of embodiments [144] to [162] above, or according to other embodiments of the invention, wherein the reacting of Compound 3a with Compound 2a is performed in a continuous flow process.
[194]. A process of embodiment [193] above, or according to other embodiments of the invention, wherein the continuous flow process comprises:

adding Compound 1a to a first reactor and contacting said Compound 1a with a lithiating agent to provide Compound 2a; and

transferring said Compound 2a to a second reactor and contacting Compound 2a with Compound 3a to provide Compound 4a.
[195]. A process of embodiment [194] above, or according to other embodiments of the invention, wherein the continuous flow process is carried out at an average temperature between about −80° C. and about −20° C.
[196]. A process of embodiment [194] above, or according to other embodiments of the invention, wherein the continuous flow process has a total residence time between about 0.2 minutes and about 5 minutes.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound
(R)-1-(3,4-dihydro-1H-[1,4]oxazino[4,3-b]indazol-1-yl)-N-methylmethanamine tosylate (Compound 1 Tosylate);
(R)-1-(3,4-dihydro-1H-[1,4]oxazino[4,3-b]indazol-1-yl)-N-methylmethanamine benzenesulfonate (Compound 1 Besylate);
(R)-1-(3,4-dihydro-1H-[1,4]oxazino[4,3-b]indazol-1-yl)-N-methylmethanamine fumarate (Compound 1 Fumarate);
(R)-1-(3,4-dihydro-1H-[1,4]oxazino[4,3-b]indazol-1-yl)-N-methylmethanamine phosphate (Compound 1 Phosphate); or
(R)-1-(3,4-dihydro-1H-[1,4]oxazino[4,3-b]indazol-1-yl)-N-methylmethanamine hydrochloride (Compound 1 Hydrochloride);
or a hydrate or solvate thereof,
wherein Compound 1 Hydrochloride is crystalline and is Form HB or Form HC.

2. The compound of claim 1, wherein Compound 1 Tosylate, Compound 1 Besylate, Compound 1 Fumarate, or Compound 1 Phosphate is a solid form.

3-5. (canceled)

6. The compound of claim 1 having Form TA.

7-15. (canceled)

16. The compound of claim 1, wherein the compound is (R)-1-(3,4-dihydro-1H-[1,4] oxazino[4,3-b]indazol-1-yl)-N-methylmethanamine benzenesulfonate (Compound 1 Besylate).

17-18. (canceled)

19. The compound of claim 1 having Form BA.

20-27. (canceled)

28. The compound of claim 1, wherein the compound is (R)-1-(3,4-dihydro-1H-[1,4]oxazino[4,3-b]indazol-1-yl)-N-methylmethanamine hydrochloride (Compound 1 Hydrochloride).

29. (canceled)

30. The compound of claim 28 having Form HB.

31. The compound of claim 28 having Form HC.

32-42. (canceled)

43. The compound of claim 28 or 29 having Form HD.

44-45. (canceled)

46. The compound of claim 1, wherein the compound is (R)-1-(3,4-dihydro-1H-[1,4] oxazino[4,3-b]indazol-1-yl)-N-methylmethanamine fumarate (Compound 1 Fumarate).

47-49. (canceled)

50. The compound of claim 46 having Form FA.

51-60. (canceled)

61. The compound of claim 46 having Form FB.

62-71. (canceled)

72. The compound of claim 1, wherein the compound is (R)-1-(3,4-dihydro-1H-[1,4]oxazino[4,3-b]indazol-1-yl)-N-methylmethanamine phosphate (Compound 1 Phosphate).

73. (canceled)

74. The compound of claim 72 having Form PA.

75-83. (canceled)

84. A pharmaceutical composition comprising a compound of claim 1, and one or more pharmaceutically acceptable excipients.

85. A method for treating a neurological or psychiatric disorder in a patient, comprising administering to said patient an effective amount of a compound of any one claim 1.

86. The method according to claim 85, wherein the neurological or psychiatric disorder is depression, bipolar disorder, pain, schizophrenia, obsessive compulsive disorder, addiction, social disorder, attention deficit hyperactivity disorder, an anxiety disorder, autism, cognitive impairments, or suicidality.

87-89. (canceled)

90. The method according to claim 85, wherein the neurological or psychiatric disorder is depression.

91. The method according to claim 90, wherein the depression is treatment-resistant depression (TRD), major depressive disorder (MDD), unipolar depression, bipolar depression or depression associated with another disease or disorder.

92-94. (canceled)

95. A process for preparing:
a) crystalline (R)-1-(3,4-dihydro-1H-[1,4]oxazino[4,3-b]indazol-1-yl)-N-methylmethanamine benzenesulfonate (Compound 1 Besylate) comprising precipitating Compound 1 Besylate from a solution comprising Compound 1, benzensulfonic acid, and S2, wherein S2 is a solvent; or
b) crystalline (R)-1-(3,4-dihydro-1H-[1,4]oxazino[4,3-b]indazol-1-yl)-N-methylmethanamine fumarate (Compound 1 Fumarate) comprising precipitating Compound 1 Fumarate from a solution comprising Compound 1, fumaric acid, and S3, wherein S3 is a solvent; or
c) crystalline (R)-1-(3,4-dihydro-1H-[1,4]oxazino[4,3-b]indazol-1-yl)-N-methylmethanamine hydrochloride (Compound 1 Hydrochloride) comprising precipitating Compound 1 Hydrochloride from a solution comprising Compound 1, hydrochloric acid, and S4, wherein S4 is a solvent; or
d) crystalline (R)-1-(3,4-dihydro-1H-[1,4]oxazino[4,3-b]indazol-1-yl)-N-methylmethanamine phosphate (Compound 1 Phosphate) comprising precipitating Compound 1 Phosphate from a solution comprising Compound 1, phosphoric acid, and S5, wherein S5 is a solvent; or
e) crystalline (R)-1-(3,4-dihydro-1H-[1,4]oxazino[4,3-b]indazol-1-yl)-N-methylmethanamine tosylate (Compound 1 Tosylate) comprising precipitating Compound 1 Tosylate from a solution comprising Compound 1, toluenesulfonic acid, and S1, wherein S1 is a solvent; or
f) (R)-1-(3,4-dihydro-1H-[1,4]oxazino[4,3-b]indazol-1-yl)-N-methylmethanamine tosylate (Compound 1 Tosylate) comprising reacting tert-butyl (R)-((3,4-dihydro-1H-[1,4]oxazino[4,3-b]indazol-1-yl)methyl)(methyl)carbamate (Compound 7a) with p-toluenesulfonic acid; or
g) tert-butyl (R)-((3,4-dihydro-1H-[1,4]oxazino[4,3-b]indazol-1-yl)methyl)(methyl)carbamate (Compound 7a) comprising reacting (R) -2-(3-(2-((tert-butoxycarbonyl)(methyl)amino)-1-hydroxyethyl)-2H-indazol-2-yl)ethyl 4-methylbenzenesulfonate (Compound 6a) with 2,3,4,6,7,8,9,10-octahydropyrimido[1,2-a]azepine; or
h) (R)-2-(3-(2-((tert-butoxycarbonyl)(methyl)amino)-1-hydroxyethyl)-2H-indazol-2-yl)ethyl 4-methylbenzenesulfonate (Compound 6a) comprising reacting tert-butyl (R)-(2-hydroxy-2-(2-(2-hydroxyethyl)-2H-indazol-3-yl)ethyl)(methyl)carbamate (Compound 5a) with p-toluenesulfonyl chloride; or
i) tert-butyl (R)-(2-hydroxy-2-(2-(2-hydroxyethyl)-2H-indazol-3-yl)ethyl)(methyl)carbamate (Compound 5a) comprising reacting tert-butyl (2-(2-(2-hydroxyethyl)-2H-indazol-3-yl)-2-oxoethyl)(methyl)carbamate (Compound 4a) with a reducing agent; or
j) tert-butyl (2-(2-(2-hydroxyethyl)-2H-indazol-3-yl)-2-oxoethyl)(methyl)carbamate (Compound 4a) comprising reacting 3,4-dihydro-1H-[1,4]oxazino[4,3-b]indazol-1-one (Compound 3a) with lithiated tert-butoxycarbonyldimethylamine (Compound 2a); or
k) lithiated tert-butoxycarbonyldimethylamine (Compound 2a) comprising reacting tert-butoxycarbonyldimethylamine (Compound 1a) with a lithiating agent;
l) 3,4-dihydro-1H-[1,4]oxazino[4,3-b]indazol-1-one (Compound 3a) comprising reacting 2-bromoethyl 1H-indazole-3-carboxylate (Compound 9a) with B2a, wherein B2a is an alkali metal base; or
m) 2-bromoethyl 1H-indazole-3-carboxylate (Compound 9a) comprising reacting 1H-indazole-3-carboxylic acid (Compound 8a) with 2-bromoethanol, a chlorinating agent, and an amine compound.

96-184. (canceled)

185. A process of preparing Compound 1 Tosylate comprising:
A) reacting Compound 1a with a lithiating agent to produce Compound 2a;
reacting Compound 2a with Compound 3a to produce Compound 4a;
reacting Compound 4a with a reducing agent to produce Compound 5a;
reacting Compound 5a with p-toluenesulfonyl chloride to produce Compound 6a;
reacting Compound 6a with a base to produce Compound 7a; and
reacting Compound 7a with p-toluenesulfonic acid to produce Compound 1 Tosylate: or
B) reacting Compound 1a with a lithiating agent to produce Compound 2a;
reacting Compound 8a with 2-bromoethane to produce Compound 9a;
reacting Compound 9a with an alkali metal base to produce Compound 3a;
reacting Compound 2a with Compound 3a to produce Compound 4a;
reacting Compound 4a with a reducing agent to produce Compound 5a;
reacting Compound 5a with p-toluenesulfonyl chloride to produce Compound 6a;
reacting Compound 6a with a base to produce Compound 7a, and
reacting Compound 7a with p-toluenesulfonic acid to produce Compound 1 Tosylate.

186. (canceled)

187. A compound which is selected from:
a) tert-butyl (R)-((3,4-dihydro-1H-[1,4]oxazino[4,3-b]indazol-1-yl)methyl)(methyl)carbamate (Compound 7a) or a salt thereof;
b) tert-butyl (2-(2-(2-hydroxyethyl)-2H-indazol-3-yl)-2-oxoethyl)(methyl)carbamate (Compound 4a) or a salt thereof;
c) 3,4-dihydro-1H-[1,4]oxazino[4,3-b]indazol-1-one (Compound 3a) or a salt thereof;
d) 1H-indazole-3-carboxylic acid (Compound 8a) or a salt thereof; and
e) 2-bromoethyl 1H-indazole-3-carboxylate (Compound 9a) or a salt thereof.

188-196. (canceled)
